SEQUENCE LISTING (36 sequences)

SEQ ID NO: 1 - DNA, 23 bp, synthetic construct
  source 1..23: mol_type = other DNA
ccagccaggacagaaatgcctcg

SEQ ID NO: 2 - DNA, 26 bp, synthetic construct
  source 1..26: mol_type = other DNA
atgtgagcaaaaggccagcaaaaggc

SEQ ID NO: 3 - DNA, 22 bp, synthetic construct
  source 1..22: mol_type = other DNA
ccggaattgccagctggggcgc

SEQ ID NO: 4 - DNA, 27 bp, synthetic construct
  source 1..27: mol_type = other DNA
cttacttggtgccgaatatcttgtcgc

SEQ ID NO: 5 - DNA, 46 bp, synthetic construct
  source 1..46: mol_type = other DNA
gccttttgctggccttttgctcacatcactcttgcgcgaggaaagc

SEQ ID NO: 6 - DNA, 50 bp, synthetic construct
  source 1..50: mol_type = other DNA
gcgacaagatattcggcaccaagtaaggggaaggcattaaccgatacttg

SEQ ID NO: 7 - DNA, 42 bp, synthetic construct
  source 1..42: mol_type = other DNA
gcgccccagctggcaattccggccgactggatacgcaacacc

SEQ ID NO: 8 - DNA, 45 bp, synthetic construct
  source 1..45: mol_type = other DNA
cgaggcatttctgtcctggctggcctgttcggactctcgatgttc

SEQ ID NO: 9 - DNA, 45 bp, synthetic construct
  source 1..45: mol_type = other DNA
gccttttgctggccttttgctcacatcagaacctcatcgccatgc

SEQ ID NO: 10 - DNA, 49 bp, synthetic construct
  source 1..49: mol_type = other DNA
gcgacaagatattcggcaccaagtaagtcctctccttgttgatgccctg

SEQ ID NO: 11 - DNA, 42 bp, synthetic construct
  source 1..42: mol_type = other DNA
gcgccccagctggcaattccgggggaatgtcgtctcacgcag

SEQ ID NO: 12 - DNA, 42 bp, synthetic construct
  source 1..42: mol_type = other DNA
cgaggcatttctgtcctggctgggtttccggcaaggtcaagg

SEQ ID NO: 13 - DNA, 46 bp, synthetic construct
  source 1..46: mol_type = other DNA
gccttttgctggccttttgctcacattgggcgtacagttcggtatc

SEQ ID NO: 14 - DNA, 50 bp, synthetic construct
  source 1..50: mol_type = other DNA
gcgacaagatattcggcaccaagtaaggtgacaaagcaacgcatcttgtg

SEQ ID NO: 15 - DNA, 43 bp, synthetic construct
  source 1..43: mol_type = other DNA
gcgccccagctggcaattccggtctgccgaagaaagatgcctg

SEQ ID NO: 16 - DNA, 43 bp, synthetic construct
  source 1..43: mol_type = other DNA
cgaggcatttctgtcctggctggagaccgtccatctcgtctgc

SEQ ID NO: 17 - DNA, 23 bp, synthetic construct
  source 1..23: mol_type = other DNA
gggaaggcattaaccgatacttg

SEQ ID NO: 18 - DNA, 43 bp, synthetic construct
  source 1..43: mol_type = other DNA
caagtatcggttaatgccttcccccgactggatacgcaacacc

SEQ ID NO: 19 - DNA, 22 bp, synthetic construct
  source 1..22: mol_type = other DNA
tcctctccttgttgatgccctg

SEQ ID NO: 20 - DNA, 42 bp, synthetic construct
  source 1..42: mol_type = other DNA
cagggcatcaacaaggagaggagggaatgtcgtctcacgcag

SEQ ID NO: 21 - DNA, 23 bp, synthetic construct
  source 1..23: mol_type = other DNA
gtgacaaagcaacgcatcttgtg

SEQ ID NO: 22 - DNA, 44 bp, synthetic construct
  source 1..44: mol_type = other DNA
cacaagatgcgttgctttgtcactctgccgaagaaagatgcctg

SEQ ID NO: 23 - DNA, 21 bp, synthetic construct
  source 1..21: mol_type = other DNA
caaggatctgatggcgcaggg

SEQ ID NO: 24 - DNA, 40 bp, synthetic construct
  source 1..40: mol_type = other DNA
atggtacctcctgggactgcattgcagggcttcccaacct

SEQ ID NO: 25 - DNA, 58 bp, synthetic construct
  source 1..58: mol_type = other DNA
gatcgtgatcccctgcgccatcagatccttgtcaggcgaaaggacgcaggcgcaacaa

SEQ ID NO: 26 - DNA, 44 bp, synthetic construct
  source 1..44: mol_type = other DNA
gcagtcccaggaggtaccatatgcgaatgcctcgcagaacgttc

SEQ ID NO: 27 - DNA, 57 bp, synthetic construct
  source 1..57: mol_type = other DNA
tcatttttcgaactgcgggtggctccaagcgctggccttgcgcaggttgaccatgaa

SEQ ID NO: 28 - DNA, 51 bp, synthetic construct
  source 1..51: mol_type = other DNA
tggagccacccgcagttcgaaaaatgatggcgcgccatcagaagacttgat

SEQ ID NO: 29 - protein, 1009 aa, Desulfovibrio vulgaris
  source 1..1009: note = strain Hildenborough
MTVTRRHFLKLSAGAAVAGAFTGLGLSLAPTVARAELQKLQWAKQTTSICCYCAVGCGLIVHTAKDGQGRAVNVEGDPDHPINEGSLCPKGASIFQLGENDQRGTQPLYRAPFSDTWKPVTWDFALTEIAKRIKKTRDASFTEKNAAGDLVNRTEAIASFGSAAMDNEECWAYGNILRSLGLVYIEHQARIUHSPTVPALAESFGRGAMTNHWNDLANSDCILIMGSNAAENHPIAFKWVLRAKDKGATLIHVDPRFTRTSARCDVYAPIRSGADIPFLGGLIKYILDNKLYFTDYVREYTNASLIVGEKFSFKDGLFSGYDAANKKYDKSMWAFELDANGVPKRDPALKHPRCVINLLKKHYERYNLDKVAAITGTSKEQLQQVYKAYAATGKPDKAGTIMYAMGWTQHSVGVQNIRAMAMIQLLLGNIGVAGGGVNALRGESNVQGSTDQGLLAHIWPGYNPVPNSKAATLELYNAATPQSKDPMSVNWWQNRPKYVASYLKALYPDEEPAAAYDYLPRIDAGRKLTDYFWLNIFEKMDKGEFKGLFAWGMNPACGGANANKNRKAMGKLEWLVNVNLFENETSSFWKGPGMNPAEIGTEVFFLPCCVSIEKEGSVANSGRWMQWRYRGPKPYAETKPDGDIMLDMFKKVRELYAKEGGAYPAPIAKLNIADWEEHNEFSPTKVAKLMNGYFLKDTEVGGKQFKKGQQVPSFAFLTADGSTCSGNWLHAGSFTDAGNLMARRDKTQTPEQARIGLFPNWSFCWPVNRRILYNRASVDKTGKPWNPAKAVIEWKDGKWVGDVVDGGGDPGTKHPFIMQTHGFGALYGPGREEGPFPEHYEPLECPVSKNPFSKQLHNPVAFQIEGEKKAVCDPRYPFIGTTYRVTEHWQTGLMTRRCAWLVEAEPQIFCEISKELAKLRGIGNGDTVKVSSLRGALEAVAIVTERIRPFKIEGVDVHMVGLPWHYGWMVPKNGGDTANLLTPSAGDPNTGIPETKAFMVDVRKVWSHP

SEQ ID NO: 30 - protein, 236 aa, Desulfovibrio vulgaris
  source 1..236: note = strain Hildenborough
MGKMFFVDLSRCTACRGCQIACKQWKNLPAEETRNTGSHQNPPDLSYVTLKTVRFTEKSRKGPGIDWLFFPEQCRHCVEPPCKGQADVDLEGAVVKDETTGAVLFTELTAKVDGESVRSACPYDIPRIDPVTKRLSKCDMCNDRVQNGLLPACVKTCPTGTMNFGDEQEMLALAEKRLAEVKKTYPGAVLGDPNDVRVVYLFTRDPKDFYEHAVADLAPSMMTRQQLFARLFRPRA

SEQ ID NO: 31 - protein, 1003 aa, Desulfovibrio vulgaris
  source 1..1003: note = strain Hildenborough
MRMPRRTFIKLASASAGALAFAGLGQSLAPTVARAAELKIAKAKVTPSVCCFCAVGCGLLVYTDTQTGRSINIEGDPDHPTNEGTLCPKGASIWQTTERSKRVTKVLYRAPGAAAWEEKSWDWALPRIARKIKETRDATFELTNDKGQTVNRTRGIASVGSAAVDNEEGWLMQAMMRALGLVYIESHARIUHSSTVGALAESYGRGAMTNHWIDLKNSDVILIMGSNAAENHPISFKWVTRAQERGATLIHVDPRFTRTSAKADIHAHIRSGTDIAFFGGLIRYIMENDLFFRQYVVDYTNASYIVGADYGFADGLFTGFDAEKGTYNTKKWAFAADENGMTLKDPTLNDPRCVFQLMKAHYARYDRKTVSDVTGISEEQLATLWSTFASTGKPDRAGTILYAMGQCQHTVGVQNIRALSMIQLLLGNIGIAGGGVNALRGESNVQGTTDISLLCDNLSGYLPTPKASWATFDDYVKGTTPVDKDPKSANWWSNRGKYLASYMKSVYPTASHQDGYLWHPKVDDGKITDYSWLQIFERMSKGGFKGAFVWGQNPCAGGANAGKNRKAMETLDWMVVVNLFENESSLFWKGPGVDPAKVKTEVFFLPACMSVEKGGSIANSGRWLQWREPGPKPMGDSRSDGDIVLDLYDEIRKLYREEKGAFPEPVLALDTDYRTDGKYDHHKVAKTLNGKFLTDVTIGDKTYKAGQQVPGFAMLQADGSTTSGCWIFTGCYTDAGNMMARRDRTQTPEQAAIGLFPNWSYAWPANRRILYNRAAVDMTGKPFDPKRAVIAWNGEKWVGDVPDGGWKPGEKLPFIMIREGRGQLFGPGRVDGPFPEHYEPFESPLESHPFSKQRVNPTALAFSHEPKAVRDKRYPFICTTYRVTEQWQSGTMTRNTGWLKEMQPEGFCEISRELAKELGIANGDAVVLESLRGKVQVVAIVTPRLKPFKVMGEVMHEVGIPWQFGWGQHVGKGDSANLLSPSVGDPNTGIPETKVFMVNLRKA

SEQ ID NO: 32 - protein, 260 aa, Desulfovibrio vulgaris
  source 1..260: note = strain Hildenborough
MRPLGSLPQGMRRVSRRHIREAYMNGKTFFIDQTRCTACRGCQAACKQWKKFGSIETRNTGSYQNPPDLGPSTFKLVRFNEVEVDGKLKWLFFPEQCRHCLEPPCKMVADALIPGAIVQDAETGAVLYTEKTKGLDYMEVRSACPYDIPRQDPATGLLTKCNMCIDRVQNGMLPACVKTCPTGTMHFGDRADMLELAQKRLGEVKKRSPAALLADSDLVRVLYLCELAPANYHSHMVAEADTSRIGPFSRRALLRLRPFA

SEQ ID NO: 33 - protein, 430 aa, Desulfovibrio vulgaris
  source 1..430: note = strain Hildenborough
MDYPVWQLWGINGGLLVAIISVLHVFVAQFAVGGGFLLVHAATRAERAGSAEATFWVKRFTRFFLLLSMVFGGVSGVGIWVIISLISPAATSLLVHRFVYAWATEWVFFLGEIVALLVYYYGFGKMRFEDHRNVGLLYGLFAYLSLFMINGIITFMLTPGAWMQTQGFWDGFFNPTFWPSLLFRSALCAMLSGLFGLVWASLIDDERARELMTGVTRLWALVPIPFMAVGAWWYLGALPAPQLEMVLRRTADIRPFLEAFPWVLAALMVGGLAFFTRLPSGARVPVAALLLVLGLCQVGTFEWVRETGRRPWIVHGVIWSNGITPEAGAKSDLEGMLKVARWSGVKQVAEANMHEAGRELWQVQCANCHGLGAPMIDILPRIANRPLTGVEALITGQGALQGYMPPFVGTPAERRALATYLVNERDARRH

SEQ ID NO: 34 - protein, 354 aa, Desulfovibrio vulgaris
  source 1..354: note = strain Hildenborough
MEPASLIPLADAMPGPAWWFEILLLLTFVLHLLLMNTVLGGTVIVAVHALRKPASEQGPVRDFATRLPTALALAINLGVPPLLFVQVLYGNFFYTSSVIMALWWLGIVGLVMLAYYGLYIFAINHDELGTRRIAVIVLALLLLVATSFVFSNNMTLMLQPERWLAWFERRDGALLNLGDPTLIPRWLHMLVGSIAIGGLALSLFHVRRASWGAPDAAEGIETGLRWFSHATYAQIALGIWFLMALPRDLMLRFMGGDTLATAAFILGLIGTALALLAARARSVALAVGAAVGTVTVMAVMRASLREGYLAPWAQPQPVNFVETSPLVLFLASAVCGVAAIIWMLRLMRSSRKGA

SEQ ID NO: 35 - protein, 581 aa, Desulfovibrio vulgaris
  source 1..581: note = strain Hildenborough
MPSKQSPRLTAFYYFAWLVAVVALLGIFAAGGLERPARTTGETPAAKAPAASAAAPSPYMPVTSIPPFDADRADYILLAWSTQGMQMVTDAEPRWTMLPPEAALRAQLVRRGPGPAPVTEGVRITWRVEREHATLEGAPVSGAAGEMKLEQGGSTFAADGIGIMPYRASGGFNAYPTVTVEAHDDEGRLLARTAAVLPVSTEVGCRNCHGGAWKRDGLAGISPATADDVLTVHDRINGTDLVAQAAKAPVECRSCHSGAPEAPAPSLSAALHGWHANYMTDGGAETCASCHPSSPQGATRFMRGLHKAKALDCTRCHGAMEDHALSLLRWEEKAGRKSAARLARELKPRAVSAVDDINPRAPWAMEPDCTGCHDFAQKPATATASAFNKWTASEKDRFSARADDMGNLRCPTCHGAAHAEYPAENPFGRDLDNMPPIQYQQHAKVMGGGNNCSVCHREPLPMEASAHHPVVIRKTIPITLPAGTVLTKPAVLFPHDTHGSMDCGTCHHKGYQEGKQTCATKGCHDRPRPDTIEGLGEVGYFRNAFHGKERGCMTCHLALREKGKAAGPVGCRDCHTPGNNQ

SEQ ID NO: 36 - DNA, 8054 bp, Desulfovibrio vulgaris
  source 1..8054: mol_type = genomic DNA; strain = Hildenborough
atggaacccgcctctctcatcccgttggctgacgccatgcccggcccggcgtggtggttcgaaatcctgcttctgctgaccttcgtcctgcatctgctgctcatgaacacggttcttggcggcacggtcatcgtggcggtgcacgcgttgcgcaaacctgcgtccgaacaggggccggtgcgcgacttcgccacccgcctgcccactgcactggcgcttgccatcaacctcggcgtgccgccactgctcttcgtgcaggtgctctatggcaacttcttctacacgagttccgtcatcatggcgctctggtggctcggcatcgtcggtctggtgatgctcgcctactacggcctgtacatcttcgccatcaatcacgacgaactgggcacgcggcgcatcgccgtcatcgttctggcgctcctgcttctggtggccacgtcgttcgtcttcagcaacaacatgacgctcatgctccagccggaacgctggctggcatggttcgagcggcgtgacggcgcgcttttgaacctcggcgacccgacactgataccgcgctggctgcacatgctcgtgggatccatcgccatcggcggtctggcgctttcgctcttccatgtgcgacgcgcctcgtggggggcacctgacgcggcggagggcatcgagacggggctgcgctggttcagccatgccacgtatgcgcagatagccctgggcatctggttcctcatggcattgccccgcgacctgatgctgcgcttcatggggggggacactctcgcgactgcggccttcatcctcggtctgatcggaacggcccttgccctgctggcggcccgcgcgcgtagcgtggccctcgccgtgggggcagccgtcggcacggtcaccgtcatggcggtgatgcgcgcctcattgcgtgaggggtatcttgccccatgggcgcaaccccaacccgtgaacttcgtcgaaacctcaccgctggtgctttttctcgcgtctgcggtctgcggtgtcgccgccatcatctggatgctccgcctcatgcgcagcagtcgaaagggggcctagcacatggactatcccgtatggcaactctgggggatcaacggtggcctgcttgtggccatcatctccgttctgcatgtgttcgtcgcccagttcgcagtggggggcggtttcctgctcgtccatgccgccacgcgcgcagaacgcgcggggtcggcggaagccacgttctgggtgaaacggttcacccgtttcttcctgctgctcagcatggtgttcggcggtgtatccggcgtgggcatctgggtcatcatctcgctcatcagtccggcagccacgagcctgctggtgcatcgtttcgtctatgcctgggccaccgaatgggtgttcttcctcggcgagatagtggcgttactcgtctactactacggtttcgggaagatgcgcttcgaagaccaccgcaatgtggggctcttgtacggcctgttcgcgtatctctcgctgttcatgatcaacggcatcatcaccttcatgctcacacccggtgcgtggatgcagacgcagggcttctgggacggcttcttcaatcccaccttctggccctcgctgctcttccgcagtgccctgtgcgccatgctctcggggctgttcggccttgtgtgggcctcgctcatcgacgacgagcgcgcacgggaactcatgacgggcgtgacgcggctgtgggcactcgtccccattcccttcatggccgttggcgcatggtggtacctcggggcgctgcctgcaccgcaactcgagatggtgctgcggcgaaccgccgacatccggcccttcctcgaagccttcccgtgggtactggctgccctcatggtgggcgggctggccttcttcacgcggcttccctccggggcgcgcgtgcctgtggctgcgctgctgctggtgctcggcctgtgtcaggtcggaaccttcgagtgggtgcgcgagacgggcagacgcccgtggattgtccacggtgtcatctggtccaacggcatcacgcccgaagccggtgccaagtccgacctcgaagggatgctgaaggtcgcgcggtggtctggcgtcaagcaggttgccgaagcgaacatgcatgaggcaggacgcgaactgtggcaggtgcagtgcgccaactgccacggcctcggagcccccatgatcgacatcctgccccgcatcgcgaacaggccactcaccggggtcgaggcgttgatcaccgggcagggagcgttacagggctacatgccgccgttcgtgggaacccccgcagaacggagggccctcgccacctacctcgtcaacgagcgcgacgcacggcgtcattagcccccgcatgaaagaggagtagattcatgccatccaagcagtcgccaagactcaccgccttctactatttcgcatggcttgtcgccgtcgtcgccctgctcggcatcttcgccgcgggcgggctggagcgcccggcacggacgacgggcgagacgcctgcggccaaggccccagcggcatcggcagcggccccgtctccgtatatgcccgtcacgtcgataccgcccttcgatgcggacagggccgactacatactgctggcgtggagtacgcagggtatgcagatggtgacggacgccgaaccccggtggacgatgctgccccccgaggcggcattgcgggcgcaactcgtgcgccgcgggcccggccccgcgccggtgacagagggcgtacgcatcacgtggcgtgtcgaacgcgaacacgccacccttgaaggtgcacccgtctccggtgcagcaggcgagatgaaactggagcaggggggcagcacgttcgccgccgacggcatcggcatcatgccctatcgcgcttccggcgggttcaatgcctacccgacggtcaccgttgaggcccacgacgacgaaggcaggcttcttgcccgtaccgccgccgtgttgcccgtctccacggaggtggggtgtcgcaactgtcatggcggcgcctggaagcgcgacgggctggccggaatctccccggcgacggccgatgacgtgctcaccgtccatgaccgcatcaacggtaccgacctcgtcgcgcaggctgccaaggcccctgtggagtgccgttcgtgccacagcggggcacccgaggccccggcccccagcctttcggcggccctgcacggctggcatgccaactacatgaccgacggcggcgccgagacatgtgcaagctgccatccttcttcgccgcaaggcgccacgcgcttcatgcgcgggctgcacaaggccaaggcactggactgcacgcgttgccatggtgccatggaagaccacgccctctccctgctgcgctgggaggagaaggctggacgcaagagtgcggcacgtctcgcaagggaactcaagccccgcgccgtgtccgcggtcgatgacatcaacccgcgtgcgccgtgggccatggaacccgactgcaccggctgccatgatttcgcccagaagcctgccacggccacggcatcggcattcaacaagtggacggcttcggagaaggaccgcttctcggcccgcgccgacgacatgggcaacctgcgttgtccgacatgccacggtgcggcacacgccgaatatcccgccgagaatcccttcggccgcgacctcgataacatgccgcccatccagtaccagcagcacgccaaggtcatgggcggcggcaacaactgcagcgtctgccaccgtgagccattgcccatggaggcttcggcgcaccatcccgtggtcatccgcaagaccattcccatcaccctgcccgcaggtacggtactcaccaagcccgccgtgctcttccctcatgacacccacggttcgatggattgcggaacctgtcaccacaagggctatcaggagggcaagcagacctgcgccaccaagggctgccatgacaggccccgtcccgacaccatcgagggtctgggcgaagtgggctacttccgcaacgccttccatggcaaggaacgcgggtgcatgacatgccaccttgcgctgcgtgaaaagggcaaggccgccggtcccgtcggctgtcgcgactgtcacactccgggcaacaatcagtagagaggggagacacatcatgcgaatgcctcgcagaacgttcatcaagctcgcttcggcttccgcaggcgctctcgccttcgccgggctggggcagagcctcgcgcccacggtggcgcgggctgcggaactcaagatcgccaaggcgaaggtcacaccctctgtctgctgcttctgtgcggtgggatgcgggttgctggtctacaccgacacccagaccggacgctccatcaacatcgagggcgaccccgaccatcccaccaacgaagggacgctgtgccccaagggtgcgtccatctggcagaccaccgaacgcagcaagcgtgtcaccaaggtgctgtaccgtgcacccggtgccgcggcgtgggaggagaagtcgtgggactgggccctgccgcgcatcgcccgcaagatcaaggagacgcgcgacgccaccttcgaactcaccaatgacaaggggcagaccgtcaaccgcacccggggcatagcctcggtgggctcggccgccgtcgacaacgaggagggctggctcatgcaagccatgatgcgcgcgctcggcctcgtgtacatcgagagtcacgcacgtatctgacacagctcgactgtgggggctctggcagagtcctacggacgcggtgcgatgacgaatcactggatcgacctcaagaacagcgacgtcattctcatcatggggagtaacgccgccgagaaccatcccatatccttcaagtgggtgacgcgcgcgcaggaacgcggtgcaacgctcatccatgtggacccgcgcttcacgcgcacatccgccaaggccgacatccacgcccacatcaggtcgggcacggacatcgccttcttcggcggtctcatccggtacatcatggagaacgacctgttcttcaggcagtacgtggtcgactacaccaacgcctcgtatatcgtcggtgcggactacggtttcgccgatggtcttttcaccggcttcgacgccgagaagggaacctacaacaccaagaaatgggccttcgccgccgacgagaacggcatgaccctgaaagaccccacgctgaacgacccgcgctgcgtctttcaactcatgaaggcccactacgcccgctatgacaggaagaccgtctccgacgtgacgggcatctccgaagagcagctggcgacgctgtggagtaccttcgcctcgacgggcaagcccgacagggcgggaaccatcctctatgccatggggcagtgccagcacaccgtgggcgtacagaacatccgtgcgctttcgatgatacagctgctgctgggcaacatcggcatcgcagggggcggggtcaacgcgctgcgcggcgaatccaacgtgcagggcaccaccgacatctcgctgctgtgcgacaacctctccggctacctgcccacccccaaggcctcgtgggccaccttcgacgactatgtgaagggcacgacgccggtggacaaggacccgaagagcgccaactggtggtcgaaccgcggcaagtatctcgcctcgtacatgaagtcggtgtatcccacggccagccatcaggacggctacctctggcaccccaaggtcgatgacgggaagataaccgactactcgtggttgcagatattcgagcgcatgagcaagggcggcttcaagggtgccttcgtgtgggggcagaacccctgtgcgggcggggccaacgcgggcaagaaccgcaaggctatggagacgctggactggatggtggtggtcaacctcttcgagaacgaaagttcgctcttctggaaggggccgggggtagaccccgccaaggtcaagaccgaggtgttcttcctgcctgcgtgcatgagcgtcgagaagggcggttccatcgccaattccggtcgctggctgcaatggcgtgaaccggggccgaaacccatgggcgacagccgttcggacggtgacatcgtcctcgacctctatgacgagatacgcaaactctaccgcgaggagaagggagccttccccgaacccgtgctggcgctggacaccgactaccgtaccgacggcaagtacgatcaccacaaggtggcgaagacgctgaacggcaagttcctcacggacgtgaccataggcgacaagacctacaaggcggggcagcaggtacccggcttcgccatgttgcaggccgacggttcgacgacgtccgggtgttggatattcaccgggtgctataccgacgccggtaacatgatggcgcgccgagaccgtacgcagaccccggaacaggccgccatagggctgttccccaactggtcgtatgcatggcccgccaaccgtcgcatcctgtacaaccgtgcggcggtggacatgaccggcaagcccttcgaccccaagcgtgccgtcatcgcatggaacggcgagaagtgggtgggcgacgtgcctgacggcggctggaagcccggagagaagttgcccttcatcatgatacgcgaggggcgcggtcagttgttcggccccggcagggtcgacgggcctttcccggagcactacgaaccgttcgagagtcctctcgaaagccatcccttctcgaagcagcgggtcaaccccactgcgctggcgttcagccacgaacccaaggcggtgcgcgacaagcgctaccccttcatctgcactacctaccgcgtcaccgaacagtggcagtcgggcacgatgacccgcaacaccgggtggctcaaggagatgcagccggagggcttctgcgagataagccgcgaactggccaaggaactcggcatcgccaacggcgacgccgtggtgctcgaatcgctgcggggcaaggtgcaggtggtcgccatcgtcacgccacgtctcaagcccttcaaggtcatgggcgaggtcatgcacgaggtgggcataccgtggcagttcggctgggggcagcatgtgggcaagggcgactctgccaacctgctctcgccttcggtgggcgaccccaacactggcattcccgagaccaaggtcttcatggtcaacctgcgcaaggcctagtggcgcgccatcagaagacttgatgcatgcatggcgcgataccgaccccttgcgggggcggtgtacgcggcaagaagtggagcggtcgcgccggagacgatgtctccggcgtgcgccccctcggcagcctcccgcagggaatgcggagagtgtcgaggcgtcacatcagggaggcatacatgaacggcaagacgttcttcatagaccagacgcgttgcacggcctgtcggggctgtcaggccgcctgcaagcagtggaagaagttcggcagcatcgagacgcgcaacaccggctcataccagaatccgcccgacctcgggccgagcaccttcaagctggtgcgcttcaacgaggtcgaggtggatggcaaactgaaatggctgttcttcccggagcaatgccgccactgtctcgaaccaccgtgcaagatggtggcggatgccctcattccgggtgccatcgtgcaggatgcggagaccggggcggtgctctacaccgagaagaccaagggactggactacatggaggtgcgctcggcgtgcccctacgacatcccgcggcaagacccggcaacgggtctgctcaccaaatgcaacatgtgcatcgaccgggtgcagaacggcatgctgcccgcgtgcgtgaagacctgccccacggggaccatgcacttcggtgacagggccgacatgcttgaactggcgcagaaacgcctcggcgaggtgaagaagaggagccccgcagccctgctggcggactccgaccttgtacgggtcctgtacctgtgcgagctcgcccccgccaactaccattcgcacatggtggcggaagccgacacctcgcgcatcgggcccttcagccgtcgcgccttgttgcgcctgcgtcctttcgcctag

(19) United States
(12) Patent Application Publication
(10) Pub. No.: US 2025/0357519 A1
(43) Pub. Date: Nov. 20, 2025

(54) APPLICATIONS OF O2-INSENSITIVE FORMATE DEHYDROGENASE

(71) Applicants: C. S. RAMAN, Baltimore, MD (US); Joel E. GRAHAM, Baltimore, MD (US)

(72) Inventors: C. S. RAMAN, Baltimore, MD (US); Joel E. GRAHAM, Baltimore, MD (US)

(73) Assignee: UNIVERSITY OF MARYLAND, BALTIMORE, Baltimore, MD (US)

(21) Appl. No.: 18/727,832

(22) PCT Filed: Jan. 10, 2023
(86) PCT No.: PCT/US2023/060407

§ 371 Date: Jul. 10, 2024

Related U.S. Application Data
(60) Provisional application No. 63/298,307, filed on Jan. 11, 2022.

(51) Int. Cl. H01M 8/16; C12N 9/02; C12N 11/02; C12P 3/00; H01M 4/90

(52) U.S. Cl. CPC H01M 8/16; C12N 9/001; C12N 9/0053; C12N 9/0061; C12N 9/0093; C12N 11/02; C12P 3/00; C12Y 103/03005; C12Y 109/03001; C12Y 110/03002; C12Y 117/01; H01M 4/90

(57) ABSTRACT

Disclosed are methods and apparatuses utilizing an O₂-insensitive FDH2 from the sulfate-reducing bacterium (SRB) Desulfovibrio vulgaris Hildenborough (DvH). The O₂-insensitive FDH2 may be applied to a biofuel cell for generating electricity and generating hydrogen peroxide. The biofuel cell can also be applied to wearable or implantable devices as a power source. The O₂-insensitive FDH2 can also be used in other applications not applying a fuel cell, such as hydrogen peroxide generation, a formate testing kit, or carbon capture applications.

### CROSS-REFERENCE TO RELATED APPLICATIONS

This application claims priority to U.S. Provisional Patent Application No. 63/298,307, filed on Jan. 11, 2022, the entire contents of which is herein incorporated by reference.

### STATEMENT REGARDING FEDERALLY SPONSORED RESEARCH OR DEVELOPMENT

This invention was made with government support under Grant Number DE-SC0018047 awarded by the Department of Energy. The government has certain rights in the invention.

### TECHNICAL FIELD

The disclosed embodiments relate to applications of an O₂-insensitive formate dehydrogenase, including a formate/air biofuel cell that does not require protection from O₂, and other various applications such as generation of electricity, kit and method for generation of hydrogen peroxide, kit and method for formate detection, device and method for carbon capture, and medical devices including the formate/air biofuel cell. None of the disclosed methods, kits or devices require protection from O₂.

### BACKGROUND OF INVENTION

The simplest carboxylic acid (formic acid), and its conjugate base (formate) are normal products of metabolic activity in living organisms, including bacteria and humans.¹ However, formate derived from human gut microbiota metabolism drives inflammatory dysbiosis² and progression of colorectal cancer.³ .² Although formic acid is primarily used as a food preservative (E236) or as silage additive for maintaining the nutritive value of animal feed,⁴ it is a highly sought-after electron-mediator and feedstock in (electro)microbial bioproduction,⁵ as well as a low carbon-footprint molecule that serves as a chemically robust hydrogen storage medium.^{6,7} In addition to being a carbon and energy source for the (an)aerobic growth of disparate bacteria,⁸ archaea,^{9,10} and syntrophic consortia,11 formate can be generated abiotically from CO₂ and renewable electricity.⁶

Formate oxidation and CO₂ reduction are interconvertible processes that are carried out by prokaryotic formate dehydrogenases (FDHs) (Reaction 1).^{12,13}

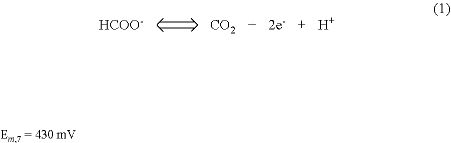

There are two phylogenetically distinct FDH families that can be distinguished by their transition metal ion requirement for enzyme activity.¹⁴ Metallo-FDHs are thought to be highly sensitive to O₂,^{15,16} necessitating catalytic measurements under anaerobic conditions. However, Desulfovibrio vulgaris Heldenborough (DvH)-FDH3 has been reproducibly shown to be O₂ sensitive^{17,18} while its ortholog from Desulfovibrio desulfuricans ATCC 27774 (Dd) can be purified in air.^{19,20} Similarly, Desulfovibrio gigas (Dg) FDH1 is readily isolated and stored under atmospheric conditions^{21,22} but its counterpart from DvH has been purified in the presence of 10 mM sodium nitrate and glycerol.²³ Independent of the procedures involved, the resulting enzymes are not fully active in that they must first undergo lengthy incubations with high concentrations of thiols (10-50 mM dithiothreitol²³ for DvH-FDH1 and 130 mM β-mercaptoethanol^{19,20,22} for Dd-FDH3 and Dg-FDH1) and/or formate^{20, 24} prior to catalytic measurements under anaerobic conditions. A representative example can be found in Figure S5a of Oliveira et al.,²³ where turnover numbers (TNs) continue to be an order of magnitude lower than the reported values even after reductive activation. Because the latter is specific to FDHs isolated from sulfate-reducing bacteria (SRB) and not utilized in other systems,²¹⁻²⁸ it is probable that the enzyme preparations constitute an admixture of inactive and active forms.²⁰ This is reminiscent of aerobically purified SRB [FeFe] hydrogenase (Hnd), which is also in an inactive state and requires preincubation with DTT or H2 to regain activity.²⁹ A molecular explanation for these observations has not been forthcoming.²¹

The situation is unclear with FDHs isolated from non-sulfate-reducing bacterium (non-SRB). Escherichia coli Fdh-H has been purified and characterized in the presence of sodium azide to minimize O₂ inactivation.^{27,30,31} 10 mM sodium nitrate^{26,32} or azide^{33,34} or ammonium sulfate and cysteine/DTT35-37 have been added as stabilizers during the isolation of other bacterial metallo-FDHs as well. Although azide is a transition-state analogue of formate,³⁸ very little is known about how azide and other small molecules protect the enzyme from O₂. Despite the aerobic stability of the biocatalysts, anaerobic conditions are essential for maintaining activity. Thus, the inhibitors are either removed prior to measurements under anaerobic conditions^{23,26} or allowed to remain while the activity is probed anaerobically^{31,39} or in air.⁴⁰

No FDH has been shown to reversibly interconvert formate and CO₂ in air, and mechanistic details regarding how O₂ reacts with these metalloenzymes are not available. As such, it is desired to obtain an FDH which can be utilized in aerobic and anaerobic conditions. Such an FDH could be useful in various applications, such as a formate/air biofuel cell, methods of generating electricity, kit and methods of generating hydrogen peroxide, kit and method of detecting formate, and device and methods of carbon capture. Although formate/air biofuel cells including an FDH have been developed, such prior formate/air biofuel cells include an O₂-sensitive FDH, and therefore require O₂ protection by a redox polymer gel, for example.

### BRIEF SUMMARY OF INVENTION

The disclosed embodiments take advantage of the discovery that a particular formate dehydrogenase is O₂-insensitive. Present-day claims of FDH O₂ sensitivity fail to recognize or rationalize previously reported findings regarding the existence of metallo-FDHs capable of oxidizing formate with oxygen (Reaction 2).

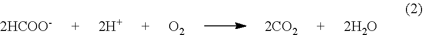

Starting with the first purification of a bacterial FDH, O₂ uptake served as a proxy for measuring enzyme activity.⁴¹ Subsequently, Escherichia coli hydrogenlyase (Fdh-H) was isolated, revealing that it was not responsible for the formate oxidase (FOX) activity.⁴² It is also known that formate dependent O₂ consumption by E. coli is higher in aerobically grown cells.⁴³ Additionally, it has been shown using an O₂ utilization assay that not only E. coli FDH requires molybdenum and selenium for function, but more importantly, that it retained considerable FOX activity.⁴⁴ This has been confirmed by several research laboratories.⁴⁵⁴⁷ It has also been shown that FOX activity was broadly distributed across bacteria.⁵⁰ Further, the third Fdh-O (O for oxidase; the remaining two being Fdh-N (nitrate)⁵¹ and Fdh-H (hydrogen)⁵²) in E. coli have been discovered.^{12,53,54} Although others have confirmed the presence of Fdh-O,^{55,56} isolation and characterization of a metallo-FDH with FOX activity has proven to be difficult.⁵⁷ The possibility that coexistence of dehydrogenase and oxidase activities would render a metallo-FDH insensitive to O₂, by reducing the latter to harmless products, has not been entertained thus far.

However, it is herein shown that FDHs capable of transferring electrons to natural high potential acceptors are likely to be O₂-insensitive by virtue of their FOX activity, for such physiological reactions are poised to occur under aerobic conditions. Despite the paucity of information regarding redox partners (two well characterized systems exhibit low reduction potentials^{21,58}), the herein disclosed embodiments were inspired by the observation that an FDH from D. vulgaris Miyazaki (DvM) preferentially transfers electrons to a high-potential cytochrome c₅₅₃.^{59,60} Because the genetically tractable DvH^{61,62} is closely related to DvM,⁶³ thrives in microaerobic niches,⁶⁴⁻⁶⁶ and encodes a 73% identical cytochrome c₅₅₃ (E_{m,7}=+62 mV),⁶⁷ the O₂ sensitivity of periplasmic FDHs was probed.⁵³⁻⁵⁵ The poorly characterized DvH-FDH2 (locus tag DVU2482-2481)^{68,69} and cytochrome c₅₅₃-reducing DvH-FDH3 (locus tag DVU2812-2809)^{17,18} was studied instead of the well-studied DvH-FDH1 (locus tag DVU0586-0588)²³, which couples anaerobic formate oxidation to sulfate reduction by initiating electron transfer to a low-potential cytochrome c₃ (E_{m,7}=−350 mV).⁶² Here, discovery and characterization of an O₂-insensitive FDH that retains both formate dehydrogenase and oxidase activities is described.

The O₂-insensitive FDH can be applied for several practical uses. First, the O₂-insensitive FDH can be used in a biofuel cell which utilizes formate and air to generate electricity, without requiring protection from O₂. The O₂-insensitive FDH can also be used in a wearable or implantable medical device in order to generate electricity. Such devices include, for example, a contact lens and a pacemaker. The O₂-insensitive FDH can also be applied to a method of generating electricity. The O₂-insensitive FDH can also be applied to a kit and method of generating hydrogen peroxide, particularly in situations where the carriage or storage of hydrogen peroxide is untenable due to reactivity limitations. Additionally, the O₂-insensitive FDH can also be applied to a kit and method of formate detection, which eliminates the need for an expensive NAD cofactor, and allows for detection of formate where NAD/NADH would interfere in a standard kit. Such a formate detection kit could measure formate levels in the gut, soil, or seawater for example. Also, the O₂-insensitive FDH can be applied to a device which serves as a safety indicator in the manufacture of methanol or chemical with reactive methyl groups, because the formate metabolite would rise with exposure. Furthermore, the O₂-insensitive FDH can also be applied to a device and method for carbon capture, to convert CO₂ in the air (direct air capture) or remove CO₂ resulting from burning coal, gas, oil, or biomass prior to atmospheric release, or indirectly capture CO₂ from seawater, all producing stable formate. This O₂-insensitive FDH is notable in that it is functional in both aerobic and anaerobic environments, and does not require any redox polymer protection.

Additionally, applications include detection of in situ formate levels in colorectal cancer, through the swallowing of a capsule, which would also electronically report back the levels of formate in the gut, as well as artificial photosynthesis in a wireless device which makes clean fuel from sunlight, CO₂ and water (gasworld.com).

### BRIEF DESCRIPTION OF DRAWINGS

FIG. 1. Structure and function of FDH operons in DvH. Panel A shows a condensed map of the three fdh loci. fdnG2 (yellow; large subunit) and fdnH2 (magenta; small subunit) encode FDH2 and are part of a five gene operon. Short intergenic regions are illustrated at the nucleotide level, while the length of their long counterparts is identified by two- or three-digit numbers. Periplasmic FDH localization is made possible by the twin-arginine translocation (Tat) signal peptide (cyan). Theoretical molecular masses of the encoded polypeptide in daltons are listed below each gene. Panel B shows anaerobic growth curves of JW2127: formate-acetate-sulfate (red), lactate-sulfate (blue). The lines going through the points represent fits to Weibull⁷⁰ growth model. Error bars represent standard deviations from three biological replicates.

FIG. 2. Isolation of DvH-FDH2. Panel A shows streamlined expression and purification workflow. Single colonies resulting from the transformation of fdh2 plasmid into strain JW2127 were used to start a pre-culture that served as the inoculum for a 10 L scaleup, cells from which were aerobically lysed and subjected to affinity purification, yielding StrepII-tagged FDH2. Panel B shows SDS-PAGE of purified protein (lane 1) and molecular weight markers (lane M). α and β represent the large and small subunits of FDH2, respectively. Panel C shows that following non-denaturing PAGE, FDH2 activity (dark single band) is detectable in air via NBT staining.

FIG. 3. Electronic spectra of DvH-FDH2. As-isolated (blue), formate-reduced (green), and dithionite-reduced (orange) states are shown in panels A and C. Difference spectra are shown in panels B and D. As-isolated minus formate-reduced and as-isolated minus dithionite-reduced are in green and orange, respectively. Formate-reduced difference spectra (green) resemble those reported for E. coli Fdh-H.78 Conditions: 50 mM Tris-HCl pH 8, 7 μM enzyme, 10 mM formate, or 10-30 μM dithionite

FIG. 4. Electronic and Electron Paramagnetic Resonance (EPR) spectra of DvH-Fdh2. Panel A shows (i), (v) as isolated (45 μM). (ii), (vi) dithionite-reduced (45 μM). Red arrows indicate approximate location of W^{V} g tensors. (iii), (vii) formate-reduced aerobic (45 μM). (iv), (viii) formate-reduced anaerobic (27 μM). (i)-(iv) were collected with 10 Gauss modulation amplitude and 0.2 mW power at 15K. (v)-(viii) were collected with 10 Gauss modulation amplitude and 4 mW power at 26K. Panel B shows simulation of the W center of dithionite-reduced sample. (i) W^{V} EPR spectrum (black trace) and simulation (red trace) of 150 μM DvH-Fdh2 collected with 8 Gauss modulation amplitude and 10 mW power at 108K. (ii), (iii) Scaled individual contributions to the simulation of the composite spectrum in (i). Simulation includes hyperfine splittings originating from the 14.3% naturally occurring ¹⁸³W (I=½). Simulation parameters are presented in Table 2.

FIG. 5. Full progress curve probing of DvH-FDH2 catalysis. Unmodified raw experimental traces are shown in panels A (BV), D (PES/DCPIP), and G (PES/DCPIP). Arrows represent the point at which the experiments were started by either the addition of formate (panel A) or FDH2 (panels D and G). Data normalized for electron acceptor concentration (panels B, E, and H) were globally fit (solid lines) using Kintek Explorer. Whereas panel B was fit to model shown in Scheme S1, panels E and H were fit to the counterpart in Scheme 1. Confidence contour analysis (panels C, F, and I) illustrates that both k₊₁ (k_{cat}/Kₘ) and k₊₂ (k_{cat}) are well constrained by the kinetic data. Upper and lower bounds of each rate constant is reflected by the axes labels. When plotted as a function of one another, red ovals signify the extent of variability in k₊₁ and k₊₂ while still being constrained by the model. Consequently, both parameters display a defined boundary (red), χ² of which is 0.95 (side bar). Table 5 lists rate constants, as well as best fit parameters derived from this analysis.

FIG. 6. Product generated by DvH-FDH2 catalyzed reaction. Panel A shows ¹³C NMR spectra at pH 7.5. Panel B shows ¹³C NMR spectra at pH 6. ¹³C-formate (bottom), ¹³C-formate+enzyme (middle), ¹³C-formate+enzyme+PES (top).

FIG. 7. Mechanistic basis of O₂ insensitivity. Panel A shows that oximetry reveals coupling of formate oxidation to O₂ reduction. Panel B shows oximetry in the presence of catalase. Panel C shows a comparison of rates of O₂ uptake in the absence (N=15) and presence (N=10) of catalase. Both dot and box plots are shown with the latter deemphasized due to the medium sample size.⁹⁷ Panel D shows enzymatic H₂O₂ generation monitored via the AR assay (filled red circles) (n=3). Panel E shows enzymatic H₂O₂ production evaluated using the CBA assay (filled blue circles) (n=3). Filled orange circles represent data obtained at 10 nM FDH2. Filled green circles denote H₂O₂ formation by DvH-FDH2 in the presence of 30 μM equine cytochrome c (n=3). In both panels D and E, open gray circles represent the H₂O₂ standard curve determined in the absence of formate or FDH2. Panel F shows a reduction of equine cytochrome c under aerobic (blue) and anaerobic (red) conditions. N=3. Points of addition of formate (F), enzyme (E), catalase (C), heat denatured enzyme (E_{D}), H₂O₂.(H) are identified by down arrows. u, v, x, and y are defined in herein.

FIG. 8. Molecular insights into the O₂ insensitivity of DvH-FDH2. Panel A shows AlphaFold-based tertiary topology illustrating the regions that are different between FDH1 and FDH2 in purple. The four [4Fe-4S] clusters are depicted by a combination of yellow and orange spheres. Tungstopterin is shown at the top in blue and red spheres. Panel B shows a closeup view of the [4Fe-4S] cluster found in the large subunit.

FIG. 9. Panel A shows growth curves of JW2111. Panel B shows growth curves of JW2121. Blue and red traces represent growth on MOYLS4 and MOYFAS4, respectively. FDH1 is the sole FDH encoded by JW2111 and is able to support growth on MOYFAS4. However, JW2121, which lacks both FDH1 and FDH3, does not. In the MOYFAS4 medium, 60 mM formate and 10 mM acetate are present to support growth but lactate is excluded.

FIG. 10. Nitroblue tetrazolium chloride (NBT)-strip assay reveals aerobic formate oxidation by DvH-FDH2. The ability of the latter to transfer electrons to NBT is retained both in the presence and absence of phenazine methosulfate (PMS). A blue spot develops within 15 s only when enzyme, formate, and NBT are mixed. Although inclusion of PMS accelerates spot formation, it is not essential for the assay to succeed.

FIG. 11A. Sequence alignment of the large R subunit of DvH-FDH2 (DVU2482) (top, SEQ ID NO: 31) with its DvH-FDH1 counterpart (DVU0587) (bottom, SEQ ID NO: 29). Residues underlined in red represent the Tat (twin arginine translocation pathway) signal peptide (predicted by SignalP 6.0),₉ which is cleaved off by a peptidase (Tat/SPase I) following export to periplasmic space. Cys residues coordinating the single [4Fe-4S] cluster are identified by §. X identifies amino acids that are uniquely different between the two subunits. Sec is denoted by U.

FIG. 11B. Sequence alignment of the small a subunit of DvH-FDH2 (DVU2481) (top, SEQ ID NO: 32) with its DvH-FDH1 counterpart (DVU0588) (bottom, SEQ ID NO: 30). Cys residues coordinating the three [4Fe-4S] clusters are identified by §.

FIG. 12. Comparison of the electronic spectra of DvH-FDH2 and DvH-FDH1²³.

FIG. 13. Simulation of Fe/S centers of formate-reduced FDH2 prepared under aerobic conditions. (i) EPR spectrum (from FIG. 4(iii)) and simulation (red trace). (ii), (iii) Scaled individual contributions to the simulation of the composite spectrum in (i). Simulation parameters are presented in Table 2.

FIG. 14. Validation Scheme S1 using source²⁰ BV kinetics data from Dd-FDH3. Panel (A) shows Raw traces (points) and the associated global fits (solid lines). Panel (B) shows confidence contour analysis (for details on how to interpret this plot, see legend to FIG. 5). Panel (C) shows classical Michaelis-Menten analysis. Panel (D) shows fitting the data in panel (C) to extract k_{cat} and k_{cat}/Kₘ (k_{SP}) using the approach described by Johnson.⁸⁴ Initial velocities were obtained using ICEKAT.¹⁸⁸ Panel (E) shows comparison of kinetic parameters derived from three different approaches.

FIG. 15. Validating Scheme S1 with DvH-FDH1 source²³ data on BV reduction. Panel (A) shows Raw kinetic traces of BV reduction. Panel (B) shows Full progress curves (points) extracted from panel A for global fitting analysis. Fits are shown as solid lines. Panel (C) shows Confidence contour analysis. Panel (D) shows nonlinear regression of Michaelis-Menten equation. Panel (E) shows fitting the initial velocity data according to Johnson.⁸⁴ Panel (F) shows a summary of the results.

FIG. 16. Reduction of 2 mM BV by DvH-FDH2 as a function of formate concentration (0.5-60 uM). Panel (A) illustrates raw kinetic traces, Panel (B) illustrates controls using no formate (grey) and no enzyme (blue), Panel (C) illustrates a nonlinear regression of the Michaelis-Mentin equation, Panel (D) illustrates a nonlinear regression using the equation of Johnston (2019). Error bars represent standard deviation of three independent measurements.

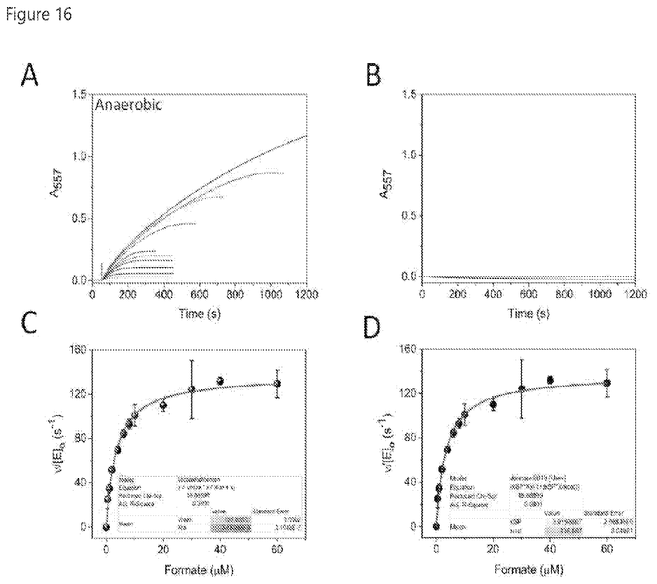

FIG. 17. DvH-FDH2 catalyzed reduction of 20 mM BV as a function of formate concentration. Panel (A) illustrates raw kinetic traces alongside endpoint values, Panel (B) illustrates concentration normalized traces, Panel (C) illustrates controls using no formate (grey) and no enzyme (blue), Panel (D) illustrates a nonlinear regression using the Michaelis-Mentin equation, Panel (E) illustrates a nonlinear regression using the equation of Johnson (2019), and Panel (F) illustrates a determination of BV extinction coefficient from full progress curve endpoints. Plotting the latter values as a function of [formate] results in a slope, which after correcting for 2BV⁺:1F stoichiometry, yields a value of 12,089±38M⁻¹ cm⁻¹. This is yet another way to validate stoichiometric reduction of BV by DvH-FDH2. This approach to the determination of BV extinction coefficient is similar to that employed using hydrogenase as an electron donor.²¹² Literature molar extinction values for BV range from 7.0-19.5 mM⁻¹ cm⁻¹ depending on the wavelength of measurement (546, 555, 578, 580 or 600 nm)^{23,20,212-217,30218,219,19,220,71}. Because source kinetics data involving artificial electron acceptors is seldom reported in the literature, it is not possible to properly validate the kinetic parameters and/or reaction stoichiometry of a metallo-FDH. When combined with the fact that the reaction product is also rarely measured, the results turn out to be largely phenomenological and lack information content essential for making advances in the field.

FIG. 18. Glucose oxidase does not interfere with BV reduction by DvH-FDH2. Panel (A) illustrates BV reduction by DvH-FDH2 in the absence (blue) or presence (red) of 1 U/mL glucose oxidase, and Panel (B) illustrates a close up of the initial rate (55-65 s) used for initial velocity via ICEKAT¹⁸⁸.

FIG. 19. Optimizing experimental conditions for PES/DCPIP reduction in air. Panel (A) illustrates that DvH-FDH2 does not transfer electrons to DCPIP in the absence of PES (cyan), and control with both DCPIP and PES (pink), with an inset showing an initial part of trace enlarged. Panel (B) illustrates varying DCPIP while maintaining fixed PES where solid lines are anaerobic and dotted lines are aerobic. Panel (C) illustrates varying PES while maintaining constant DCPIP. Panel (D) illustrates final optimization. The magenta trace was highly reproducible and the upward sloping baseline region is not an artifact. It occurs under conditions where the concentration of formate is in the vicinity of [DCPIP]. Panels (E) and (F) each illustrates controls using no formate (blue) and no enzyme (grey).

FIG. 20. PES/DCPIP reduction by DvH-FDH2 in air. Panel (A) illustrates controls with no enzyme (grey) and no formate (blue). Panel (B) illustrates raw kinetic traces as a function of varying formate concentration. DvH-FDH2, DCPIP, and PES levels were fixed. Panel (C) illustrates concentration-normalized and inverted (product increases as a function of time) traces. Panel (D) illustrates a non-linear least squares fit to the Michaelis-Mentin equation, Panel (E) illustrates a fit to the equation of Johnson⁸⁴ for extracting k_{cat} and k_{cat}/Kₘ. Initial velocities were obtained via ICEKAT.¹⁸⁸ and Panel (F) demonstrates that enzyme activity is not lost after plateauing. Addition of a second formate aliquot (second up arrow) restores the original progress curve. When possible, this approach is superior to the Selwyn test,²²¹ which requires progress curve measurements at different [enzyme] but fixed [substrate]. Unfortunately, the Selwyn test lacks the ability to offer insights into the inactivation rates or the extent of enzyme inactivation.

FIG. 21. PES/DCPIP reduction by DvH-FDH2 under anaerobic conditions. Panel (A) illustrates controls with no enzyme (grey) and no formate (blue). Panel (B) illustrates raw kinetic traces as a function of varying formate concentration. DvH-FDH2, DCPIP, and PES are fixed. Panel (C) illustrates concentration normalized and inverted traces, Panel (D) illustrates a non-linear least squares fit to the classical Michaelis-Mentin equation, Panel e Fit to Johnson's equation⁸⁴ for extracting k_{cat} and k_{cat}/Kₘ. Initial velocities were obtained via ICEKAT188. Fit parameters are included within the plots. Panel (F) illustrates spectral changes associated with DCPIP reduction. Absorbance at 600 nm (down arrow) decreases as DCPIP is reduced.

FIG. 22. Effect of sodium azide on PES/DCPIP reduction by DvH-FDH2. Panel (A) illustrates raw anaerobic kinetics, Panel (B) illustrates data from Panel (A) converted to reduced DCPIP concentration, Panel (C) illustrates 4-parameter sigmoidal fit of Panel (B), Panel (D) illustrates aerobic raw traces, Panel (E) illustrates data from Panel (D) converted to reduced DCPIP concentration, and Panel (F) illustrates a 4-parameter sigmoidal fit of Panel (C). IC50 values were in the range of 0.8 mM.

FIG. 23. Effect of sodium nitrate on PES/DCPTP reduction by DvH-FDH2. Panel (A) illustrates raw anaerobic kinetics, Panel (B) illustrates data from Panel (A) converted to reduced DCPTP concentration, Panel (C) illustrates raw aerobic kinetics, and Panel (D) illustrates data from Panel (C) converted to reduced DCPTP concentration.

FIG. 24. ¹³C NMR spectrum of ¹³C-formate+DvH-FDH2 at pH 7.5 showing production of C-13 bicarbonate.

FIG. 25. ¹³C NMR spectrum of ¹³C-formate+DvH-FDH2+PES at pH 7.5 showing higher production of bicarbonate in the presence of electron acceptor.

FIG. 26. ¹³C NMR spectrum of ¹³C-formate+DvH-FDH2 at pH 6.0 showing production of C-13 bicarbonate and C-13 CO2.

FIG. 27. ¹³C NMR spectrum of ¹³C-formate+DvH-FDH2+PES at pH 6 showing higher production of C-13 bicarbonate and C-13 CO2 in the presence of electron acceptor.

FIG. 28. ¹³C NMR spectrum of isotopically enriched (99%) ¹³C-sodium bicarbonate at pH 6.

FIG. 29. ¹³C NMR spectrum of isotopically enriched (99%) ¹³C-formate at pH 6.0.

FIG. 30. ¹³C NMR spectrum of isotopically enriched (99%) ¹³C-formate at pH 7.5.

FIG. 31. ¹H NMR spectrum of isotopically enriched (99%) ¹³C-formate at pH 6. The formyl singlet splits into a doublet in the 1-H spectrum due to the coupling of H-1 C-13 (j˜195 Hz). 1% C-12 formate is visible as a singlet (8.47 ppm) sandwiched between the doublet.

FIG. 32. Graph showing that DvH-FDH2 lacks catalase activity. Panel (A) shows that upon incubation with H₂O₂(H) and the enzyme (E), no O₂ evolution occurs. Subsequent addition of catalase (C) led to O₂ production, which was enhanced by further (H) addition. Addition of formate resulted in consumption of O₂ despite the high (˜32%) O2 concentration. Panel (B) illustrates that heat-denatured FDH2 failed to consume O₂, since further addition of catalase did not produce a response. To prove that exogenously added catalase was still functional, H₂O₂ was added, resulting in O₂ production.

FIG. 33. Graph showing that O₂ uptake by DvH-FDH2 remains unaltered in the presence of SOD. DvH-FDH2 consumes oxygen in the presence of formate, and addition of catalase results in a burst of oxygen production which is then consumed by further turnover.

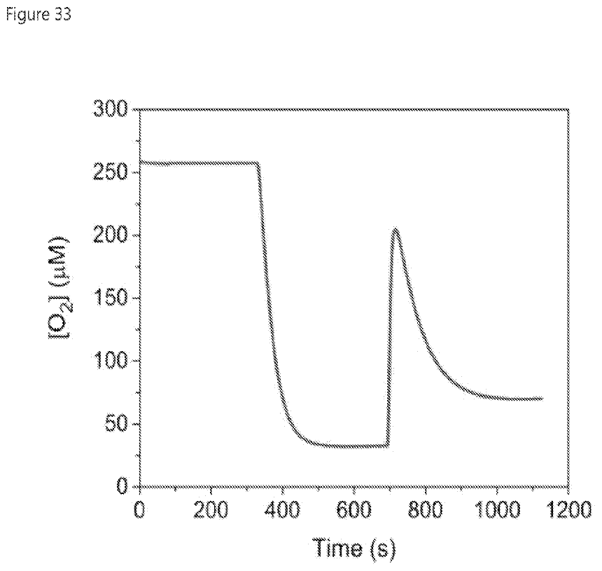

FIG. 34. Direct reduction of equine cytochrome c during aerobic DvH-FDH2 catalysis. Electronic spectra of: Oxidized cytochrome c (blue), FDH2+cytochrome c (cyan), FDH2+cytochrome c+formate (green), FDH2+cytochrome c+2× formate (violet), FDH2+cytochrome c+2× formate+dithionite (orange). Q-band and near-infrared region of the spectra have been magnified 5× to reveal the 695 nm band, which is a direct indicator of the functional integrity of cytochrome c. Photometric range of the Shimadzu UV-2600i spectrophotometer used in this measurement is ±5 absorbance units.

FIG. 35. Graph showing that acetylated equine cytochrome is not reduced during DvH-FDH2 catalysis. DvH-FDH2 does not reduce acetylated cytochrome c, indicating that protein-protein interactions are required and cytochrome C reduction is not spurious.

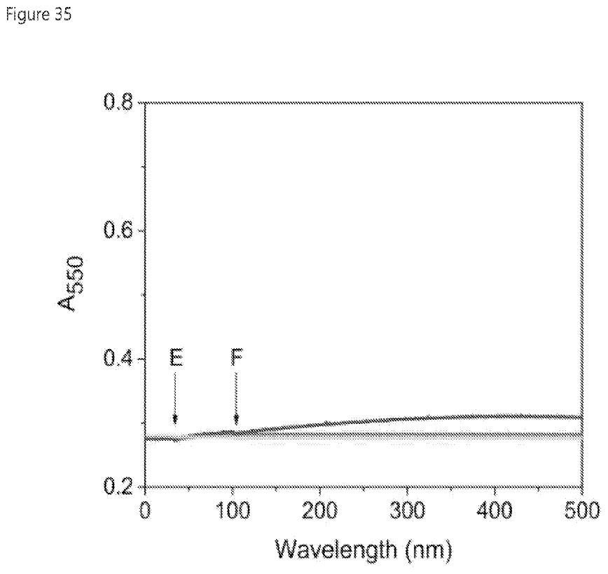

FIG. 36. Experimental workflow for facile medium scale (10 L) cultivation of DvH. To a 10 L carboy containing pre-warmed sterile MOYLS4 medium, the following were added in sequence using a sterile syringe: vitamins, iron chloride/EDTA solution, and spectinomycin. The carboy lid was closed tightly. Carboy was gassed with N2 through stopper to remove air in the headspace; vented with a 23 gauge needle (Panel A). Sodium sulfide was injected via sterile anaerobic syringe transfer (Panel B). Carboy was mixed by rolling or shaking and subsequently incubated at 37° C. until resazurin turned colorless. Carboy was connected to a 500 mL culture bottle using a transfer line fitted with 18-gauge needle. Transferred 250-500 mL of active culture to carboy while venting with a 23-gauge needle (Panel C). Incubated at 37° C. Monitored growth via OD550 until it plateaued and, finally, chilled the carboy on ice (Panel D).

FIG. 37. Experimental workflow for BV assay. Experimental workflow for BV assay. Setup cuvette (1), gas with argon (2), fill with reaction mix (3), inject GO and catalase (4), gas with argon while mixing for 20 minutes (5), transfer to spectrophotometer chamber and continue stirring (7), initiate a kinetics run by injecting formate (8), continue data collection until the reaction goes to completion (9). Blue arrows shown at the bottom in steps 5 and 7 to 9 denote stirring.

FIG. 38. Experimental workflow for aerobic PES/DCPIP assay. Prepare cuvette (1), fill with reaction mix (2), add stirrer (3), initiate kinetics by the addition of DvH-FDH2 while the contents of the cuvette are being mixed (4), and continue data collection until completion (5). Blue arrows shown at the bottom in steps 4 and 5 denote stirring.

FIG. 39. Experimental workflow for anaerobic PES/DCPIP assay. Setup cuvette (1,2), add reaction mix (3), gas with argon (4), start the reaction by injecting DvH-FDH2 (5), and collect data until the reaction goes to completion (6). Blue arrows shown at the bottom in steps 4-6 represent stirring.

FIG. 40. Cyclic voltammetry charts (CVs) for formate oxidation (left) and formate/CO₂ interconversion (right). FDH was immobilized and tested under aerobic conditions in 50 mM Tris-HCl buffer, pH 7.0. Scan rate: 5 mV s⁻¹.

FIG. 41. Representative polarization curve (black line) and power curve (red line) for the formate/O₂ biofuel cell. The cathode chamber with laccase bioelectrode was saturated with O₂ in a citrate/phosphate buffer, pH 4.0. The anode chamber with FDH bioelectrode was exposed to the air in 50 mM Tris-HCl buffer, pH 8.0. Experiments were conducted with linear sweep polarization at 0.5 mV s⁻¹. Open Circuit Potential: 1.17±0.02 V; Jₘₐₓ: 668±77 μA cm⁻²; Pₘₐₓ: 250±6 μW cm⁻²

FIG. 42. CVs of FDH/BV-LPEI in 50 mM Tris-HCl buffer, pH 8.0; scan rate: 5 mV s⁻¹ (left). Polarization and power curves for formate/O₂ fuel cell. Experiments were conducted with linear sweep polarization at 0.5 mV s⁻¹ (right). The cathode chamber with laccase bioelectrode was saturated with O₂ in a citrate/phosphate buffer, pH 4.0. The anode chamber with FDH bioelectrode is in 50 mM Tris-HCl buffer, pH 8.0. OCP: 1.41±0.03 V; Jₘₐₓ: 473±18 μA cm⁻²; Pₘₐₓ: 181±12 μW cm⁻². FDH/BV-LPEI was immobilized and tested under anaerobic conditions.

FIG. 43. CVs of FDH/BV-LPEI (left) and polarization and power curves (right) for formate/O² fuel cell OCP: 1.34±0.01 V; Jₘₐₓ: 290±61 μA cm⁻²; Pₘₐₓ: 132±9 μW cm⁻². FDH/BV-LPEI was immobilized under anaerobic conditions but tested in open air. Other test conditions were the same as FIG. 42.

FIG. 44. CVs with FDH/NQ-LPEI immobilized and tested under anaerobic conditions (Panel A). CVs with FDH/NQ-LPEI immobilized and tested under air (panel B). Experiments were conducted in 50 mM Tris-HCl buffer, pH 8.0 with scan rate of 5 mV s⁻¹.

FIG. 45. CVs with FDH/Cc-PAA immobilized and tested under anaerobic conditions. Experiments were conducted in 1 M potassium phosphate buffer, pH 6.0; scan rate: 5 mV s⁻¹.

FIG. 46. CVs with FDH in solution (0.12 mg/mL) with 150 μM PMS under anaerobic and aerobic conditions. Experiments were conducted in 50 mM Tris-HCl buffer, pH 8.0; scan rate: 5 mV s⁻¹.

FIG. 47. CVs with FDH in solution (0.12 mg/mL) with 150 μM ferrocenium hexafluorophosphate under anaerobic and aerobic conditions. Experiments were conducted in 50 mM Tris-HCl buffer, pH 8.0; scan rate: 5 mV s⁻¹.

FIG. 48. CVs of FDH on HOPG activity inhibited by different concentrations of sodium nitrate. Experiments were conducted in 50 mM Tris-HCl buffer, pH 8.0; scan rate: 5 mV s⁻¹. FDH/HOPG was immobilized under aerobic conditions but tested anaerobically.

FIG. 49. A simplified structure of a biofuel cell as discussed herein.

FIG. 50. The structure of a five-gene operon including the FDH2. In the figure, theoretical molecular weights are shown at the bottom. Locus tags of the five genes are shown at the top (DVU2485-DVU2481). The number “110” above the intergenic region separating the two FDH subunits represents the number of nucleotides. The phrase “17 TMS” refers to integral membrane proteins that contain a total of 17 transmembrane segments.

FIG. 51. Working model of aerobic redox bifurcation by DvH-FDH2, combining combines two exergonic branches analogous to aerobic FBEB. Since Uhc is most likely the natural redox partner of DvH-FDH2, it is shown at the end of the bottom branch. Based on the ability to reduce Uhc by ascorbate (10 mM; Em,7=+90 mV)/TMPD (2 mM; Em,7=+240 mV) couple, it is anticipated that its Em,7≥+100 mV. Conditions: Reaction volume, 1 mL. 50 mM sodium phosphate buffer pH 7.5, 150 mM NaCl, 1 mM EDTA, 0.05% dodecyl maltoside, superoxide dismutase (50 μL of 10 mg mL-1) and catalase (12.5 μL of 10 mg mL-1).

FIG. 52. Molecular insights into the O₂ insensitivity of DvH-FDH2. Panel (A) shows AlphaFold2-based tertiary topology and quaternary structure. Shading in pink represents sites that are unique to FDH2. The four [4Fe-4S] clusters are depicted by a combination of yellow and orange spheres. The two pterin moieties coordinating Wco are shown at the top using blue and red spheres. Cofactors were docked manually into the predicted structure. Panel (B) shows variability within a 10 Å radius of W. Side chains belonging to DvH-FDH1 are shown in gray. W and sulfide are identified as blue and yellow spheres, respectively. For clarity, conserved sites are not shown (see FIG. 63). Panel (C) shows Environment of the large subunit [4Fe-4S] cluster (yellow-orange sticks). In Panels (B) and (C), bolded labels signify variations.

FIG. 53. Amplex red assay at a fixed concentration (5 μM) of formate (F) (n=3). Abscissa refers to sample numbers. H₂O₂ standard (5 μM) (black), (2) FDH2+F (green), FDH2+F+SOD (10 U/mL) (violet), FDH2+F+catalase (100 U/mL) (cyan), heat denatured FDH2+F (red), FDH2 alone (brown), and FDH2+F without HRP (blue). Unless specified otherwise, all samples contained buffer, DTPA, amplex red, and HRP. 1.6 nM FDH2 was used.

FIG. 54. CBA assay at a fixed (10 μM) concentration of formate (F) (n=3). Panel (A) Abscissa refers to sample numbers. H₂O₂ standard (10 μM) (black), FDH2+F (green), FDH2+F+SOD (10 U/mL) (violet), FDH2+F+catalase (100 U/mL) (cyan), heat denatured FDH2+F (red), FDH2 alone (brown), and FDH2+F without CBA (blue). 1.6 nM FDH2 was used. Unless specified otherwise, all samples contained buffer, DTPA, and CBA. Panel (B) shows electronic spectra of CBA (grey) and the product (7-hydroxy-coumarin, COH) (blue) derived from reacting CBA with 200 μM H₂O₂. Panel (C) is a representative image of a CBA assay plate under UV light.

FIG. 55. Rate of O₂ uptake by DvH-FDH2 remains largely unaffected by the presence of SOD. Purple and yellow traces were measured with and without SOD, respectively. E, enzyme, F, formate, and S, SOD.

FIG. 56. Partially acetylated equine cytochrome c is not significantly reduced during aerobic DvH-FDH2 catalysis. Points of addition of enzyme (E) and formate (F) are identified by arrows. Dithionite (D) addition at the end of the experiment is also shown as reference. Panel (A): E+F+cytochrome c (blue), E+cytochrome c (no formate control) (green). Panel (B) is closeup view of panel A, Panel (C) illustrates electronic spectra of oxidized (blue) and reduced (red) acetylated cytochrome c. A₅₅₀ refers to absorbance at 550 nm.

FIG. 57. Kinetics of cytochrome c reduction by FDH2. Panel (A) is an enlarged view of FIG. 7F depicting the early stages of the reaction. Aerobic (blue) and anaerobic (red) conditions are shown. Green/cyan dots represent the region used in estimating the initial rates. Panel (B) illustrates no formate (green) and no FDH2 (cyan) controls.

FIG. 58. Effect of SOD and catalase on the aerobic kinetics of native equine cytochrome c reduction by FDH2. All reactions were performed in 50 mM Tris-HCl buffer pH 8 and 30 μM cytochrome c. Enzyme (E; 1.6 nM) or formate (F; 10 μM) additions are shown by down arrows. Panel (A): SOD (10 U/mL) (solid violet), catalase (40 U/mL) (solid cyan), control without SOD and catalase (solid black), SOD (100 U/mL) (dotted violet), catalase (400 U/mL) (dotted cyan). Panel (B) is an enlarged view of panel A. There was a small absorbance change at 550 nm upon mixing FDH2 and oxidized cytochrome c. This was also visible in FIGS. 57 and 34.

FIG. 59. Panel (A): Confidence metrics associated with AlphaFold2.1 structure prediction. The intersecting lines in Panel (B) is a consequence of protein boundaries introduced by the use of two independent sequences (large and small subunits of FDH2) for predicting the structure of FDH2 heterodimer. pLDDT spike near residue 1000 (left panel) is also a result of the same.

FIG. 60. Backbone RMSD variations between the large subunits of FDH2 (DVU2482) and FDH1 (6sdv:A) at the single residue level.

FIG. 61. Backbone RMSD variations between the small subunits of FDH2 (DVU2481) and FDH1 (6sdv:B) at the single residue level.

FIG. 62. Difference residue-residue distance maps of FDH2:FDH1 large subunit pair. Zero (black), positive (cyan), and negative (yellow) differences are shown.

FIG. 63. Structural comparison of DvH-FDH2 and DvH-FDH1 heterodimers. Panel (A) shows superposition of the two proteins. FDH1 is in grey. Large and small subunits of FDH2 are shown in green and olive, respectively. Panel (B) shown overlay of the invariant active site residues. A bond between Sec191 and W in DvH-FDH2 is not shown for clarity.

FIG. 64. Assessing electron acceptor specificity of DvH-FDH2. Left and right cuvettes represent reaction mixtures before and after catalysis, respectively. Conditions: Total reaction volume of 3 mL, 50 mM Tris-HCl pH 8: BV: 5 mM BV, 3 mM sodium formate, and the reaction started by adding 25 nM FDH2; PES/DCPIP: 1 mM PES, 80 μM DCPIP, 100 μM sodium formate, and the reaction started by adding 25 nM FDH2; mPMS/WST-1: 75 μM mPMS, 100 M WST-1, 100 μM sodium formate, and the reaction started by adding 1.6 nM FDH2; Equine Cyt c: Reaction conditions are identical to that reported in FIG. 7F; and Potassium ferricyanide: 1 mM potassium hexacyanoferrate(III), 3 mM sodium formate, and the reaction started by adding 25 nM FDH2.

FIG. 65. A simplified structure of an air-sensitive biofuel cell for simultaneous electricity and hydrogen peroxide generation, having an anaerobic anode and aerobide cathode. Half of the cells are in relative isolation and only connected by a membrane, salt bridge or frit.

### DETAILED DESCRIPTION OF THE INVENTION

### I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “O2-insensitive” and similar phrases refer to an enzyme which maintains its enzymatic functionality in the presence of a gaseous environment of up to 42% O₂.

### II. The Present Invention

As will be discussed herein, the invention relates to an O₂ insensitive FDH and its various applications. The DvH-FDH2 is described herein (sometimes referred to simply as “FDH”) has a first subunit represented by SEQ ID NO: 31 and a second subunit represented by SEQ ID NO: 32. However, the FDH is not limited to this. Rather, an FDH may be utilized which has one or more additions, deletions, or substitutions relative to SEQ ID NOs: 31 and 32. For instance, the first and second FDH subunits may each have 90%, 91%, 92%, 93%, 94%, 95%, 96%, 97%, 98%, or 99% identity with SEQ ID NOs: 31 and 32 respectively, as long as the FDH has the required reducing and oxidizing function.

In a biofuel cell, the FDH can be applied to an anode by adsorption, such that a very thin film of the FDH is generated on the surface of the electrode. Such adsorption can be performed, for example, by placing a protein solution including the FDH directly on the electrode, such as a pyrolytic graphite edge electrode (PGEE), letting the solution dry for a few minutes, washing off excess protein molecules, and then immersing the electrode into the electrolyte solution. This represents direct electrocatalysis because the protein is directly in contact with the electrode. Alternatively, the same procedure can be performed on a multiwalled carbon nanotube (MWCNT)-modified electrode, with the MWCNT being adsorbed onto the electrode in a similar manner as the protein. In this case, the MWCNT are sandwiched between the protein and the electrode.

As another alternative, undecaheme cytochrome c (UHC) can be adsorbed onto the electrode first, and then the FDH is adsorbed to the same surface. Since the FDH is tightly associated with UHC in nature, providing UHC to the electrode first can reduce the occurrence of FDH denaturation or loss of function during the adsorption process.

The material of the electrode is not limited, but may be, for example pyrolytic graphite edge electrode (PGEE) coated with multiwalled carbon nanotubes (MWCNT). Other alternative materials for the electrode include boron-doped diamond, carbon cloth, glassy carbon, carbon paper, and other materials known in the art. The surface of the electrode may be further derivatized, by chemical or enzymatic derivatization, to improve the binding of the protein to the electrode. Alternatively, the electrode may be pre-coated with an antibiotic, such as polymyxin.

In the fuel cell, the FDH anode is coupled with a cathode having a similar structure to the anode. The cathode may have adsorbed thereon either laccase or bilirubin oxidase (BOx). The source of the laccase and BOx is not limited, as long as it is stable and interacts with the electrode. Examples of the laccase include those derived from Trametes versicolor (Millipore Sigma catalog #38429) and Agaricus bisporus (Millipore Sigma catalog #40452). Examples of the BOx include that derived from Myrothecium verrucaria (Millipore Sigma catalog #B0390). As another alternative, the cathode may have adsorbed thereon a cytochrome oxidase (COX), such as cytochrome cbd oxidase (CydCBD, which includes the subunits CydAc and CydA′). CydCBD will be discussed in greater detail below. In addition to laccase, BOx or CydCBD, UHC may be first adsorbed onto the cathode. The cathode enzyme is adsorbed to the cathode in a similar manner as the FDH is adsorbed to the anode, described above.

As described below, a bacterial integral membrane supercomplex (also known as the “respirasome”) is made up of three proteins: formate dehydrogenase (FDH), undecaheme cytochrome c (UHC), and cytochrome oxidase (COX). Through expression of this complex in the native host and subsequent purification/characterization, it has been found that this respirasome efficiently couples formate oxidation to oxygen reduction. In this “hardwired” system, electrons derived from formate oxidation to carbon dioxide are used to reduce dioxygen, resulting in the production of water. It is noted that the term “hardwired” refers to the components of the five subunit protein complex not being in dynamic equilibrium, but rather being fixed in relative position/communication. Accordingly, electrons derived from formate oxidation by FDH are transferred through to the cytochrome oxidase via an internal ‘wire’ composed of iron-sulfur clusters and hemes without interruption, diffusion, or rearrangement. Unlike known biofuel cells which use the glucose/oxygen couple and have a lower potential difference (about 1.2 V), the disclosed biofuel cell which uses the formate/oxygen pair has a higher potential difference (about 1.7 V).

In the biofuel cell, the above-discussed electrodes are submerged in chamber including a liquid electrolyte and are electrically connected form an electrical circuit. The electrolyte may comprise a buffer, with formate and O₂ dissolved therein. Examples of a suitable buffer include Tris, sodium phosphate, and potassium phosphate, generally at a concentration of from 100 mM to 1 M. The buffer may also be a mixed system of several buffers to ensure operation between pH values of 3.5 to 10. The electrolyte may also include up to 1 M sodium chloride or up to 1 M potassium chloride as additional salts to adjust ionic strength. However, in some situations, the amount of O₂ dissolved in the electrolyte may be insufficient. In such a case, additional O₂ may be pumped or bubbled into the electrolyte, particularly for the electrode. The buffer should have a pH of about 8. Optionally, a gas-permeable membrane may separate the bioanode and biocathode chambers. However, it is preferred to include the gas-permeable membrane, in order to prevent reagents in the two chambers from mixing, but allowing H⁺ to diffuse across the membrane. This is particularly relevant in situations where the enzymatic conditions, such as pH are different in the two chambers. The structure of the gas-permeable membrane is not particularly limited. For additional information on gas-permeable membranes, see textbook “Biofuel Cells: Materials and Challenges²²², particularly pages 34-35, 72-79, 125-126, 137, and 146-151 and Li et al.²²³. Alternatively, laminar flow may be used instead of a membrane to separate the electrolyte solutions (see page 35 of citation 222).

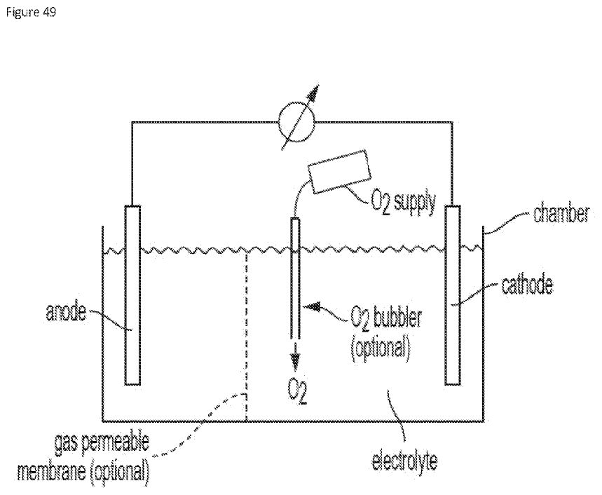

The distance between the anode and cathode is not particularly limited. FDH2 has a binding constant (Kₘ) for formate in the low micromolar range. Thus, the reaction will proceed even if the relative concentration of FDH2 and formate are both low. In the present application, kinetics experiments were done with an enzyme concentration of 1.6 nM and formate and formate in the range of 0 to 100 μM. Additionally, the density of adsorption of enzymes on the electrodes, and the sizes of the electrodes will determine the current as long as the cathode is not limiting. Additionally, the biofuel cell may include a reference electrode (RE) (not pictured) to measure the electrochemical potentials and a counter electrode (CE) (not pictured) to complete the circuit. Specifically, the RE helps to determine the precise potential difference between the CE and working electrode (WE; bioanode or biocathode). A simplified structure of the biofuel cell is illustrated in FIG. 49. As will be appreciated by those skilled in the art, industrial scale applications would require appropriate modifications, including the nature of the chambers used.

The disclosed O₂ insensitive FDH has many practical applications. First, the O₂-insensitive FDH may be used in a biofuel cell to generate electricity, as noted above. In order to generate electricity, the anode and cathode of the fuel cell are immersed in chamber including an electrolyte containing formate, and are electrically connected to form an electrical circuit. The enzymatic reaction is allowed to proceed, thereby generating electricity. The solubility of oxygen at 23° C. in water equilibrated to air is about 260 uM. As such, oxygen is readily resupplied from the air if the solution is agitated and open to the air. However, in a case where oxygen is utilized in the biocathode, the oxygen could be limiting. In this situation, direct bubbling with O₂ would prevent oxygen being limiting. Such bubbling to provide supplemental oxygen should be needed if the overall current is high relative to the volume of the electrolyte or due to increased adsorptivity of the enzyme on the electrode surface. The FDH bioanode is preferably in an environment of pH 8. The biocathode is preferably in an environment of the optimal pH of the enzyme used, and therefore may require an electrolyte and buffer appropriate to such enzyme. The concentration of the enzyme on the cathode may be adjusted as appropriate.¹⁹⁷ Additionally, a biofuel cell including the O₂-insensitive FDH may be applied to various known types of wearable electronics or implantable devices, such as a pacemaker, biosensor or contact lens.¹⁹² Use of a miniaturized fuel cell in such an implantable device would eliminate the need for a battery being included in the device. The O₂ naturally present in the body would serve to power the miniaturized biofuel cell.

Additionally, the O₂-insensitive FDH may be used in several applications other than fuel cells. For instance, the O₂-insensitive FDH can be used to generate hydrogen peroxide in an environmentally safe manner. To date, industrial manufacturing of hydrogen peroxide is performed chemically. However, the O₂-insensitive FDH can be mixed with formate and O₂ to generate hydrogen peroxide enzymatically. For example, this can be accomplished by immobilizing the FDH on a matrix, and then flowing oxygenated formate through the matrix. The FDH will then simultaneously oxidize the formate and reduce the O₂, thereby generating stoichiometric amounts of hydrogen peroxide Alternatively, hydrogen peroxide may be generated by providing the FDH in a solution, and allowing the above-noted reaction to proceed.

Another application of the O₂-insensitive FDH is a formate detection kit. Formate could be detected either in bulk or in smaller samples, such as a 96-well plate. The formate detection kit includes: (i) a reaction buffer, (ii) a formate standard as a control, (c) the FDH, and (d) a mediator dye such as phenazine ethosulfate/dichlorophenol indophenol, tetrazolium, or the like to detect formate the sample. The user would first run a control to generate a standard curve, thereby bracketing the formate concentration to be detected. Then, the user preferably would treat their sample with the buffer, the FDH and the mediator, and expose the sample to air. Instead of O₂ in air, other electron acceptors can be used, such as ferricyanide, PES/DCPIP, tetrazolium, etc. Next, the user would detect a change in color with a spectrophotometer to quantify the amount of formate. Such a formate detection kit could measure formate levels in the skin, gut, soil, or seawater for example. As for detection in the skin, this could be achieved by applying electronic skins that incorporate the FDH. This could be useful in personal nutrition, noninvasive metabolite profiling, including in exercise metabolomics, identification of biomarkers, and in specific diagnosis of certain skin disorders. As to the detection of formate in the gut, this could be applied by providing a non-invasive capsule which would allow recording of formic acid levels detected by the FDH using microelectronics. Additionally, the O₂-insensitive FDH also can be applied to a device which serves as a safety indicator in the manufacture of methanol or chemical with reactive methyl groups, because the formate metabolite would rise with exposure.

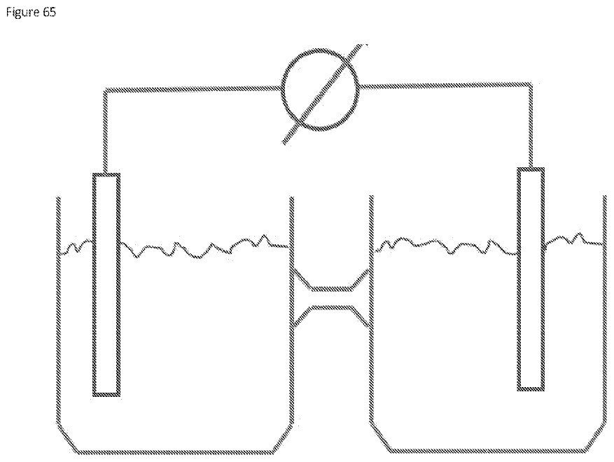

Another embodiment is a fuel cell which allows for simultaneous generation of electricity and H₂O₂. In this embodiment, FDH2 is adsorbed on both an anaerobic anode (dehydrogenase activity) and an aerobic cathode (formate oxidase activity). This is illustrated in FIG. 65. This is similar to other disclosed embodiments, exception that it is necessary to limit additional oxygen coming into the anode, by closing the half cell vessel, for example with a lid. Like known air-sensitive FDH fuel cells, an anaerobic anode and aerobic cathode are present and oxygen is excluded from one half cell while providing it to the other. This allows for FDH2 to form H₂O₂ without inhibition by O₂ or H₂O₂ itself. As shown in FIG. 65, half cells are in relative isolation and only connected by a membrane, salt bridge or frit.

Additionally, the O₂-insensitive FDH can be applied to carbon capture strategies by running the DvH-FDH2 catalyzed reaction in reverse. In the above-discussed biofuel cell, a forward reaction proceeds (formate oxidation, which produces CO₂ as product and 2 electrons). The electrons to flow through the bioanode and through the electric circuit reach the biocathode. In other words, electrons from formate oxidation flow onto the anode through electrical wires that connect the bioanode to the biocathode and onto an oxidase, while the aqueous connection between the two parts of the cell (or salt bridge) allows for charge balance (migration of positive charge in the form of protons or cation) to complete the circuit. The enzyme on the biocathode (for example, BOx, laccase, or a CydCBD enzyme) uses the two electrons to reduce O₂ to H₂O. This reaction requires 4 electrons and 4 protons 2O₂+4H⁺+4e⁻→2H₂O; or ½O₂+2H⁺+2e⁻→2H₂O). However, the reaction can be reversed to consume CO₂ from air (or other sources such as burning oil, gas, biomass, or directly from seawater) as substrate and generate formate, which is a microbial feedstock. Formate as a feedstock is metabolically equivalent to H₂, thus it can be considered a stable storage form of H₂ and CO₂. Although several FDH enzymes from different bacteria have been investigated for their ability to catalyze the reverse reaction, none of these can perform the reverse reaction in air, due to their O₂-sensitivity.

However, since the disclosed FDH is O₂-insensitive, it can be applied to the capture of CO₂ without inactivating the enzyme in air. Nearly all sources of CO₂ are contaminated with other gases, including carbon monoxide and O₂. However, the O₂ insensitive FDH is unaffected by carbon monoxide and O₂ and, therefore, can be used for carbon capture and related green applications.

Whereas the forward reaction releases electrons, the reverse reaction requires input of electrons. Although reactions using some chemicals such as viologens (the same molecules that in the context of a polymer gel confer protection from O₂) have been attempted, these will cease to work in air. This is because they will readily oxidize before being able to donate the electrons to the protein.

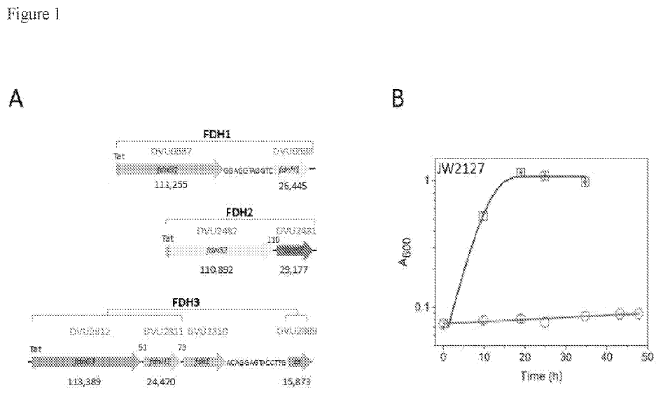

This problem can be avoided by using an electrode to inject electrons into the enzyme so that it can reduce CO₂ and produce formate. This is illustrated for example in FIG. 1A of Sokol et al.¹⁹⁸ However, although the system of Sokol can harvest electrons from sunlight and donate them to the FDH, it cannot run in air.

It should also be noted that in the carbon capture application, the electrochemical cell configuration is reversed. That is, the O₂-insensitive FDH is immobilized on the biocathode, rather than the bioanode, so that it can obtain electrons from the bioanode. Air, containing CO₂, is bubbled or pumped into the catholyte. Alternatively, sodium carbonate or sodium bicarbonate, both of which serve as a CO₂ source when dissolved in water, could be used. Note that the CO₂ reduction reaction must be performed at pH 6 or below so that enough CO₂ remains in solution. In this case, the bioanode enzyme could be photosystem II¹⁹⁸, photosystem I, or any other system that can serve as electron acceptors.

As another alternative to electrode delivery, cadmium sulfide (CdS) or cadmium selenide (CdSe) quantum dots (QD) can be used in a manner similar to that disclosed in Edwards et al.¹⁹¹ CdS or CdSe can be used to serve as an electron source when light is shined on the QD. Additionally, the QD can be derivatized (or modified) in numerous ways to help the enzyme favorably interact with it. Additionally, hydrogen peroxide is generated in this method.

Next, details are provided with respect to operation and structure of the O₂-insensitive FDH. As noted above, the DvH-FDH2 has a first subunit represented by SEQ ID NO: 31 and a second subunit represented by SEQ ID NO: 32. However, the FDH is not limited to this. Rather, an FDH may be utilized which has one or more additions, deletions, or substitutions relative to SEQ ID NOs: 31 and 32. For instance, the first and second FDH subunits may each have 90%, 91%, 92%, 93%, 94%, 95%, 96%, 97%, 98%, or 99% identity with SEQ ID NOs: 31 and 32 respectively, as long as the FDH has the required reducing function.

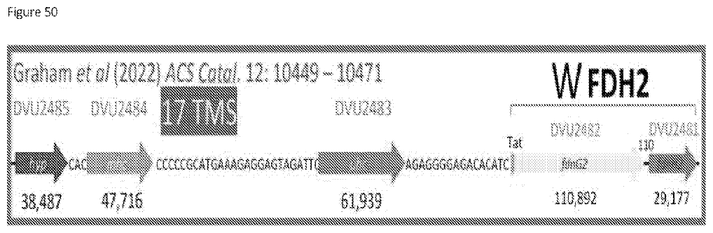

The FDH can be produced on its own, or as part of a five-gene operon represented by SEQ ID NO: 36. This operon described in 2004 as part of the genome sequence of DvH⁵⁷. Although DvH-FDH2 was isolated and partially characterized in 2011⁶⁸, the characterization was performed in the complete absence of O₂. Therefore, it was not previously known that the FDH is O₂-insensitive. The five-gene operon includes the following genes: (i) DVU2481, encoding the small subunit fdnH2 of FDH2, (ii) DVU2482, encoding large subunit fdnG2 of FDH2, (iii) DVU2483, encoding an 11-heme undecaheme cytochrome c (uhc), (iv) DVU2484, encoding monoheme cytochrome c (mhc), also known as CydAc, the catalytic subunit of cytochrome cbd oxidase (CydCBD), and (v) DVU2485, encoding a formerly hypothetical protein (hyp), now characterized subunit of CydCBD and referred to as CydA′. The structure of the operon is illustrated in FIG. 50.

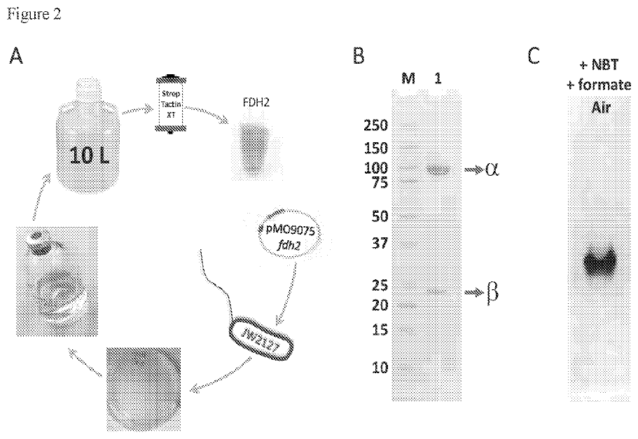

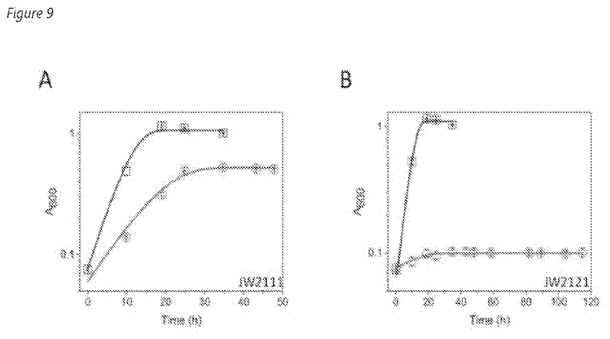

Robust Expression Platform for Facile Production of Highly Pure O₂-Insensitive Metallo-FDHs. There are three distinct fdh loci in the DvH genome⁶⁹ (FIG. 1A). Only FDH1 encoded by the first locus is essential for growth when sulfate and formate serve as electron acceptor and electron donor, respectively.⁶² The cellular functions of FDH2 and FDH3 are not well defined. Oliveira et al²³ expressed FDH1 in a Δfdh1 deletion strain. The construction of a markerless FDH-free strain could be beneficial on three fronts: (a) Facilitate biochemical investigations of a native or foreign FDH without potential interference from host counterparts, (b) Benchmark whole cell biocatalysts, and (c) Uncover how synergy between enzyme catalysis and bioenergetics modulates organismal dynamics. To that end, a DvH strain (JW2127; see Methods; Tables 6 and 7) was generated that is devoid of all three fdh loci. Although JW2127 is unable to grow on formate-acetate-sulfate, it maintains wild-type-like growth profile on lactate-sulfate medium (FIG. 1i). Deletion strains were constructed harboring all possible combinations of fdh genes for functional analyses, including JW2111 (Δfdh3) and JW2121 (Δfdh1 and Δfdh3; see Tables 6 and 7). The latter two served as controls in this study (FIG. 9). Subsequently, JW2127 was used for the homologous expression of FDH2. Introduction of a Strep-tag II at the C-terminus of the large subunit facilitated one-step affinity purification. An overview of the protein purification method is shown in FIG. 2A. Whereas Oliveira et al.²³ used DvH cells derived from 300 L fermentation to purify FDH1, the present workflow was streamlined to produce 1.8 mg of highly pure heterodimeric FDH2 from a gram of wet cell paste (FIG. 2A,B). Thus, 10 L culture (biomass yield of ˜8 g) generates sufficient protein to tackle a broad range of experiments. Since most laboratories do not have access to large-scale anaerobic fermentation, this method also offers a facile path to metalloprotein production. Importantly, there is a fundamental difference between prevailing strategies for metallo-FDH isolation and what has herein been advanced. The purification workflow (FIG. 2A) and downstream handling steps (including storage) occur in air without involving nitrate, azide, thiols, or formate at any stage of the process.

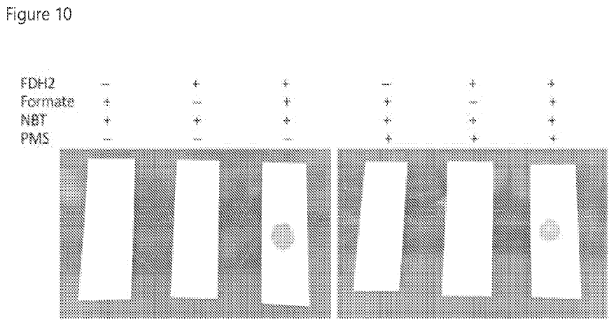

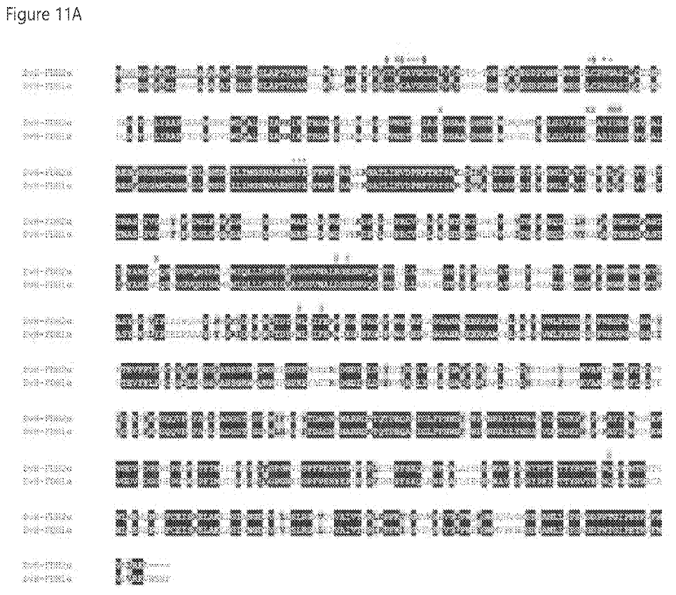

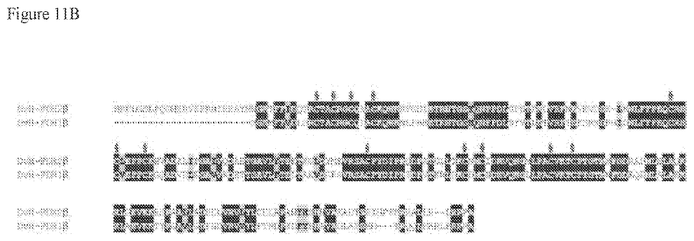

Aerobic In-Gel Catalysis of Recombinant DvH-FDH2. Literature precedents exist for anaerobic activity staining of FDHs in native polyacrylamide gels using 2,3,5-triphenyltetrazolium chloride^{68,71} or phenazine methosulfate (PMS)/nitroblue tetrazolium chloride (NBT).⁷²⁻⁷⁴ However, this has not been achieved for any FDH in air. Because O₂-insensitive group 5 [NiFe]-hydrogenases have been zymographically visualized using redox dyes,⁷⁵ a similar approach was considered with DvH-FDH2. When native polyacrylamide gel strips containing recombinant DvH-FDH2 were incubated aerobically with NBT and formate, a single dark blue colored band appeared within two minutes (FIG. 2C). In the absence of formate, this band was not observed (FIG. 10). The same pattern was recapitulated in the spot assay where the blue color developed within 15 s (FIG. 10). These observations demonstrate that electrons released from enzymatic aerobic formate oxidation are readily transferred to an artificial electron acceptor with high reduction potential (E_{m,7}=+50 mV^{32,75,76}), resulting in the generation of insoluble reduced NBT-formazan precipitates. These observations further demonstrate that both nitrate-assisted purification of FDH and/or reductive activation with high concentration of thiols are not essential for maintaining redox activity under anaerobic or atmospheric conditions.

[4Fe-4S] Metalloclusters, Tungstopterin, and Selenocysteine Remain Unaffected by O₂ During Catalytic Turnover. Metal specificity profiles of SRB FDHs remain incompletely described⁷⁷. Moreover, the nature of redox centers in DvH-FDH2 has not been established.⁶⁸ Because DvH-FDH1 and DvH-FDH2 exhibit 61% protein sequence identity (large catalytic subunit) and share all the metal coordination sites within the two subunits (FIGS. 11A and 11B), it was believed that a similar complement of redox centers must exist in both systems. Since the DvH biomass was derived from a medium containing Mo (1.24 μM) and W (0.15 μM), a metal ratio of 1Mo/W: 16Fe:1Se was predicted. Consistent with this, inductively coupled plasma mass spectrometry (ICP-MS) revealed that for every mole of ¹⁸²W present, another 17±1 moles of ⁵⁶Fe and 0.7±0.1 moles of ⁷⁸Se were also found (Table 1). Despite the nine-fold excess of molybdate (excluding contributions from yeast extract) in the growth culture, ⁹⁵Mo was not detected in FDH2 samples. These results underscore definitive tungsten selectivity of DvH-FDH2, distinguishing it from Mo-specific^{17,68} DvH-FDH3 and the promiscuous DvH-FDH1, which is capable of incorporating both Mo and W.^{23,68}

TABLE 1
ICP-MS quantification of metal cofactors in DvH-FDH2

| | | Replicate 1 | [Metal] | Replicate 2 | [Metal] | Replicate 3 | [Metal] | |
|---|---|---|---|---|---|---|---|---|
| Metal | Predicted | ng mL⁻¹ | μM | ng mL⁻¹ | μM | ng mL⁻¹ | μM* | Observed |
| Fe | 16 | 82 ± 2.8 | 1.47 ± 0.05 | 42.8 | 0.76 | 35 ± 0.1 | 0.625 | 17 ± 1 |
| Mo | 1 | ND | ND | ND | ND | ND | ND | ND |
| Se | 1 | 4.3 ± 0.1 | 0.055 ± 0.002 | 2.2 | 0.028 | 2.5 ± 0.1 | 0.032 | 0.7 ± 0.1 |
| W | 1 | 15.09 ± 0.02 | 0.083 ± 0.003 | 8 | 0.044 | 7.5 ± 0.1 | 0.41 | 1 ± 0.1 |

Errors are standard deviations from triplicate measurements using protein samples derived from three independent preparations;
ND, not detected
*N = 2,
SEM < 0.001

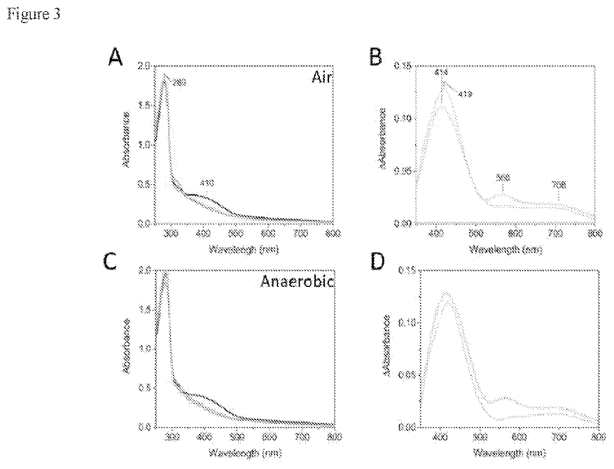

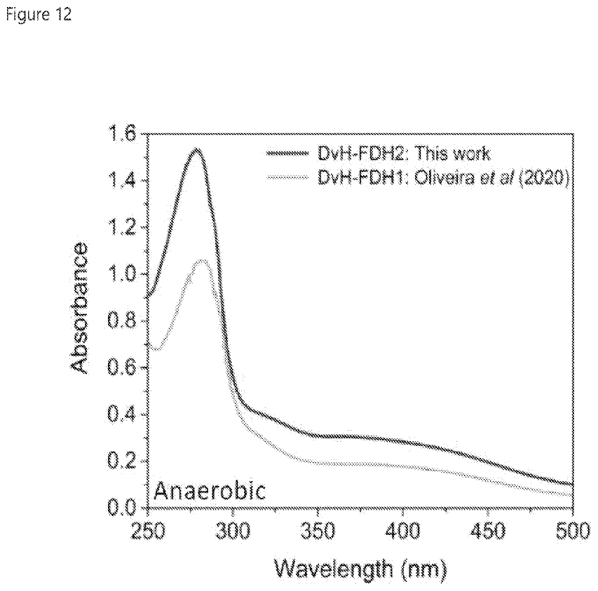

Electronic and Electron Paramagnetic Resonance (EPR) Spectral Signatures of DvH-FDH2 are Virtually Invariant in Air. The bulk of metallo-FDH electronic spectra in the primary literature have been measured under anaerobic conditions to avoid inactivation my molecular O₂.^{16,19,23,32,78} Although aerobic spectra exist for an O₂-tolerant Mo-Cys-FDH stabilized by 10 mM nitrate,⁴⁰ their utility remains unclear, for the addition of formate did not afford a characteristic spectral change. Similarly, formate-reduced spectra in air are not available for metallo-FDHs characterized from either methanotrophs³⁶ or methylotrophs.⁷⁹ Here, the first functional validation of a W-Sec-FDH in air via electronic spectroscopy is shown. Aerobically purified DvH-FDH2 is brown in color and shows a broad S→Fe³⁺ charge transfer transition at 412 nm (FIG. 3A, blue trace), which is characteristic of [4Fe-4S]²⁺ clusters.⁸⁰ Addition of formate leads to a substantial loss of this signal, indicating reduction to the [4Fe-4S]⁺ state (FIG. 3A, green trace). Reduction with dithionite yields a similar result (FIG. 3A, orange trace). Employing anaerobic conditions makes no difference to the outcome (FIG. 3C). The virtually identical line shape and amplitude of the difference spectra (FIG. 3B, 3D) illustrate that formate completely reduces the majority of catalytically competent FDH2 in solution. Ligand→W charge transfer transitions are also visible at 568 and 708 nm. As dithionite would be expected to reduce both functional and non-functional metal centers, it was concluded that >94% of DvH-FDH2 is functionally fit. The source DvH-FDH1 spectrum (Figure S4, orange trace, of Oliveira et al (2020)) was obtained and compared with an as-isolated DvH-FDH2 counterpart acquired under anaerobic conditions (FIG. 12). The A₄₀₀/A₂₈₀ ratio—an indicator of the extent of cluster loading⁸¹—estimated from these spectra are 0.18 (DvH-FDH2) and 0.17 (DvH-FDH1), affirming that the two orthologs exhibit comparable protein purity and cofactor integrity.

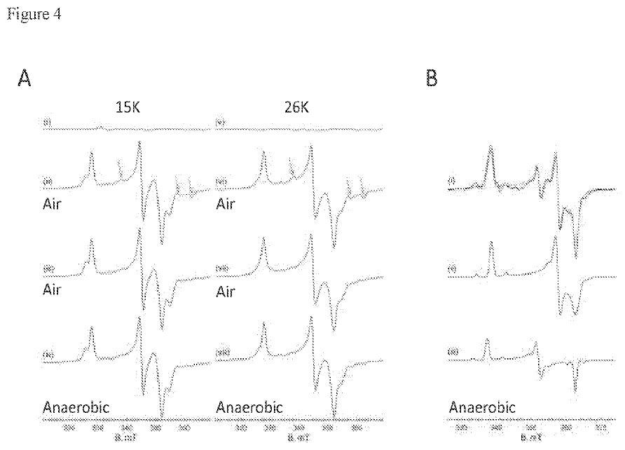

To evaluate the predictions made via UV/visible spectroscopy, electron paramagnetic resonance (EPR) measurements were taken. The oxidized enzyme is EPR-silent and specifically devoid of signals that might be attributed to oxidized [3Fe-4S] clusters [FIG. 4A, panels (i) and (v)]. On the other hand, under a variety of reducing conditions, DvH-FDH2 exhibits signals that are characteristic of reduced [4Fe-4S] clusters. At 15 K, a minimum of two distinct EPR signals were observed (FIG. 4A, panels (ii)-(iv)), one of which is significantly broadened at 26 K (FIG. 4A, panels (vi)-(viii)). By 40 K, both signals have disappeared (data not shown), a behavior that is typical of fast-relaxing [4Fe-4S] clusters.

The relative intensities of the two signals at a ratio of 1:0.75 are essentially independent of whether formate or dithionite was used as a reductant, either aerobically or anaerobically. The integrated intensity amounts to 4.1±0.2 spins per protomer, indicating that both dithionite and formate result in full reduction of all four [4Fe-4S] clusters of the protein. This conclusion is consistent with the observed UV/visible absorption changes (FIG. 3), indicating complete reduction of the enzyme under these conditions, with the implication that there are two pairs of [4Fe-4S] clusters with similar g-values.

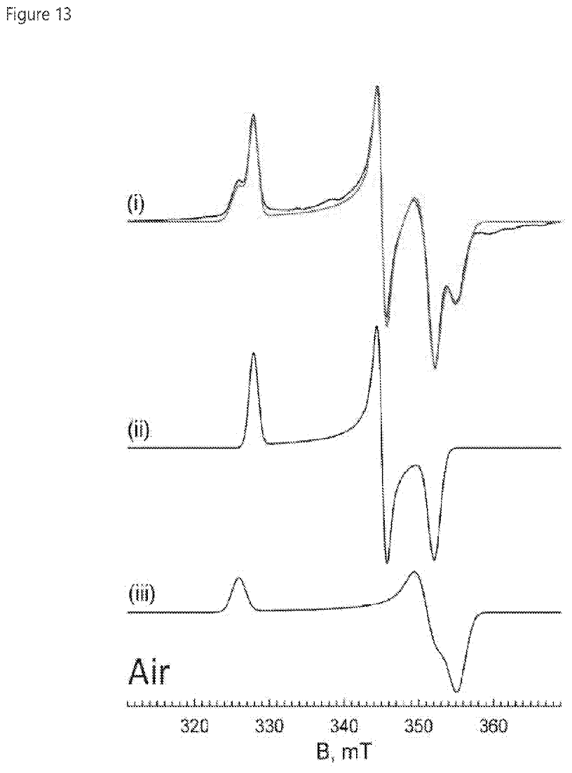

The simulated spectrum for the formate-reduced DvH-FDH2 prepared under aerobic conditions and collected at 15K from FIG. 4(iii) is shown in FIG. 13, and the simulation parameters given in Table 2. The g-values obtained are again consistent with iron-sulfur clusters and the relative contribution of each cluster No indication of additional signals is evident at higher microwave power, and no signals are observed above g=2.1 that might suggest S>½ states. The absence of additional signals in the four-cluster FDH2 seen here is reminiscent of D. gigas W-FDH1 results,⁸² where the two observed signals represent pairs of Fe/S clusters with similar g-values.

TABLE 2
EPR simulation parameters for
W^{V} and reduced Fe/S centers of Fdh2

| | | | | | Tungsten coupling | | | |
|---|---|---|---|---|---|---|---|---|
| | g tensors | | | | constants^{a} | | | Relative |
| Center | g₁ | g₂ | g₃ | gₐᵥₑ | A₁ | A₂ | A₃ | distribution |
| W^{V}1^{b} | 1.982 | 1.876 | 1.849 | 1.902 | 232 | 119 | 151 | 10 |
| W^{V}2^{b} | 1.988 | 1.904 | 1.849 | 1.914 | 233^{c} | 131 | 125 | 0.54 |
| Fe/S1^{d} | 2.045 | 1.943 | 1.904 | 1.964 | | | | 1.0 |
| Fe/S2^{d} | 2.058 | 1.910 | 1.888 | 1.952 | | | | 0.75 |

^{a}In MHZ; in the absence of multi-frequency data, coupling constants are approximate
^{b}Spectrum collected at 108K
^{c}Fixed during simulations
^{d}Spectrum collected at 15K

When 150 μM enzyme is incubated with dithionite under anaerobic conditions for an extended amount of time (˜12 hours or more) and the spectrum is collected at 108K, an additional pair of signals are obtained (FIG. 4B(i)); there is no evidence of the Fe/S signals described in FIG. 4, panel A and 13 at this temperature. The new signals persist from 15K all the way to 108K without considerable line broadening, consistent with their arising from slowly relaxing W(V) species. The simulation parameters are presented in Table 2 and include the well-resolved tungsten I=½ hyperfine splittings originating from the 14.3% natural abundance ¹⁸³W isotope. The presence of the I=½ hyperfine splitting is further evidence that these signals arise from the tungsten center rather than additional Fe/S clusters. FIG. 4B(ii) and (iii) show the component spectra scaled to their contribution to the composite simulation in FIG. 4B(i). The simulations indicate that the two species are in an approximate ratio of 1:0.54 and the principal g-values (g₁₋₃=1.982, 1.876, 1.849 and 1.988, 1.904, 1.849, respectively), in good agreement with those seen from other W-containing enzymes. Somewhat surprisingly, the large anisotropy of the W(V) g-values more closely resembles the “low potential” signal for the Pyrococcus furiosus aldehyde ferredoxin oxidoreductase (AOR), which is a member of a different family of tungsten-containing enzyme than the FDHs.⁸³ The presence of multiple W(V) signals in a single sample has been seen with a number of W-containing enzymes and may be due to the presence of inactive species in addition to the catalytically competent one, which is a rather common feature of W-containing enzymes.⁶⁴

Full Progress Curves Reveal High Catalytic Efficiency Under Atmospheric Conditions and Lack of Enzyme Inactivation or Product Inhibition. Solution enzyme kinetics investigations of metallo-FDHs have not directly probed formate depletion or CO₂ production. Instead, low-potential artificial electron acceptors, most commonly benzyl viologen (BV; E_{m,7}=−360 mV³²) and methyl viologen (MV; E_{m,7}=−446 mV³²) for the forward and reverse reactions, respectively, have been routinely used as surrogates to report on catalytic robustness. Although cautions have been raised against trusting kinetic parameters derived from the use of these “inefficient and slow redox mediators”^{27,85}, they continue to be favored. Mo-Cys-FDHs offer an alternative by making it possible to track NAD⁺ reduction or NADH oxidation.^{32,40} Unfortunately, this strategy cannot be extended to all metallo-FDHs and it is prone to yield false-positive results when interrogating aerobic CO₂ reduction with aerotolerant FDHs.²⁶ To further complicate matters, FDHs from sulfate-reducing bacteria (SRB) are in a class of their own (Table 3). Moreover, there are no reports on metallo-FDH enzymology that has disclosed a complete set of raw absorbance versus time data used to extract kinetic parameters. Table 3: Literature stead-state kinetics parameters of SRB-FDHs

| | | | T | [BV] | [Enzyme] | | Kₘ | |
|---|---|---|---|---|---|---|---|---|
| System | Organism | pH | (° C.) | (mM) | (nM) | k_{cat} (s⁻¹) | (μM) | Reference |
| W- | Da | 8.0 | 37 | 7.5 | 35 | 241 | 10 | Mota |
| FDH1^{WT} | | | | | | | | (2011); |
| | | | | | | | | Mota et al |
| | | | | | | | | (2011) |
| W- | Dg | 8.0 | 37 | 7.5 | 35 | 174 | 51 | Mota |
| FDH1^{WT} | | | | | | | | (2011); |
| | | | | | | | | Mota et al |
| | | | | | | | | (2011) |
| W- | DvH | 7.6 | RT | 2 | 0.0124 | 3684 | 1 | Da Silva |
| FDH1^{WT} | | | | | | | | et al |
| | | | | | | | | (2011) |
| W- | DvH | 7.6 | RT | 2 | 1.4 | 1100 | NR | Miller et |
| FDH1^{REC} | | | | | | | | al (2018) |
| W- | DvH | 7.6 | RT | 2. | 1.4 | 940 | NR | Szczesny |
| FDH1^{REC} | | | | | | | | et al |
| | | | | | | | | (2019) |
| W- | DvH | 7.6 | RT | 2 | 1.4 | 1104 ± 62 | NR | Oliveira et |
| FDH1^{WT} | | | | | | | | al (2020) |
| W- | DvH | 7.6 | RT | 2 | 1.4 | 1310 ± 50 | 16.9 ± 2.8 | Oliveira et |
| FDH1^{REC} | | | | | | | | al (2020) |
| W- | DvH | 7.6 | RT | 2 | 1.4 | 1144 | NR | Alvarez- |
| FDH1^{REC} | | | | | | | | Malmagro |
| | | | | | | | | et al |
| | | | | | | | | (2021) |
| ??- | DvH | 7.6 | RT | 2 | 0.48 | 81 | 4 | Da Silva |
| FDH2^{WT} | | | | | | | | et al |
| | | | | | | | | (2011) |
| Mo- | Dd | 7.6 | 37 | 7.5 | 35 | 357 ± 18 | 65 ± 8 | Rivas et al |
| FDH3^{WT} | | | | | | | | (2007) |
| Mo- | Dd | 8.0 | 37 | 7.5 | 35 | 347 | 64 | Mota |
| FDH3^{WT} | | | | | | | | (2011); |
| | | | | | | | | Mota et al |
| | | | | | | | | (2011) |
| Mo- | DvH | 7.6 | RT | 2 | 0.25 | 262 | 8 | Da Silva |
| FDH3^{WT} | | | | | | | | et al |
| | | | | | | | | (2011) |
| Mo- | Dd | 8.0 | 22 | 5 | 1 | 543 | 57 | Maia et al |
| FDH3^{WT} | | | | | | | | (2016) |

SRB, sulfate-reducing bacteria; WT, wild-type natively-purified protein; REC, recombinant; W-, tungsten-containing; Mo-, molybdenum-containing; ??, metal status unknown; NR, not reported; likely to be similar to the value reported by Oliveira et al (2020); RT, room temperature; BV, benzyl viologen; DvH, Desulfovibrio vulgaris Hildenborough; Dd, D. desulfuricans; Dg, D. gigas; Da, D. alaskensis. Additional experimental details shared by Drs. Luisa Maia and Inês Pereira have been included here for the sake of completeness. The FDH probed in the present application is “??- FDH2^{WT}”.

To resolve these uncertainties, solution enzyme kinetics approaches were explored capable of yielding results with functional information content. First, a qualitative assessment of electron acceptor specificity was performed. See video at pubs.acs.org/doi/suppl/10.1021/acscatal.2c00316/suppl_file/cs2c00316_si_002.mp4 or ndownloader.figstatic.com/files/36617584.

The video visualizes aerobic formate oxidation. Video playback is in real time and the time stamps described below is in hours:minutes:seconds format. Experimental conditions are identical to those specified in Figure S38. PES/DCPIP reduction is shown first. FDH2 is added at 00.00:09. The reaction goes to completion within the next 11 s (00:00:20). Ferricyanide (FC) reduction is initiated at 00:00:24. Reaction completes around 00:02:05. Aerobic BV reduction setup is ready at 00:02:11. Observe that the cuvette is exposed to air and stirring is enabled. BV reduction is triggered at 00:02:14. Blue schlieren is transiently visible as streaks but quickly disappears. Stirring is stopped 00:02:30 to prevent further oxygenation of the reaction mixture. Blue color begins to develop at 00:03:40 and dominates the solution by 00:04:00. At 00:04:05 the cuvette is stoppered to slow down O₂ entry into the cuvette. At 00.04:26 stirring commences, resulting in the complete mixing of the solution. The BV reduction measurements conducted in air should not be confused with what had been reported by Maia et al (2016). Authors of the latter work exposed the reaction vessel to air only after anaerobic BV reduction was completed. Consequently, Maia et al had no way to assess whether the reaction continued to progress in the presence of O₂ or ceased completely. The BV reaction mix was under atmospheric O₂ until the reduction was complete. It was never made anaerobic by N2/Ar sparging. Nor was any other method used to make the solution anaerobic. This visual demonstration serves as incontrovertible direct proof that electrons derived from formate oxidation simultaneously end up on two different electron acceptors.

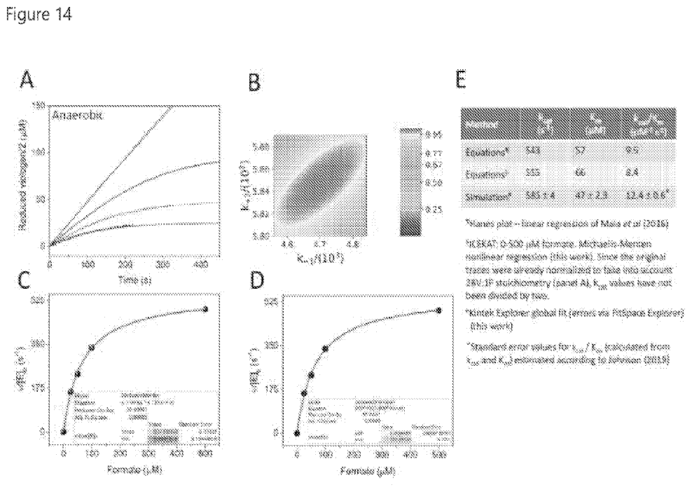

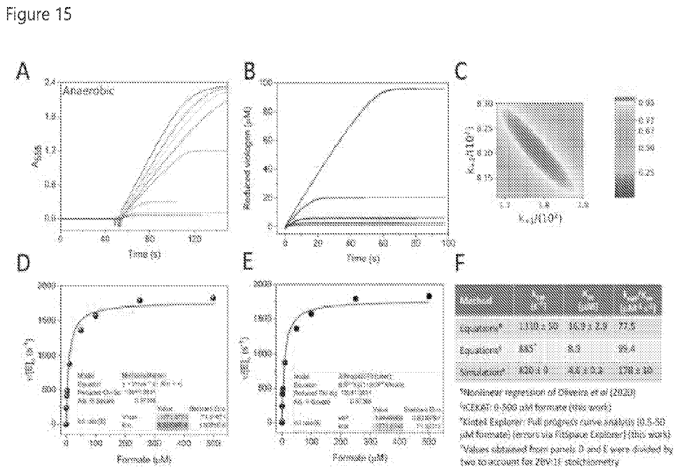

Two chemically distinct artificial electron acceptors were then settled on, one each from the low- (BV) and high-potential [phenazineethosulfate (PES)]E_{m,7}=+65 mV⁸⁶ categories. The advantage of the latter is that it can be readily coupled with dichlorophenolindophenol (DCPIP) (E_{m,7}=+217 mV⁸⁶) for facile acquisition of kinetics data both in air and under argon. Second, conditions were identified under which full-progress curves could be measured. Such an approach is only possible for stable enzymes that catalyze a single-substrate irreversible reaction in the absence of enzyme inactivation or product inhibition.⁸⁷⁻⁹⁰ Third, simultaneously analysis was performed of several full-progress curves using dynamic simulation-based global fitting⁸⁹ to extract k_{cat} and k_{cat}/Kₘ. This strategy overcomes the limitations of classical steady-state analysis, such as the use of only the first few seconds of data, unreliable initial-velocity values, and overparameterization.⁸⁴ To benchmark these models (Schemes 1 and S1), source BV enzyme kinetics data was obtained that formed the basis of Figures S3 and 1C of Maia et al.¹⁰ (D. desulfuricans FDH3) and Oliveira et al.²³ (DvHFDH1), respectively. Global fitting of the steady-state progress curves from Maia allowed for recapitulation of the published values (FIG. 14). Although Oliveira et al.²³ only reported results from the initial velocity data, five full-progress curves were extracted from this source data and de novo analysis was performed (FIG. 15(A)-(C)). In addition to finding k_{cat} values in the reported range, this method redefines the Kₘ of DvH-FDH1 to be 4.6±0.3 μM rather than 17 μM (FIG. 15 (D)-(F). These observations illustrate that the catalytic models are poised to extract reliable kinetic parameters from DvH-FDH2 progress curves.

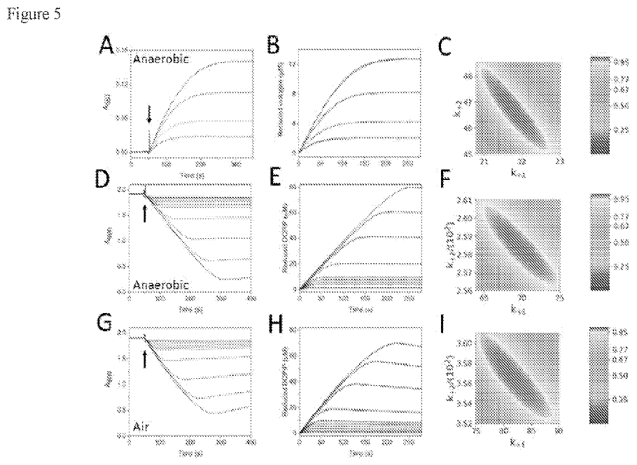

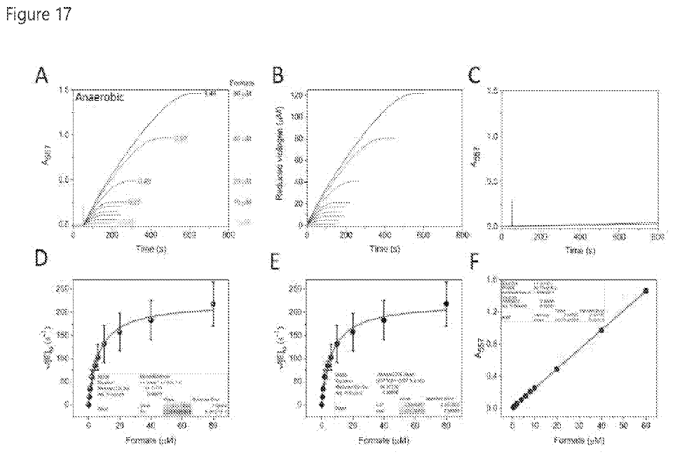

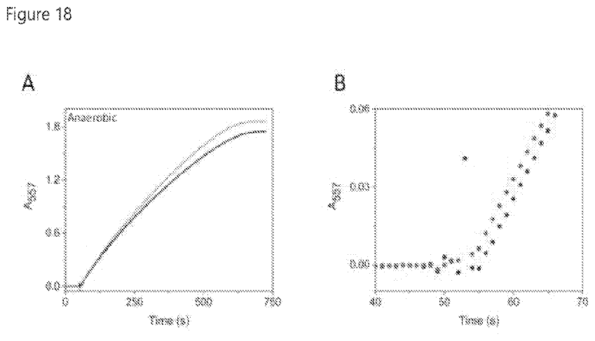

Because the original characterization of native DvH-FDH2—by the same laboratory that has reported extensively on DvHFDH1—was done using 2 mM BV (see Table 3)⁶⁸, reproduction of the published results with aerobically purified recombinant DvH-FDH2. However, the enzyme was added to the reaction mix without any preactivation using thiols or formate. Although DvH-FDH2 displays redox activity in air (FIG. 2C), nonenzymatic reaction of reduced BV⁺ with O₂ limited to strictly anaerobic conditions with this electron acceptor. Since measurements were not made inside an anaerobic chamber, anoxic conditions were ensured by adding 1 unit mL⁻¹ of glucose oxidase (GO). Catalase was also included to eliminate H₂O₂ that may arise from GO activity. Global fitting of full-progress curves (1, 2,4, and 6 μM formate) yielded values comparable to those reported by Silva et al.⁶⁸ (FIG. 5A-C and Table 5). Furthermore, the maximal absorbance values reached in the progress curves revealed that two molecules of BV⁺ are generated for every formate molecule oxidized by DvH-FDH2. Despite elimination of the reductive activation step, the enzymatic parameters derived from the standard steady-state kinetics analysis were virtually identical to the published values (Table 4), suggesting that the preactivation step introduced by Silva et al. had no effect on the outcome. On the one hand, DvH-FDH2 exhibits catalytic parameters that are roughly an order of magnitude smaller than their DvH-FDH1 counterparts (Table 4 and FIG. 15(F)). On the other hand, the values are in the same range as those derived for fully activated DvH-FDH1 (compare Figure S5A and Table 1 of Oliveira et al.²³). A closer inspection of product stoichiometry at high formate concentrations suggested that BV concentration could be limiting (Figure S8). Therefore, the experiments were repeated at 20 mM BV. This restored 2BV⁺:1 formate stoichiometry across the board but catalytic parameters did not change appreciably (FIG. 17, Table 4). It has also been confirmed that addition of GO and catalase do not interfere with the results of activity measurements (FIG. 18).

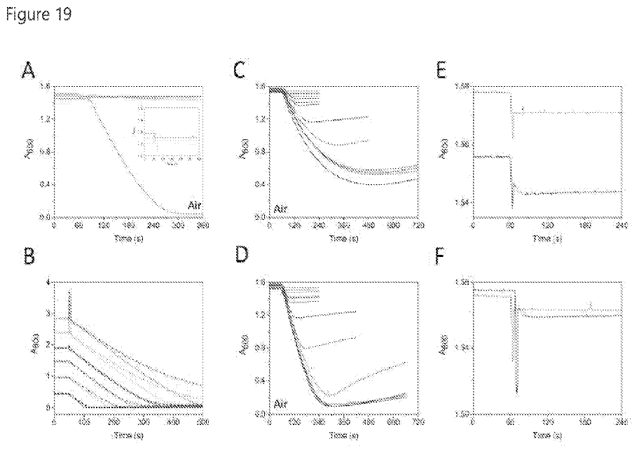

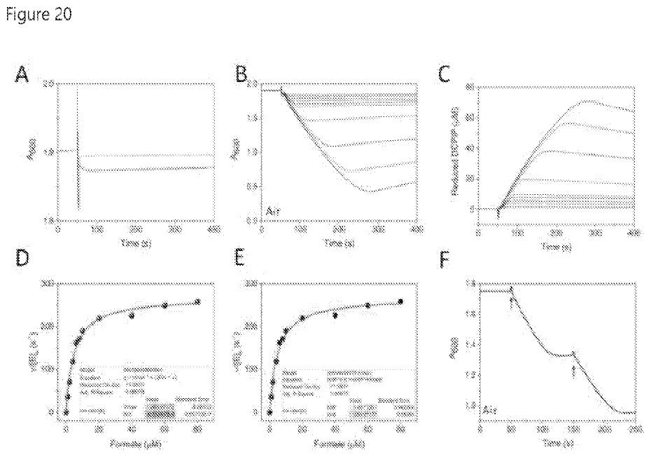

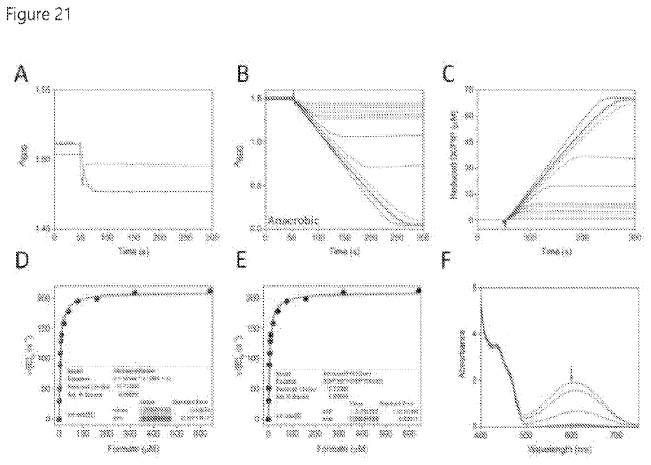

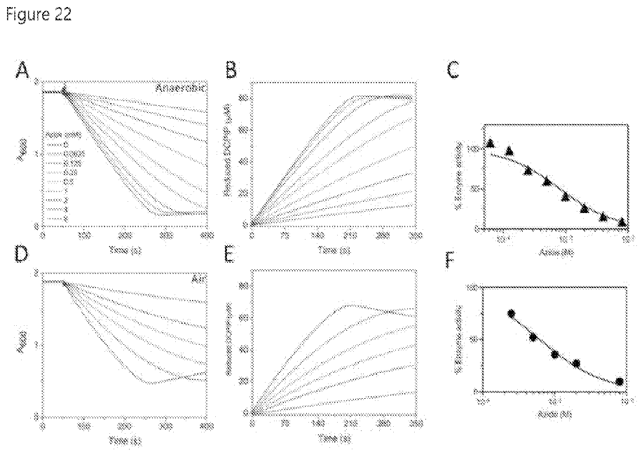

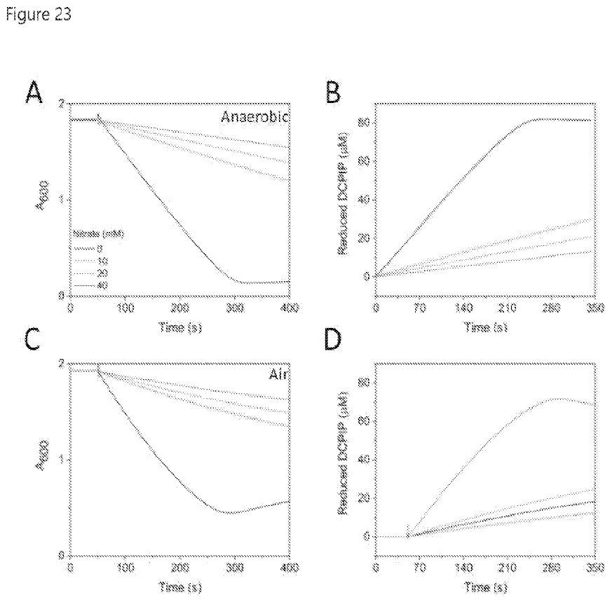

Collectively, the observations above suggest that BV is not a good electron acceptor for DvH-FDH2. To test this hypothesis, activity assays were independently pursued with the PES/DCPIP pair. As has been observed for other dehydrogenases⁹¹, PES served as an efficient electron acceptor for DvH-FDH2 (FIG. 19(A)). By varying the concentrations of DCPIP and PES, it was possible to identify optimal conditions that would support activity measurements both in air (FIGS. 19(B)-(F) and 20) and under argon (FIG. 21). At DCPIP concentrations below 100 μM, global fitting of anaerobic full-progress curves (Scheme 1 and FIG. 5D-F; 1, 2, 4, 6, 8, 10, 20, 40, 60, and 80 μM formate) resulted in roughly a 5-fold higher TN than what was obtained with 2 mM BV (Table 5). Kₘ values did not show a significant difference, however. The same pattern was reproduced when the measurements were made in air (FIG. 5G-I). A key difference between the two conditions is that the slow reoxidation (k₊ₛ=3±0.5×10⁻⁶ M⁻¹ s⁻¹; see the last equation in Scheme 1) of reduced DCPIP by O₂ caused the post-reaction region to slope slightly upward (FIGS. 5(G), 19(D), and 20(B)). However, this should not be confused with alterations to the shape of progress curves stemming from product inhibition⁸⁹, which was not observed when DCPIP (FIG. 5(G)) or BV (FIG. 5(A)) served as electron acceptors. In fact, a product stoichiometry of one reduced DCPIP for every formate oxidized was reproducibly found in the measurements (Table 5). Since addition of a fresh substrate at the end of a progress curve cleanly reproduced the original trace (FIG. 20(F)), it can be further ascertained that the enzyme was stable and fully active during catalysis in air. Taken together, PES-/DCPIP-dependent catalytic parameters obtained from global fits are in excellent agreement with those from the initial velocity calculations (Table 4). Also, the TN with PES/DCPIP remains virtually the same under anaerobic and aerobic conditions. The catalytic efficiency of DvH-FDH2 in air is also in the range of 7×107 M¹ s⁻¹ (Table 5), which is comparable to that reported for DvH-FDH²³ (˜8×107 M⁻¹ s⁻¹) when BV serves as the electron acceptor under anaerobic conditions. Moreover, the PES/DCPIP-based TN and k_{cat}/Kₘ for formate oxidation are roughly an order of magnitude and 500-fold higher, respectively, than what has been described for the aerotolerant Mo-Cys-FDH from Rhodobacter capsulatus using the natural (NAD⁺) electron acceptor.⁴⁰ Finally, the inhibition profiles of DvH-FDH2 in the presence of azide or nitrate are not significantly impacted by O₂ (FIGS. 22 and 23). Whereas azide blocks the enzyme with an IC₅₀ of about 0.8 mM, nitrate is far less effective.

See Scheme 1 below:

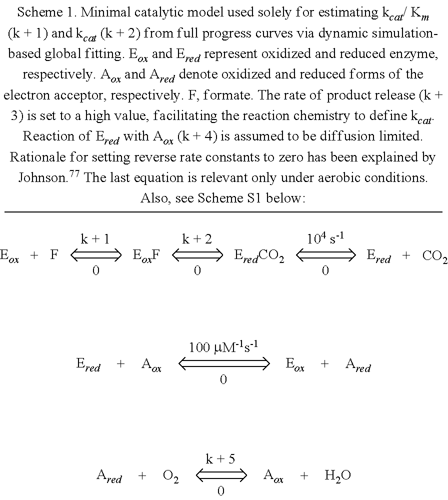

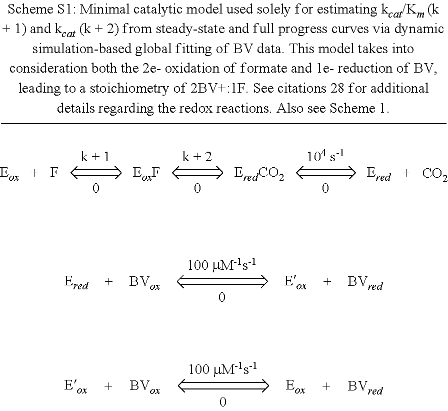

TABLE 4
Steady-state kinetics of DvH-FDH2

| | Electron | Reaction | | Km | k_{cat}/Kₘ | | |
|---|---|---|---|---|---|---|---|
| Enzyme | acceptor | condition | k_{cat} (s⁻¹) | (μM) | (μM⁻¹s⁻¹)* | Replicates | Reference |
| FDH2^{WT} | BV (2 | Anaerobic | 81 | 4 | 20 | 1 | da Silva et |
| | mM) | | | | | | al (2011) |
| FDH2^{Rec} | BV (2 | Anaerobic | 68 ± 5 | 3.5 ± 0.9 | 19.4 ± 5.2 | 3 | This |
| | mM) | | | | | | application |
| FDH2^{Rec} | BV (20 | Anaerobic | 111 ± 16 | 7 ± 3 | 15.8 ± 7.1 | 3 | This |
| | mM) | | | | | | application |
| FDH2^{Rec} | PES/DCPIP | Anaerobic | 220 ± 5 | 5.5 ± 0.5 | 40 ± 4 | 5 | This |
| | | | | | | | application |
| FDH2^{Rec} | PES/DCPIP | Air | 317 ± 14 | 7 ± 1 | 45.3 ± 6.3 | 10 | This |
| | | | | | | | application |

Initial velocities were calculated using ICEKAT, utilizing the first 10-12 seconds of data
*standard error values for k_{cat}/Kₘ estimated according to Johnson (2019)

TABLE 5
Parameters gleaned from full progress curve analysis

| Electron | | Kₘ | k_{cat}/Kₘ | Product | Enzyme | Product |
|---|---|---|---|---|---|---|
| acceptor | k_{cat} (s⁻¹) | (μM)* | (μM⁻¹s⁻¹) | stoichiometry ** | inactivation | inhibition |
| 2 mM BV | 47 ± 2 | 2.1 ± 0.1 | 22 ± 1.1 | 2BV⁺: 1F | No | No |
| (anaerobic) | | | | | | |
| DCPIP | 258 ± 3 | 3.7 ± 0.3 | 69 ± 6 | 1DCPIP:1F | No | No |
| (anaerobic) | | | | | | |
| DCPIP | 354 ± 5 | 4.5 ± 0.4 | 79 ± 6 | 1DCPIP:1F | No | No |
| (air) | | | | | | |

Errors for k_{cat} and k_{cat}/Kₘ via confidence contour analysis implemented in FitSpace Explorer
*Standard error values for Kₘ (calculated from k_{cat} and k_{cat}/Kₘ) estimated according to Johnson (2019)
** F, formate, BV+, reduced benzylviologen

Catalytic Redundancy or Gain of a New Enzyme Function? Exploiting the Peck-Gest Paradigm to Seek Insights Into How FDHs May Have Evolved to Achieve Aerobic Catalysis. Full progress curve analysis establishes that both k_{cat} and k_{cat}/Kₘ are severely underestimated when BV is used as the electron acceptor. It also reveals a preference for the latter viz., whereas DvH-FDH2 favors high-potential acceptors, such as PES/DCPIP or NBT; DvH-FDH1 is highly active with BV²³. Such linkages take on special significance when multiple FDHs encoded by the same organism are compared. Peck and Gest⁹² discovered two types of FDH in Escherichia coli solely based on their preference for artificial electron acceptors—one was linked to phenazine methosulfate (PMS)/DCPIP and its expression was confined to O₂/nitrate-respiring cells while the other was BV-linked and unique to non-respiring cells (reviewed by Stewart⁵¹). It is now clear that Fdh-N is DCPIP-linked,⁷³ and Fdh-H is BV-linked. The third poorly characterized variant of E. coli, Fdh-O, is also DCPIP-linked.⁵⁴ Extending the Peck-Gest paradigm to DvH—only the second microbe for which all three FDHs have now been characterized. It would be predicted that the BV-linked FDH1 is involved in anaerobic respiration and that the DCPIP-linked FDH2 plays a role in aerobic respiration. It has already been established that FDH1 is essential for anaerobic sulfate respiration when formate serves as the electron donor.⁶² Biological function of FDH2 remains to be elucidated. It is herein proven that catalytic parameters derived from viologen-based measurements lack functional information content to make predictions about how well a given FDH would perform under aerobic conditions. Instead, high catalytic performance on BV only guarantees activity under anaerobic conditions. It is possible that confirmation bias has boosted reliance on viologen-based kinetics and stymied efforts to uncover O₂-immune FDHs that can reversibly function in air. This is best exemplified by DvH-FDH2, which exhibits the lowest TN with BV (Table 3) and yet is the most O₂-insensitive of all metallo-FDHs characterized to date from any bacterium. Therefore, biological context must factor critically into future search efforts aimed at discovering air insensitive FDHs.

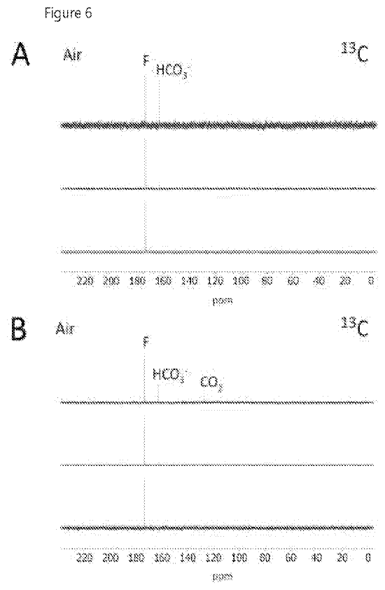

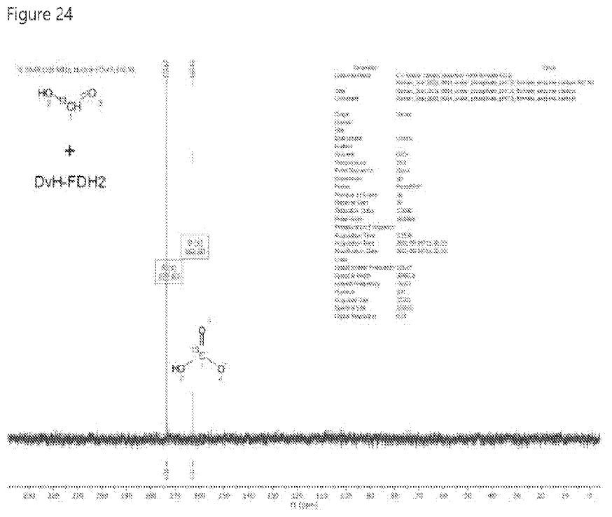

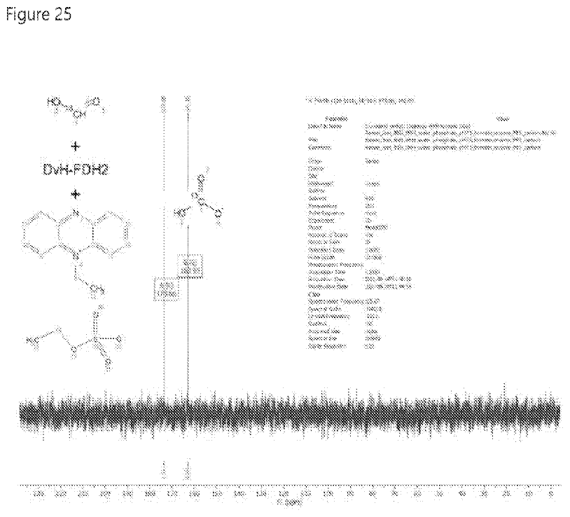

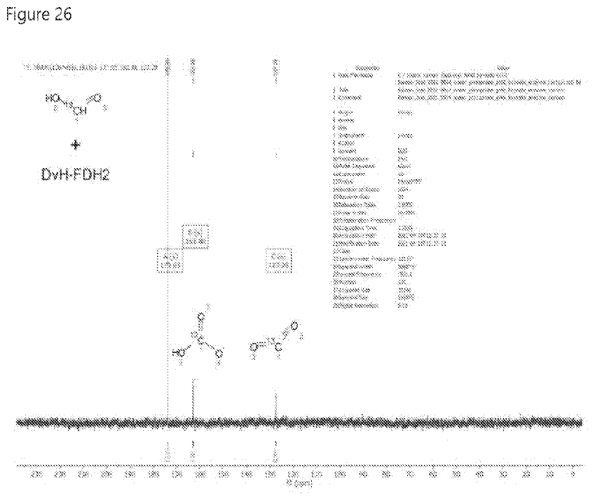

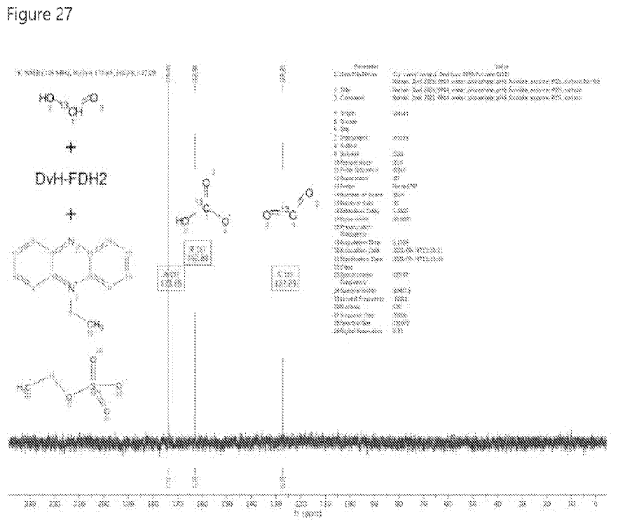

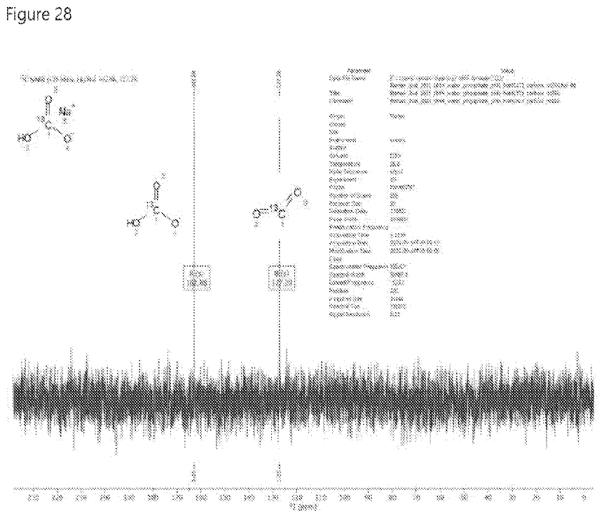

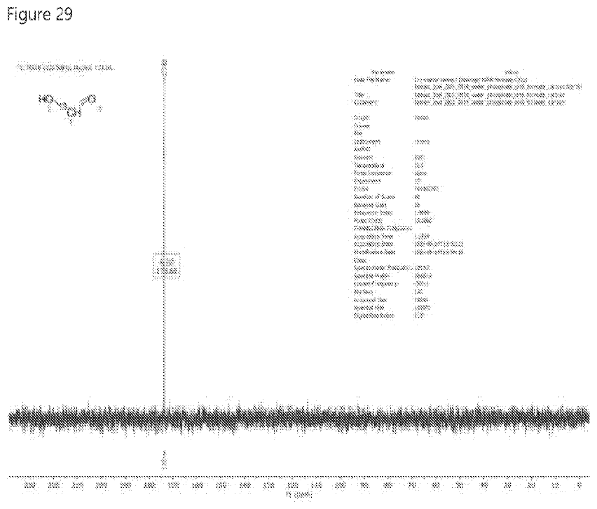

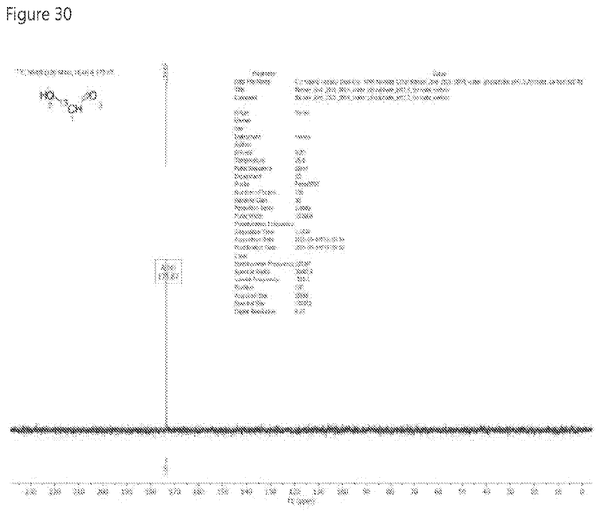

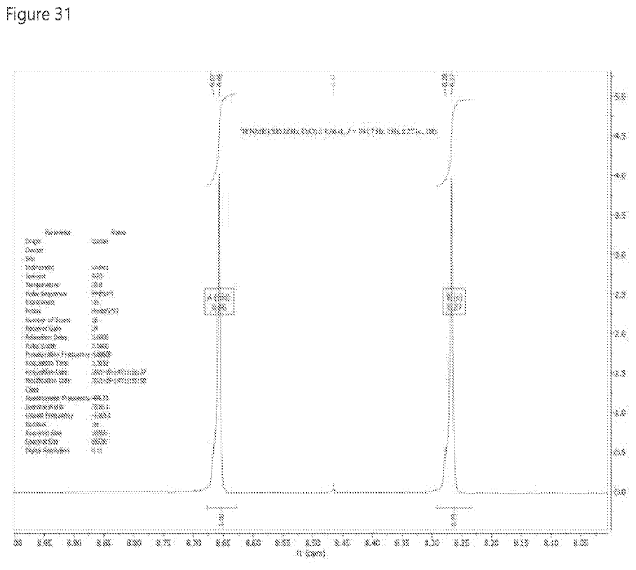

CO2 is the Product of Aerobic Formate Oxidation by DvH-FDH2. Although several metallo-FDHs have been investigated, there is just one report in the literature describing the product resulting from enzymatic oxidation of formate under anaerobic conditions.³¹ In all remaining works, product formation is implied based on the reduction of a natural (NAD⁺) or artificial electron acceptor, which is often BV. Although two different artificial electron acceptors were used in this study, further product analysis in air was studied. At pH 7.5, combining DvH-FDH2 with isotopically labeled ¹³C-formate readily yields a discernible H¹³CO₃⁻ resonance at 162.93 ppm (FIG. 6A, middle and FIG. 24) via ¹³C NMR spectroscopy. Addition of PES to the mix substantially enhances the resonance intensity, indicating that the number of turnovers has increased in the presence of an artificial electron acceptor (FIG. 6A, top; FIG. 25). Whereas DCPIP serves as a proxy for spectrophotometric detection of electron transfer to PES, it is not required for NMR experiments, which directly probe product formation. Because H¹³CO₃⁻ is the dominant anion between pH 7.5-9, ¹³CO₂ was not observed under these conditions. Similarly, at pH 6, both H¹³CO₃⁻ (162.88 ppm) and ¹³CO₂ (127.29 ppm) resonances appear, and their intensities amplify when PES is included (FIG. 6B, middle and top, respectively; FIGS. 26 and 27). As a positive control, it was confirmed that NaH¹³CO₃, in isolation at pH 6, generates H¹³CO₃⁻ and ¹³CO₂ resonances at positions identical to those found with enzymatic formate oxidation (FIG. 28). These chemical shifts agree well with those reported in the literature.^{93 13}C-formate was independently validated by both ¹H and ¹³C NMR spectra. Whereas the latter generates a single resonance at 173.65 ppm (FIGS. 6A and 6B, bottom; FIGS. 29 and 30), J-coupling (˜195 Hz) between the ¹H and ¹³C atoms splits ¹H spectrum of ¹³C-formate into two resonances (8.66 ppm and 8.27 ppm) (FIG. 31). Despite the poor sensitivity of ¹³C NMR spectroscopy⁸² and not optimizing data collection for 5×T₁ (relaxation time), it is herein demonstrated that CO₂ is a product of aerobic catalysis. It was unclear whether HCO₃⁻ formed first and subsequently underwent dehydration to yield CO₂ or whether the latter was produced initially and then hydrated. Only properly controlled ¹⁸O labeling mass spectrometric analysis can help distinguish between the two outcomes.^{31,152} To a first approximation, CO₂ production is rapid under these conditions, suggesting that it is the true product of the reaction.

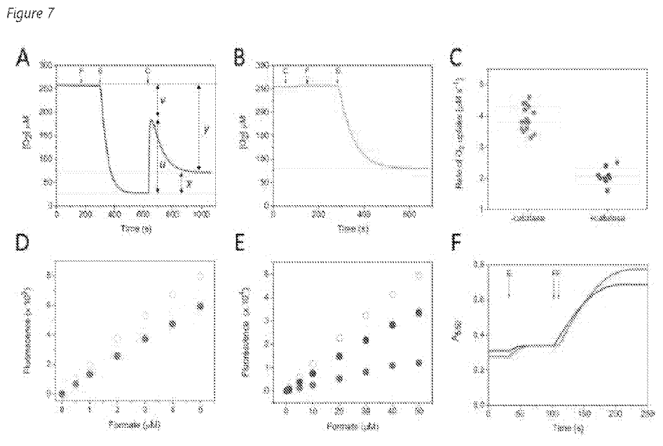

FOX Activity Generates H₂O₂, Enabling Oxygen Insensitivity of DvH-FDH2. A striking characteristic of FDH is that it can catalyze BV reduction in air (See video at pubs.acs.org/doi/suppl/10.1021/acscatal.2c00316/suppl_file/cs2c00316_si_002.mp4 or ndownloader.figstatic.com/files/36617584.). No other FDH is known which is capable of accomplishing this feat. Although transient-blue schlieren appears immediately upon enzyme addition, there is a substantial lag before the reaction mix turns completely blue. This critical observation formed the basis of the hypothesis that O₂ is a cosubstrate for the biocatalyst. To understand how DvH-FDH2 deals with O₂, a Clark-type O₂ electrode was used to determine whether formate oxidation under atmospheric conditions is coupled to O₂ reduction. Addition of enzyme to formate-containing aerobic buffer led to robust O₂ consumption (FIG. 7A). Once the reaction reached equilibrium, catalase was added to assess the nature of products resulting from O₂ reduction. This led to O₂ evolution followed by O₂ uptake until a new equilibrium level was reached, suggesting the production of H₂O₂ via 2e⁻ reduction of O₂ (reaction 3).

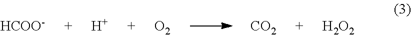

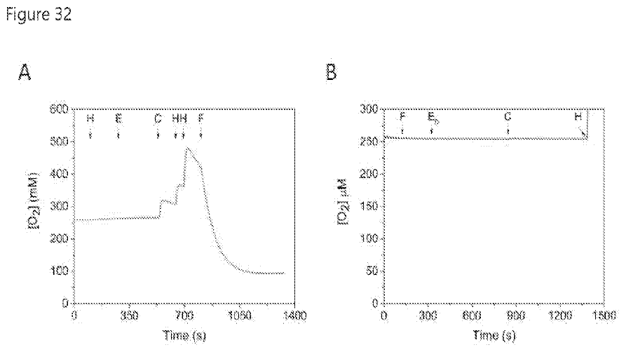

A calculation of the electron flux that leads to H₂O₂ formation was attempted.^{95,96} The x/y value in FIG. 7A would imply that roughly one quarter of the electrons from formate were ending up in H₂O₂. However, this is likely to be an underestimate because formate was in large excess and was continuing to be oxidized post dismutation of H₂O₂ by catalase, manifesting as the second O₂ uptake step. Instead, if considering the u/v value, ˜80% of the electron flux goes towards hydrogen peroxide generation. To resolve this uncertainty, the kinetics approach developed by Lu and colleagues was pursued.⁹⁸ By comparing the initial velocities of O₂ uptake in the absence (FIG. 7A) and presence (FIG. 7B) of catalase, it was found that O₂ uptake was 50% lower in the latter (FIG. 7C). Also, the rates did not vary significantly between pH 6.0 and 8. This outcome suggested that H₂O₂ was the sole product of O₂ reduction during aerobic formate oxidation. Appropriate controls were built into the experimental design for evaluating alternate endpoints. H₂O₂ addition in the absence of exogenous catalase showed that DvH-FDHs lacks native catalase activity of DvH-FDH2 (FIG. 32A). To rule out the possibility of abiotic O₂ consumption, DvH-FDH2 was heat denatured and subjected to oximetry. Neither O₂ uptake nor H₂O₂ generation was found (FIG. 32B). Moreover, inclusion of 1 mM EDTA minimized artifacts arising from transition-metal contaminants. To eliminate the possibility that atmospheric O₂ leaked into the reaction chamber during oximetry, two orthogonal approaches were pursued to quantify H₂O₂. First, horseradish peroxidase (HRP)-catalyzed formation of fluorescent resorufin from H₂O₂ and Amplex red (AR) was monitored. It was observed that for every mole of formate oxidized, roughly 0.75 mol of H₂O₂ was produced during aerobic DvH-FDH2 catalysis (FIG. 7D).

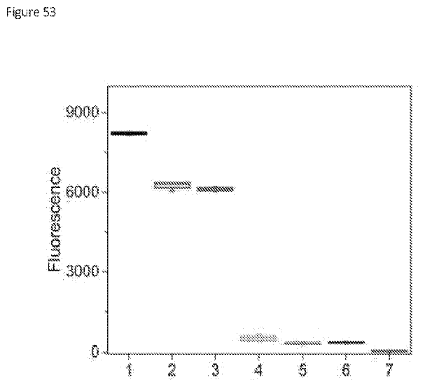

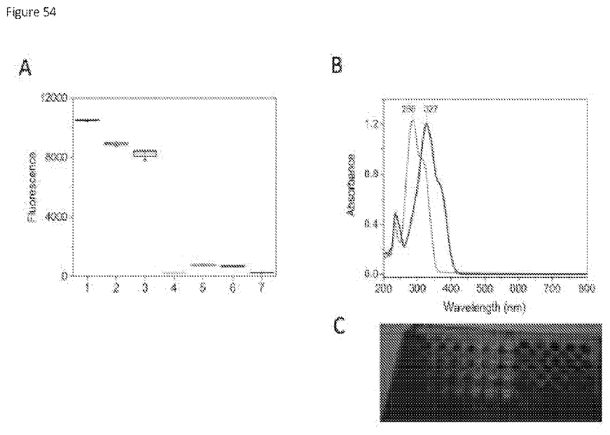

Inclusion of catalase abolished the fluorescence signal, and the denatured enzyme failed to yield H₂O₂(FIG. 53). However, the inability of the AR assay to quantify H₂O₂ beyond M (without having to dilute the samples and remeasure) made it impossible for us to investigate the consequences of O₂ reduction at formate concentrations approaching 10-20 Kₘ. Furthermore, despite being considered the gold standard, this assay is prone to artifacts.^{99,100} For example, interferences stem from the interaction between redox enzymes and resorufin.¹⁰¹ To overcome these limitations, a method independent of HRP and AR was used. Here, the direct reaction of nonfluorescent coumarin-7-boronic acid (CBA) with H₂O₂ was followed, leading to the production of fluorescent 7-hydroxycoumarin (COH).¹⁰² Although this reaction is slow (kₒₙ 1.5 M⁻¹ s⁻¹), the assay is linear over a much broader range of H₂O₂. Therefore, H₂O₂ production was quantified during aerobic DvH-FDH2 catalysis, varying formate concentrations between 1 and 10 Kₘ. It amounted to 64±6% and did not show significant variation when higher enzyme concentrations were used (FIG. 7E). Catalase addition eliminated the signal completely, confirming that H₂O₂ is indeed the major product of O₂ reduction by DvH-FDH2 (FIG. 54).

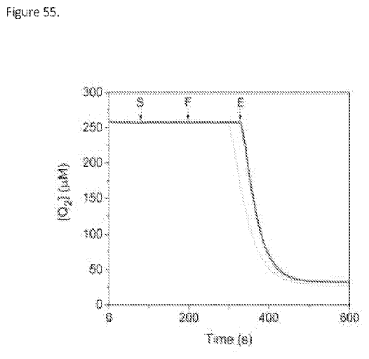

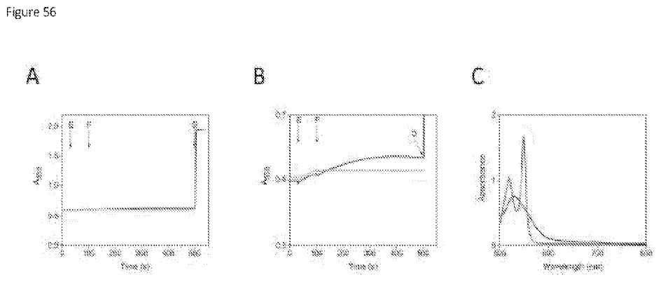

Next, superoxide (O₂^{·-}) generation¹⁰³ by DvH-FDH2 was assessed. Because addition of superoxide dismutase (SOD) had a negligible effect on both quantification (FIGS. 53 and 54) and oximetry (FIG. 55), the reduction of partially acetylated cytochrome c was probed. The advantage of using the latter is that it is still reducible by O₂^{·-} but not susceptible to interferences arising from oxidase or reductase activities when unmodified cytochrome c serves as the substrate.¹⁰⁴ Significant reduction was not observed, implying that either O₂^{√-} remained bound to a metal/donor during catalysis or that it did not diffuse out of the site (FIG. 56; reaction 4). As direct two-electron transfer to O₂ is not possible, one-electron reduced O₂^{·-} must serve as the precursor to H₂O₂ (reaction 5)^{105,106}

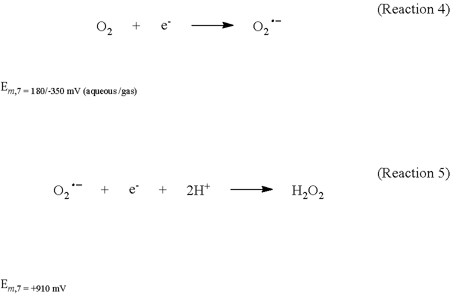

Taken together, these results establish for the first-time FOX activity of a metallo-FDH. Based on IUPAC-IUB nomenclature,¹⁰⁷ the term “oxidase” (EC 1.1.3) is reserved for enzymes, which utilize O₂ as the electron acceptor. In this case, formate oxidation is coupled to 2e⁻ reduction of O₂ by DvHFDH2, resulting in 65 to 100% H₂O₂ production (reaction 3). It is projected that roughly 0-35% O₂ is reduced to H₂O by a 4e process (reaction 2). From a mechanistic perspective, this is reminiscent of how O₂ insensitivity is achieved in some [NiFe]-hydrogenases.¹⁰⁸ However, given the high level of difficulty associated with detecting and quantifying H₂O, only a handful of studies have been performed using redox enzymes.^{98,101,109,110} Ongoing ¹⁷ONMR studies should allow clarification of the stoichiometry of O₂ reduction.

Co-occurrence of FOX and FDH Activities Is a Consequence of Metal-Based Electron Bifurcation. To better understand the redox biochemistry of DvH-FDH2, the results (FIGS. 5-7) were used to advance a working model comprising reductive (reaction 6) and oxidative (reaction 7) half-reactions, which are reminiscent of counterparts in flavoprotein oxidase/dehydrogenase^{111,112} catalysis.

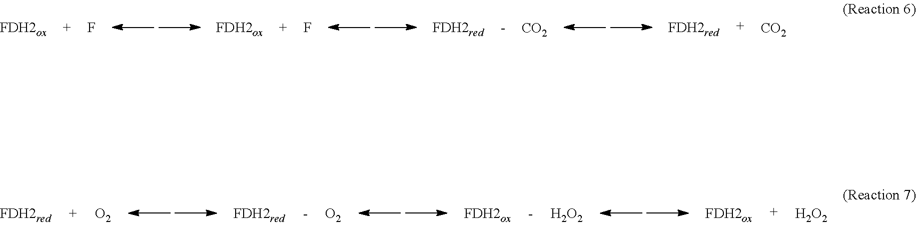

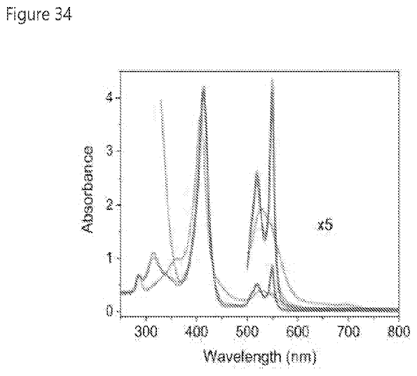

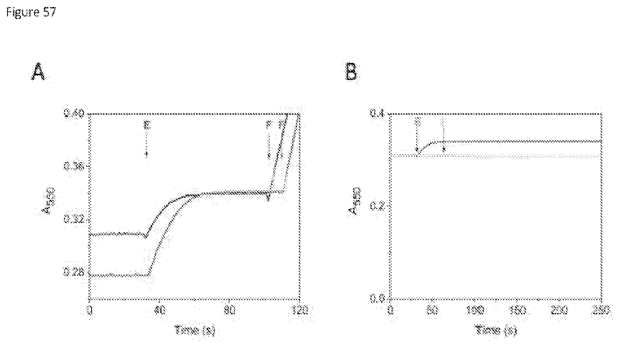

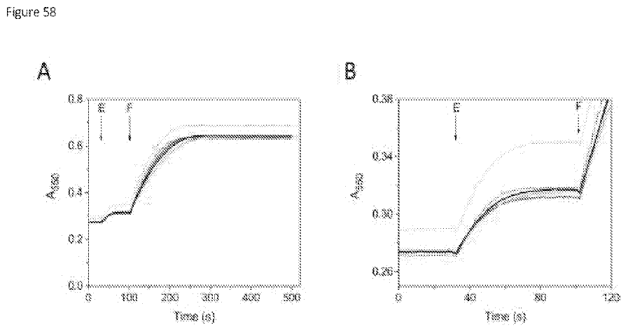

Here, “ox” and “red” represent the oxidized and reduced forms of the enzyme, respectively, while F denotes formate. When DvH-FDH2 couples formate oxidation to the reduction of electron acceptors other than O₂, it functions as a dehydrogenase (reaction 6). The kinetic (FIG. 5) and product (FIGS. 6 and 7) analyses illustrate that FOX activity (reaction 7) does not interfere with the latter function and requires the generation of FDH2red via the FDH branch. Nevertheless, it was unknown if this pattern holds when a macromolecule serves as an electron acceptor. Because the central hypothesis rests on the assumption that FOX activity is likely to preserve electron transfer to natural high-potential acceptors, this was tested directly. Since a gene encoding an 11-heme cytochrome c (uhc; DVU2483) is located immediately adjacent to fdh2 in DvH,⁶⁹ it was investigated whether DvH-FDH2 reduces native equine cytochrome c (E_{m,7}=+260 mV¹¹³) in the presence of formate. Whereas stoichiometric reduction occurred under anaerobic conditions, ˜80% underwent reduction in air (FIG. 7(F)). However, the initial rates remained invariant (FIG. 57). Doubling the formate concentration resulted in near-stoichiometric reduction in air (FIG. 34). Moreover, inclusion of SOD or catalase did not result in a noticeable difference (FIG. 58). Under the conditions employed, dissolved O₂ (257 M) is roughly an order of magnitude higher in concentration than cytochrome c. Yet, electrons are readily delivered to the latter. Next, the stoichiometry of H₂O₂ production under these conditions (FIG. 7E) was investigated. Although interference from peroxidase activity^{114,115} of cytochrome c led to a significant underestimation (˜30%), control measurements designed to quantify H₂O₂ consumption by cytochrome c alone allowed estimation of the stoichiometry. Thus, for every formate oxidized, roughly 0.7H₂O₂ was released, illustrating that the oxidase and dehydrogenase branches are not competing for electrons. It remains to be seen how Uhc impacts the outcome. NMR^{98,99} investigations aimed at direct H₂O₂ quantification are underway. In sum, these results prove for the first time that oxidase and dehydrogenase activities not only coexist within the DvHFDH2 scaffold but are simultaneously enabled in air. Below, a conceptual framework is provided for interpreting these results.

Despite nearly a century of research, the precise mechanism(s) of FDH catalysis remains incompletely understood. Indeed, several structures have been determined,^{15,23,116,117} but none reveal a bound formate or CO₂ near the tungstopterin cofactor (Wco). Nor do they consider radiation-induced photoreduction of protein crystals during X-ray data collection,¹¹⁸⁻¹²⁰ thus raising questions about the significance of structures labeled “oxidized” or “reduced”. Not surprisingly, five mechanisms have been advanced to explain FDH catalysis with hydride (2e⁻ and a H⁺) transfer from formate to the Mo/W center being the most favored.^{13,121} This paucity of essential information has led us to rationalize concurrent FDH and FOX reactions from first principles. Thus, the rapid equilibrium model¹²² is combined with Moser-Dutton formalism^{123,124} to help interpret the vagaries of electron transfer processes in DvH-FDH2. At the outset, six electrons would be needed for complete enzyme reduction-two for Wco (reactions 8 and 9) and one each for the four [4Fe-4S] clusters (reaction 10; FIGS. 3 and 4).

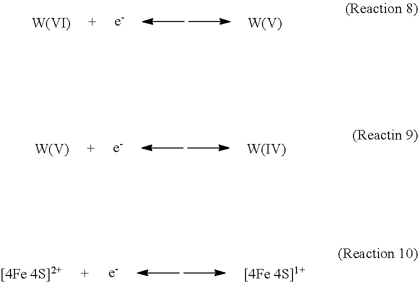

Since formate is a two-electron donor, it would take 3 mol of the substrate to fully reduce all the redox sites in DvH-FDH2 (reaction 6). When O₂ reacts with the completely reduced enzyme, nearly 3 mol of H₂O₂ are produced (reaction 7). Therefore, transfer of electrons from formate to Wco likely constitutes the rate-limiting step. Once the latter is accomplished, electrons are expected to rapidly equilibrate within the system via intramolecular transfer events. The iron-sulfur clusters are expected to function as electron sinks, facilitating the substrate to repeatedly inject electrons into Wco when cytochrome c is also involved. This scenario would require modification if both formate oxidation and O₂ reduction were to occur at Wco. In any case, where electron flux is concerned, the redox equilibria would be expected to generate 48 unique enzyme microstates at varying levels of reduction. As the intramolecular electron equilibration is expected to be rapid, the reactivities of formate and O₂ with the enzyme will be determined by the relative reduction potentials of their respective product-bound states (reactions 6 and 7). Meanwhile, understanding the mechanism of O₂ activation is central to the question of how FOX activity is enabled. There are two possibilities. First, Wco may be directly involved. As precedent, the Mo-center of plant sulfite oxidase generates¹²⁵ O₂^{·-} as a product. Second, the large subunit [4Fe-4S] cluster is a potential candidate for producing H₂O₂ via reactions 11 and 12.¹²⁶

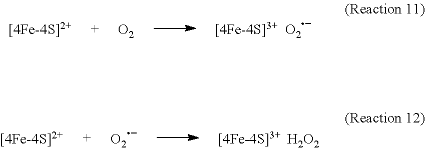

Such sequelae are thought to irreversibly damage the clusters,¹⁰⁵ and there is no evidence for this occurring in DvH-FDH2. Alternatively, by analogy with O₂-insensitive [NiFe] hydrogenases, one of the [4Fe-4S] clusters of FDH2 could reduce O₂ without being damaged during the process. Based on the structural prediction for DvHFDH2, it is expected for the O₂ reduction site to be within the coordination distance of either W or the [4Fe-4S] cluster ligated to the large subunit. Extending this logic to cytochrome c (a one-electron acceptor; reaction 13),

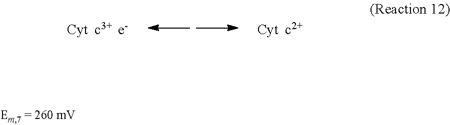

a plausible electron transfer path can be charted: formate→W→[4Fe-4S]→Cyt c.

It is believed that both Uhc and equine cytochrome c target a site near the last [4Fe-4S] cluster of the small subunit. Thus, in the context of a macromolecular electron acceptor, formate oxidation and cytochrome c reduction sites are separated by ≥50 Å, which is beyond the tunneling distances where Moser-Dutton ruler operates (ca. 8-20 Å).

Thus, it is unambiguous that there are at least two thermodynamically favored electron transfer paths in DvH-FDH2 to facilitate the simultaneous reduction of cytochrome c and O₂ (reaction 14).

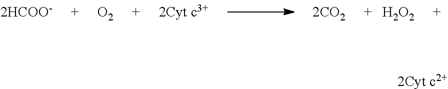

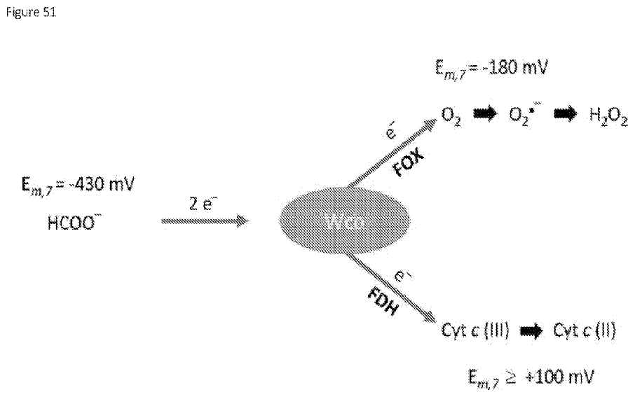

A mechanism is shown to explain how this is accomplished (FIG. 51). Upon receiving two electrons from formate, Wco splits the pair, transferring one electron to O₂ while sending the other to cytochrome c. Based on reaction 4, the electron transfer path culminating in O₂ reduction is designated as the exergonic low-potential branch. As only H₂O₂ is detected as the product, Wco must initiate a second 1e⁻ transfer to FDH2-bound O₂^{·-}. In a similar vein, reaction 13 helps define the exergonic high-potential branch as the 1e⁻ transfer to cytochrome c. This type of pair splitting has important precedents in bioenergetics and is frequently referred to as electron bifurcation (EB). Some have used the term cooperative bidirectional one-by-one electron transfer to distinguish it from noncooperative counterparts, as well as unidirectional events.¹²⁷ EB was originally proposed to rationalize mitochondrial quinone-based (QB) energy coupling.^{128,129} The present-day literature is largely focused on flavin-based (FB) EB.¹³⁰⁻¹³² Besides QBEB and FBEB, metal-based (MB) EB has also been proposed.^{134,135} Conceptualization of FBEB has also been studied.¹³³ Since DvH-FDH2 activity does not require quinone or flavin, it was reasoned that MBEB could potentially explain how concurrent 1e⁻ transfers are initiated. Because the ability of a metal center to mediate both 1e⁻ and 2e⁻ transfers is a conditio sine qua non for MBEB,¹³⁴ Wco is favored as the bifurcating site, eliminating 1e⁻ transferring [4Fe-4S] clusters from consideration. Originally, inverted (or crossed) potentials were considered essential for the strictly coupled EB reactions.^{132,134} This would imply that the first electron egressing from Wco is of the high-potential type intended for cytochrome c. Conversely, the second electron must exhibit low potential and readily picked up by O₂.

However, new computational models predict that sites with normally distributed (uncrossed) potentials are EB-competent as well.¹³⁶ Considering that proton-coupled electron transfer¹³⁷ must occur during both formate oxidation and O² reduction, it is posited that inverted potentials likely dominate at the Wco site during catalysis. Consistent with this assessment, an extremely weak W(V) EPR signature is observed with aerobically or anaerobically formate-reduced DvH-FDH2 (FIG. 4). Even though W(V) intensity is expected to be weak [circa one-third of the total signal with W(VI) and W(IV) being EPR-silent¹³⁸], it has been shown that it is sufficiently enhanced when the enzyme is reduced with a slow 1e⁻ reductant (dithionite) for 12 h under nonphysiological (anaerobic) conditions. Precisely how the protein scaffold controls Wco reduction potentials in DvH-FDH2 remains to be studied. Nor is anything known about the noninnocent roles of dithiolene and pterin. Equally unclear is why Wco is catalyzes aerobic formate oxidation when its services are thought to be best suited for facilitating very low-potential reactions.⁷⁷

The MBEB mechanism is consistent with what has already been advanced in the context of QBEB or FBEB. Insofar as the gold-standard [butyryl-CoA dehydrogenase (Bcd)-electron transfer flavoprotein (EtfAB) complex devoid of iron-sulfur clusters]¹³⁹ undergoes FBEB in air utilizing two exergonic branches,¹⁴′ allowing O₂ to pick up the low-potential electron destined for ferredoxin,¹⁴¹ DvH-FDH2 engages MBEB to achieve a similar outcome (FIG. 51). A key difference, however, is that the FDH enzyme generates H₂O₂ without releasing the 1e⁻ product, O₂^{·-}. Nonetheless, after correcting for H₂O₂ consumption by equine cytochrome c, the product stoichiometry is in the same range as that observed for Bcd-EtfAB. Indirect quantification of H₂O₂ and/or O₂^{·-} are fraught with complications, which could potentially contribute to stoichiometric variabilities documented for ferredoxin-associated Bcd-EtfAB.¹⁴¹ Other factors are also known to diminish stoichiometry of coupling in bifurcating [FeFe]hydrogenases.²⁹ Finally, despite the current focus on reversible EB,¹⁴² irreversibility has entered the discussion.¹³⁶ O₂ reduction enabled during FBEB (Bcd-EtfAB)^{140,141} and MBEB (this work) underscores that irreversible EB deserves consideration. The oft-invoked short-circuiting¹⁴³ or toxicity of reactive oxygen species to all anaerobes¹⁴¹ fails to explain why O₂^{·-} production is prevalent in most flavoenzymes¹⁴⁴ or how some organisms that conserve energy via FBEB not only thrive in the presence of O₂ but also utilize H₂O₂ as a terminal electron acceptor. Regardless, DvH-FDH2 has evolved O₂ insensitivity by enabling MBEB.

System-level consequences are often overlooked when the focus is restricted to an enzyme or a mechanism thereof. This is true for FDH as well. Even though EB is an energy coupling mechanism, prevailing models cannot explain why the disruption of QBEB¹⁴³ or reduction potentials¹⁴⁵ fails to affect organismal growth and/or electron transfer pathways. Nonetheless, these unexpected outcomes hint that the cellular bioenergetic machinery is poised to seamlessly adapt to fluctuations in signals, circuits, and environmental conditions. Envisioning the big picture, testable hypotheses were carefully considered that emerge from the discovery of an O₂-insensitive FDH. One of these focuses on energetic coupling via aerobic FBEB. To put it in perspective, FBEB is currently thought to be primarily beneficial to strict anaerobes. This confirmation bias stems from the view that electron transfer to ferredoxin is possible only when O₂ is absent. As a result, clostridia are thought to stop benefitting from this mechanism once they encounter oxygenic conditions.¹⁴¹ However, FBEB-capable human pathogens, such as Clostridioides difficile (formerly Clostridium difficile), retain growth in the presence of 2% O₂. Nothing is known about whether FBEB contributes to the cellular energy expenditure in these oxic niches. If indeed Azotobacter vinelandii¹⁴⁷ (an obligate aerobe with a hypoxic intracellular milieu) is a candidate for FBEB, so should be C. difficile. More importantly, it has been demonstrated that O₂-evolving cyanobacteria utilize a mode of growth, which requires electron transfer to ferredoxin under aerobic conditions.¹⁴⁸ Although the authors do not invoke FBEB, those findings advance the hypothesis that O₂-insensitive FDHs support energy coupling via aerobic FBEB. DvH-FDH2 is localized to the periplasm and, therefore, is unlikely to encounter ferredoxin or NAD⁺. However, O₂-insensitive metallo-FDHs resident in the cytosol are likely well equipped to initiate FBEB under aerobic conditions. It is proposed that Methylosinus trichosporium OB3b FDH36,37 is a suitable candidate for exploring aerobic FBEB. It has also been noted that the inverted flavin potentials associated with this cytosolic enzyme³⁶ may render it FBEB-competent, but such arguments did not take the following into consideration.¹⁴⁹ First, M. trichosporium (Mt) is an aerobic methanotroph, requiring O₂ and methane for growth. Second, addition of formate to its growth culture leads to an increase in intracellular NADH levels. Third, in vitro reconstitutions have implied that NAD⁺-dependent FDH from Mt must reduce ferredoxin first before nitrogenase activity can be enabled.¹⁵⁰ Fourth, unpublished results¹⁵¹ show that flavin semiquinone is highly destabilized in Mt-FDH, with no more than 15% being detectable by EPR. Collectively, these observations engender confidence in the feasibility of aerobic FBEB. Since Gottschalkia acidurici (formerly Clostridium genus) FDH has already been shown to mediate FBEB under anaerobic conditions,¹⁵² it should be possible to properly assess how its Mt counterpart performs under aerobiosis. Empirical results from such investigations would inform the extent to which aerobes and facultative anaerobes exploit FBEB to meet their energy demands.

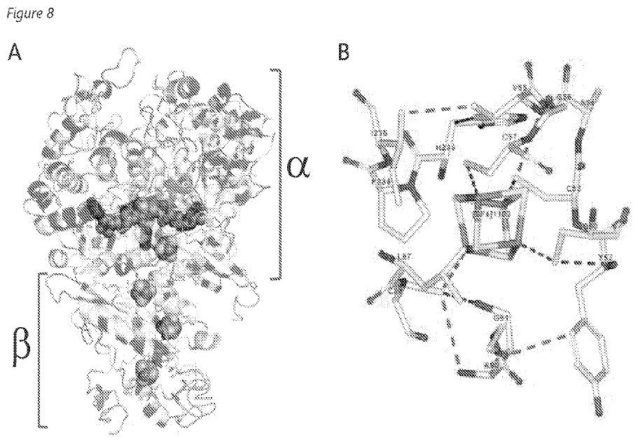

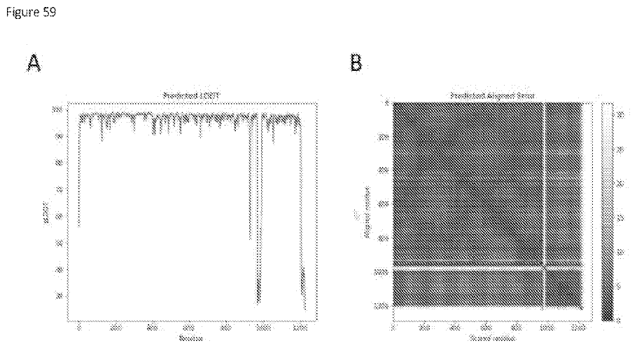

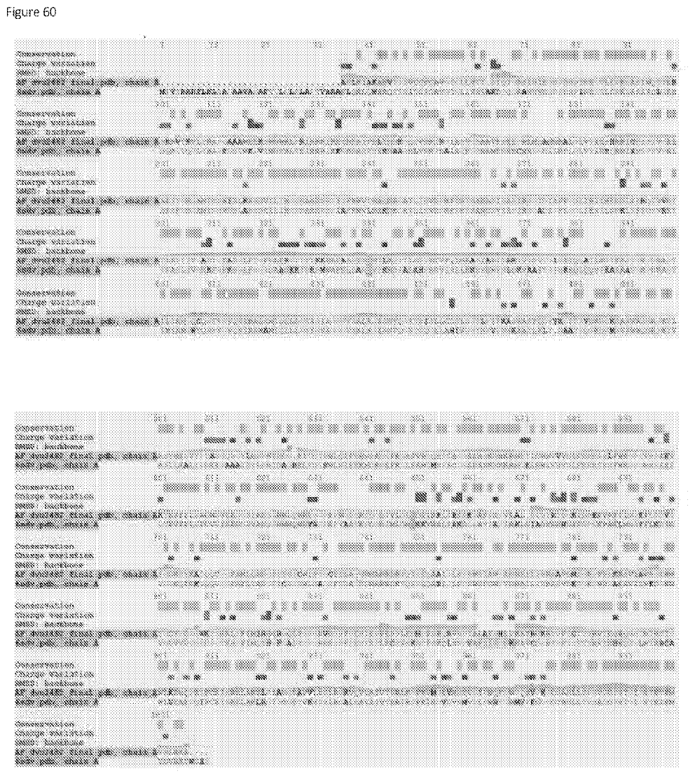

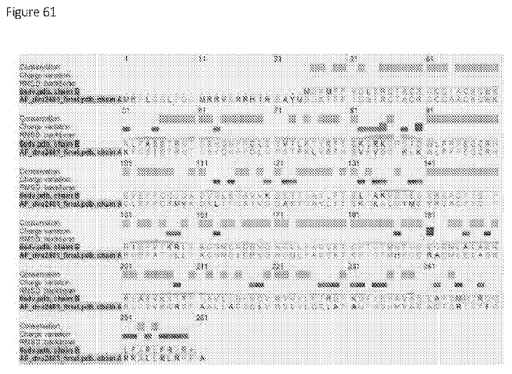

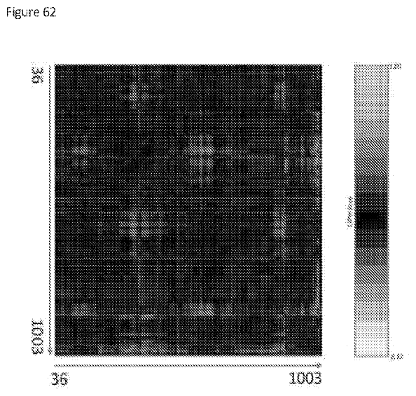

Molecular Basis of DvH-FDH2 O₂ Insensitivity. In their phylogenetic analysis, Oliveira et al²³ began with over 6000 FDH sequences and reduced it by an order of magnitude in an effort to understand how the variability impacts catalytic mechanism and O₂ stability. Here, the sequence space was narrowed to just two closely related paralogs—one of these (DvH-FDH1) is unable to achieve catalysis in the presence of O₂ while the other (DvH-FDH2) thrives in air. To gain atomic insights, a de novo structure of the latter was built using AlphaFold2.1¹⁵³ (FIG. 8A). Preexisting structure templates were not used to model the structure. The resulting atomic coordinates (without cofactors) include confidence metrics (predicted local distance difference test, pLDDT) at the single residue level wherein higher scores on a scale of 1-100 represent greater confidence. FIG. 59(A) shows that the heterodimeric structure of DvH-FDH2 is modeled with high confidence; the bulk of the polypeptide chain displays pLDDT scores>90. Similarly, the predicted aligned error (PAE; color saturation found at any x, y coordinate in FIG. 59(B)) is a metric of how well a residue is positioned and oriented. Here too, very low PAE values are seen for both subunits. Independently, the structures of the two subunits were predicted and compared with the heterodimeric counterpart. Both approaches yielded very similar results. DALI¹⁵⁴ confirms that the tertiary folds of large and small subunits are superimposable on their DvH-FDH1 counterparts with a rootmean-square-deviation (RMSD) of 1.4 Å (953 Ca atoms; Zscore 58.2; 61% identity) and 1.0 Å (213 Cα atoms; Z-score 35.9; 63% identity), respectively. Backbone RMSD variations are shown in FIGS. 60 and 61). Similarly, the RMSD between DvH-FDH1 and DvH-FDH2 heterodimers is 1.37 Å (952 Cα atoms). After completing these validations, computed a residue-residue (RR) distance map was computed to identify the regions of major variation between FDH2 and FDH1 (FIG. 62).

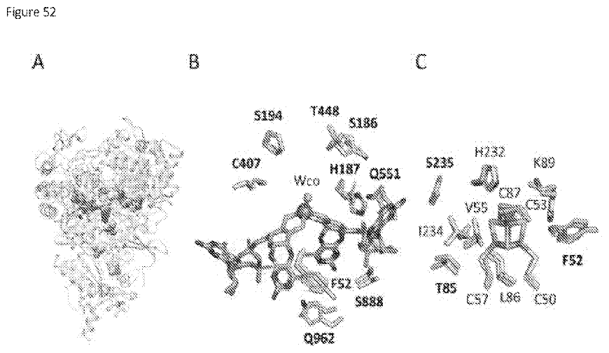

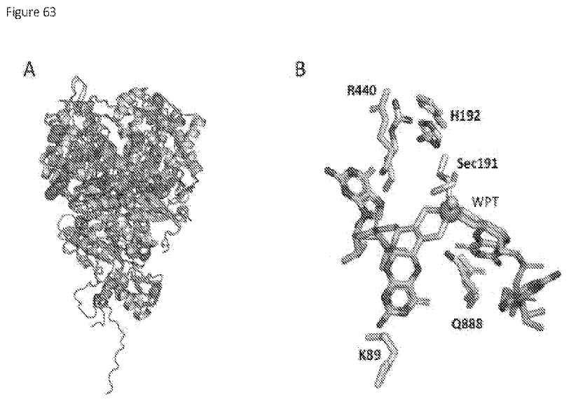

Although the active-site residues are largely conserved between FDH2 and FDH1, there are several differences in the vicinity of the tungsten center (compare FIGS. 52(B) and 63(B)). Specifically, two highly conserved residues²³ have been substituted in FDH2. The introduction of S186 and H187 are noteworthy because they replace H187 and Q188, respectively, in FDH1. These changes are likely to influence Sec reactivity.

[4Fe-4S] clusters are prone to oxidative damage,^{105,126} and enzymes harboring them would be expected to be inactivated by H₂O₂ generated during aerobic catalysis.¹⁵⁷ However, this does not happen with DvH-FDH2. As a corollary, cellular experiments with the Campylobacter group of bacteria have shown that they harbor an FDH capable of producing H₂O_{2.}¹⁵⁸⁻¹⁶⁰ In these organisms, H₂O₂ functions as a terminal electron acceptor in respiration.^{95,96,161} E. coli capitalizes on H₂O₂ in a similar fashion.¹⁶² Therefore, the environment of the active-site proximal [4Fe-4S] cluster was examined to glean insights. Strikingly, there are three substitutions (Y52F, S85T, and A236→S235) within a 5 Å radius (FIG. 52C). This is relevant because in some O₂-tolerant [Ni—Fe]-hydrogenases, introduction of a polar residue near catalytic metal clusters has been shown to improve O₂ resistance.¹⁶³ One or more of the following precedents may inform how O₂ insensitivity and/or EB evolved in this system: (1) whereas group 1d O₂-tolerant [Ni—Fe]-hydrogenases, which also reduce O₂ to H₂O₂ and H₂O,^{101,164} generate an unusual [4Fe-3S] cluster, group 5d counterparts do not; (2) a canonical [4Fe-4S] cluster in another [NiFe] hydrogenase undergoes redox-dependent structural changes,¹⁶⁵ poising it in a protected state until the next catalytic cycle; (3) second coordination sphere effects; and (4) local or remote conformational fluctuations at the protein level that could offer protection from attack by oxidants.

Impact The findings reported here have broad utility in disparate fields of research. The current state of the art in formate/air biofuel cells is limited to mediated electron transfer because O₂-sensitive metallo-FDHs need protection from redox polymer films to operate.¹⁶⁶ Consequently, a “true” formate/air biofuel cell is yet to be fabricated. DvH-FDH2 should be able to work in the absence of protective matrices and power biofuel cells via direct electron transfer. DvH-FDH2 should be able to work in the absence of protective matrices and power BFCs via direct electron transfer. All metallo-FDHs are thought to be capable of catalyzing the CO₂ reduction reaction (CO2RR).¹⁴ However, there is no known biocatalyst that accomplishes CO2RR in air.¹⁶⁷ Also, it is not possible to test this reaction with low-potential artificial electron donors (e.g., MV) because they react with O₂.¹⁶⁸ Since the redox cofactors of DvH-FDH2 are O₂-insensitive, there is a high probability of exploiting bioelectrocatalysis to run the reverse reaction aerobically. Considering that an O₂-insensitive [NiFe]hydrogenase has been successfully shown to perform aerobic bioelectrocatalysis,¹⁶⁹⁻¹⁷¹ it should be possible to achieve the same with DvHFDH2. It is anticipated that redox bifurcation would also be in operation during aerobic CO2RR. If this prediction holds true, it will facilitate deeper understanding of a bifurcating site in the context of two fundamentally distinct reaction trajectories-formate oxidation and CO2RR. Photoexcited CdS nanodots¹⁷² offer an independent strategy for injecting electrons into the enzyme's active site. In conjunction with time-resolved infrared spectroscopy,¹⁷³ CO2RR can be probed in greater detail. Surprisingly, despite the distinctive IR signature of CO₂,¹⁷⁴ only two relevant enzymes have been investigated using IR spectroscopy in the past five decades.^{175,176} Structure-function relationships of DvH-FDH2 should inform tunability of catalytic bias,¹⁷⁷ limits to electrocatalytic reversibility,¹⁷⁸ and design of biomimetic metallosynthetics.¹⁷⁹ Aerobic formatotrophs couple formate oxidation (reaction 1) to O₂ reduction,^{8,57} generating an energy equivalency of about 1.25 V. The ability to aerially manipulate DvH-FDH2 will enable strategies for shedding light on unusual bioenergetics.

### III. Examples

### Example 1: Construction of fdh Deletion Strains

The naming schemes for the fdh genes follow the convention established previously⁶⁸. DvH deletion strains (Table 6) were constructed using methods already described^{191,200}. Briefly, for the deletion of each predicted operon, two plasmids were constructed: one to create a marker-exchange deletion and another to remove the marker. Both plasmids are suicide vectors and require at least one homologous recombination event to occur to provide the selectable phenotypes. A phenotypic screen was performed to determine if a double recombination event took place, thereby increasing the likelihood of choosing isolates that had the desired genotype. Each vector contained a cloned copy of at least 300 bp upstream and a similar DNA region downstream of the operon targeted for deletion that were captured in a vector backbone containing the pUC origin of replication and a gene conferring spectinomycin-resistance. The plasmids were constructed by the sequence and ligation independent cloning (SLIC) technique²⁰¹ with amplicons obtained from PCR using the primers found in Table 7 (Integrated DNA Technologies, Coralville, IA) and the Herculase II DNA polymerase (Life Technologies, Grand Island, NY). For the marker-exchange plasmids, the two DNA regions up- and down-stream are separated by an artificial, two-gene operon including aph(3′)-IIa (conferring antibiotic resistance to 50 μg kanamycin/mL in E. coli and 400 μg G418/mL in DvH) and the counter-selectable marker uracil phosphoribosyltransferase (upp, DVU1025) genes. The marker-exchange plasmids were introduced by electroporation into a strain containing a deletion of the upp gene and the operon to be deleted. The transformed DvH cells were allowed to recover overnight at 34° C., as previously described²⁰⁰. The cells were then grown for 3-5 days on solidified MO medium supplemented with yeast extract (Y), lactate (L), and sulfate (S4) (hereafter referred to as MOYLS4 medium)²⁰² containing G418 to select for transformants. Single isolates were screened for sensitivity to 100 μg spectinomycin/mL (consistent with the double homologous recombination event), sensitivity to 40 μg 5-fluorouracil/mL (5FU^{S}; to ensure the counter-selection of 5FU resistance (5FU^{R}) would be effective) and maintenance of resistance to G418. A putative marker-exchange deletion isolate was then chosen and transformed with the marker-less deletion plasmid, as described above. The transformed cells were recovered, plated on medium containing 5FU and the three phenotypic markers again screened. For the marker-less deletion isolates, however, isolates were selected that were 5FU-resistant and G418-sensitive showing that the marker exchange cassette had been removed from the cell by double homologous recombination. Up to three isolates with the desired antibiotic-resistance phenotype were further analyzed by Southern blot. Once confirmed, one of these isolates was chosen as the marker-less deletion mutant.

TABLE 6
Strains and plasmids used in this study

| Strain or plasmid | Genotype and relevant features | Source |
|---|---|---|
| Desulfovibrio vulgaris | | |
| strains | | |
| Desulfovibrio vulgaris | Wild-type strain, ATCC 29579 | ATCC |
| Hildenborough | | |
| JW710 | Desulfovibrio vulgaris | Keller et al., 2009 |
| | Hildenborough Δupp 5FU^{r} | |
| JW2103 | JW710 Δfdh2 | This application |
| JW2109 | JW710 Δfdh3 aph(3′)-IIa: upp | This application |
| | G418^{r} 5FU^{s} | |
| JW2111 | JW710 Δfdh3 | This application |
| JW2115 | JW710 Δfdh1 | This application |
| JW2117 | JW710 Δfdh2 Δfdh3 | This application |
| JW2120 | JW710 Δfdh3 Δfdh1 aph(3′)- | This application |
| | IIa: upp G418^{r} 5FU^{s} | |
| JW2121 | JW710 Δfdh3 Δfdh1 | This application |
| JW2123 | JW710 Δfdh1 Δfdh2 | This application |
| JW2126 | JW710 Δfdh3 Δfdh1 Δfdh2 | This application |
| | aph(3′)-IIa: upp G418^{r} 5FU^{s} | |
| JW2127 | JW710 Δfdh3 Δfdh1 Δfdh2 | This application |
| CSR21210 | JW2121 transformed with | This application |
| | pJEG127 | |
| CSR21271 | JW2127 transformed with | This application |
| | pJEG132 | |
| Escherichia coli strains | | |
| α-select | deoR endA1 recA1 relA1 gyrA96 | Bioline |
| | hsdr17(rₖ− mₖ+) supE44 thi-1 | |
| | Δ(lacZYA-argFU169) | |
| | φ80δlacZΔM15 F- λ- | |
| Plasmids | | |
| pCR8/GW/TOPO | plasmid used to amplify pUC-Sp^{r} | Life Technologies |
| | fragment, Sp^{r} | |
| pMO746 | plasmid containing aph(3′)-II: upp | Parks et al., (2013) |
| | 2-gene operon, Ap^{r}, Km^{r} | |
| pMO2100 | plasmid containing upstream and | This application |
| | downstream regions of fdh-2 on | |
| | either side of aph(3′)-II: upp (used | |
| | to construct marker-exchange | |
| | deletion), Sp^{r}, Km^{r} | |
| pMO2102 | plasmid containing upstream and | This application |
| | downstream regions of fdh-2 | |
| | (used to construct marker-less | |
| | deletion), Sp^{r} | |
| pMO2108 | plasmid containing upstream and | This application |
| | downstream regions of fdh-3 on | |
| | either side of aph(3′)-II: upp (used | |
| | to construct marker-exchange | |
| | deletion), Sp^{r}, Km^{r} | |
| pMO2110 | plasmid containing upstream and | This application |
| | downstream regions of fdh-3 | |
| | (used to construct marker-less | |
| | deletion), Sp^{r} | |
| pMO2112 | plasmid containing upstream and | This application |
| | downstream regions of fdh-1 on | |
| | either side of aph(3′)-II: upp (used | |
| | to construct marker-exchange | |
| | deletion), Sp^{r}, Km^{r} | |
| pMO2114 | plasmid containing upstream and | This application |
| | downstream regions of fdh-1 | |
| | (used to construct marker-less | |
| | deletion), Sp^{r} | |
| pMO9075 | Plasmid used for genetic | Keller et al (2011) |
| | complementation and/or gene | Parks et al (2013) |
| | expression in Desulfovibrio | |
| | strains. It contains Kmr gene- | |
| | aph(3′)-II promoter, pGB1, Sp^{r}, | |
| | and RBS. | |
| pJEG127 | pMO9075 containing the | This application |
| | DVU2482-2481 insert | |
| pJEG132 | pMO9075 containing the | This application |
| | DVU2482strII-2481 insert | |

Δfdh1, Δfdh2, and Δfdh3 represent deletion of DVU0586-0588, DVU2481-2485, and DVU2809-2812 operons in DvH, respectively.

TABLE 7
Primers used in this study

| Primer name | Primer sequence | Application |
|---|---|---|
| SpecRpUC-F | CCAGCCAGGACAGAAATGCCTCG (SEQ | Amplification of pUC- |
| | ID NO: 1) | Sp^{r} fragment |
| SpecRpUC-R | ATGTGAGCAAAAGGCCAGCAAAAGGC | Amplification of pUC- |
| | (SEQ ID NO: 2) | Sp^{r} fragment |
| Kan gene Prom | CCGGAATTGCCAGCTGGGGCGC (SEQ ID | Amplification of |
| Nterm | NO: 3) | aph(3′)-IIa and upp |
| | | fragment |
| upp gene Cterm | CTTACTTGGTGCCGAATATCTTGTCGC | Amplification of |
| | (SEQ ID NO: 4) | aph(3′)-IIa and upp |
| | | fragment |
| DVU2481-5-upF | GCCTTTTGCTGGCCTTTTGCTCACAT | Amplification of |
| | CACTCTTGCGCGAGGAAAGC (SEQ ID | upstream region of |
| | NO: 5) | DVU2481-5 |
| DVU2481-5-upR | GCGACAAGATATTCGGCACCAAGTAAG | Amplification of |
| | GGGAAGGCATTAACCGATACTTG (SEQ | upstream region of |
| | ID NO: 6) | DVU2481-5, specific |
| | | for marker-exchange |
| | | plasmid |
| DVU2481-5-dnF | GCGCCCCAGCTGGCAATTCCGG | Amplification of |
| | CCGACTGGATACGCAACACC (SEQ ID | downstream region of |
| | NO: 7) | DVU2481-5, specific |
| | | for marker-exchange |
| | | plasmid |
| DVU2481-5-dnR | CGAGGCATTTCTGTCCTGGCTGG | Amplification of |
| | CCTGTTCGGACTCTCGATGTTC (SEQ ID | downstream region of |
| | NO: 8) | DVU2481-5 |
| DVU2809-12-upF | GCCTTTTGCTGGCCTTTTGCTCACAT | Amplification of |
| | CAGAACCTCATCGCCATGC (SEQ ID NO: | upstream region of |
| | 9) | DVU2809-12 |
| DVU2809-12-upR | GCGACAAGATATTCGGCACCAAGTAAG | Amplification of |
| | TCCTCTCCTTGTTGATGCCCTG (SEQ ID | upstream region of |
| | NO: 10) | DVU2809-12, specific |
| | | for marker-exchange |
| | | plasmid |
| DVU2809-12-dnF | GCGCCCCAGCTGGCAATTCCGG | Amplification of |
| | GGGAATGTCGTCTCACGCAG (SEQ ID | downstream region of |
| | NO: 11) | DVU2809-12, specific |
| | | for marker-exchange |
| | | plasmid |
| DVU2809-12-dnR | CGAGGCATTTCTGTCCTGGCTGG | Amplification of |
| | GTTTCCGGCAAGGTCAAGG (SEQ ID NO: | downstream region of |
| | 12) | DVU2809-12 |
| DVU0586-88-upF | GCCTTTTGCTGGCCTTTTGCTCACAT | Amplification of |
| | TGGGCGTACAGTTCGGTATC (SEQ ID | upstream region of |
| | NO: 13) | DVU0586-8 |
| DVU0586-88-upR | GCGACAAGATATTCGGCACCAAGTAAG | Amplification of |
| | GTGACAAAGCAACGCATCTTGTG (SEQ | upstream region of |
| | ID NO: 14) | DVU0586-8, specific |
| | | for marker-exchange |
| | | plasmid |
| DVU0586-88-dnF | GCGCCCCAGCTGGCAATTCCGG | Amplification of |
| | TCTGCCGAAGAAAGATGCCTG (SEQ ID | downstream region of |
| | NO: 15) | DVU0586-8, specific |
| | | for marker-exchange |
| | | plasmid |
| DVU0586-88-dnR | CGAGGCATTTCTGTCCTGGCTGG | Amplification of |
| | AGACCGTCCATCTCGTCTGC (SEQ ID | downstream region of |
| | NO: 16) | DVU0586-8 |
| DVU2481-85- | GGGAAGGCATTAACCGATACTTG (SEQ | Amplification of |
| MLD-upR | ID NO: 17) | upstream region of |
| | | DVU2481-85, specific |
| | | for marker-less |
| | | deletion plasmid |
| DVU2481-85- | CAAGTATCGGTTAATGCCTTCCC | Amplification of |
| MLD-dnF | CCGACTGGATACGCAACACC (SEQ ID | downstream region of |
| | NO: 18) | DVU2481-85, specific |
| | | for marker-less |
| | | deletion plasmid |
| DVU2809-12- | TCCTCTCCTTGTTGATGCCCTG (SEQ ID | Amplification of |
| MLD-upR | NO: 19) | upstream region of |
| | | DVU2809-12, specific |
| | | for marker-less |
| | | deletion plasmid |
| DVU2809-12- | CAGGGCATCAACAAGGAGAGGA | Amplification of |
| MLD-dnF | GGGAATGTCGTCTCACGCAG (SEQ ID | downstream region of |
| | NO: 20) | DVU2809-12, specific |
| | | for marker-less |
| | | deletion plasmid |
| DVU0586-88- | GTGACAAAGCAACGCATCTTGTG (SEQ | Amplification of |
| MLD-upR | ID NO: 21) | upstream region of |
| | | DVU0586-88, specific |
| | | for marker-less |
| | | deletion plasmid |
| DVU0586-88- | CACAAGATGCGTTGCTTTGTCAC | Amplification of |
| MLD-dnF | TCTGCCGAAGAAAGATGCCTG (SEQ ID | downstream region of |
| | NO: 22) | DVU0586-88, specific |
| | | for marker-less |
| | | deletion plasmid |
| pMO9075slic_F | CAAGGATCTGATGGCGCAGGG (SEQ ID | Amplification of |
| | NO: 23) | pMO9075 backbone |
| pMO9075slic_R | ATGGTACCTCCTGGGACTGCATTGCAG | Amplification of |
| | GGCTTCCCAACCT (SEQ ID NO: 24) | pMO0975 backbone |
| 2481_pmo_R | GATCGTGATCCCCTGCGCCATCAGATCC | Amplification of |
| | TTGTCAGGCGAAAGGACGCAGGCGCAA | DvH-FDH2 from |
| | CAA (SEQ ID NO: 25) | genomic DNA |
| 2482_pmo_F | GCAGTCCCAGGAGGTACCATATGCGAA | Amplification of |
| | TGCCTCGCAGAACGTTC (SEQ ID NO: 26) | DvH-FDH2 from |
| | | genomic DNA |
| 2482_strII_R | TCATTTTTCGAACTGCGGGTGGCTCCAA | Amplification of |
| | GCGCTGGCCTTGCGCAGGTTGACCATG | DvH-FDH2-strII |
| | AA (SEQ ID NO: 27) | |
| strII_2481_F | TGGAGCCACCCGCAGTTCGAAAAATGA | Amplification of |
| | TGGCGCGCCATCAGAAGACTTGAT (SEQ | DvH-FDH2-strII |
| | ID NO: 28) | |

Underlined regions represent overhangs necessary for assembling the fragments by SLIC.

For operon deletions, the upstream and downstream regions, respectively, included 858 bp and 806 bp (fdh1; DVU0586-0588), 795 bp and 878 bp (fdh2; DVU2485-2481), and 976 bp and 970 bp (fdh3; DVU2809-2812). Parental strain JW710²⁰⁰ was used for the deletion of fdh1 and fdh3. Confirmation by Southern blot was accomplished by digesting the genomic DNA of the parental and putative deletion strains with AgeI (NEB, Ipswich, MA), separating the DNA fragments by gel electrophoresis, and probing with the upstream region.

### Example 2: Plasmid Construction

pMO9075 backbone was amplified via Phusion polymerase (New England Biolabs #E0553S) using the primers pMo9075 slic_F and pMo9075 slic_R, separated on 0.6% TAE (BioRad QBI 351-008-131) agarose gel (BioRad 161-3102) and purified via gel extraction (Qiagen #28704). Inserts were amplified with Phusion polymerase via standard reaction conditions. Primers 2482_pmo_F and 2481_pmo_R were used to amplify FDH2 for cloning into pMO9075. Primers 2482_pmo_F and 2482_strII_R were used to amplify DVU2482, introducing the upstream vector flank to DVU2482 and a StrepII tag to the 3′-end of DVU2482. Primers strII_2481_F and 2481_pmo_R were used to amplify DVU2481 with StrepII-tag overlap (while maintaining native intergenic spacer) and downstream vector flank. Amplicons were separated in 0.6% TAE agarose gels and purified by gel extraction. Inserts were assembled with vector backbone via overlap assembly using Gibson cloning (New England Biolabs #M5510A). Assembly reactions were used to transform E. coli α-select chemically competent cells (Bioline BIO-85026) and colonies were selected on YT glucose plus 50 mg/mL spectinomycin HCl (Sigma-Aldrich S9007). For positive clones, 50 mL of transformant was grown in MDAG-11 formulated in house²⁰³ supplemented with spectinomycin, and the plasmid was purified using a Qiagen Plasmid Midi Kit (Qiagen 12943).

### Example 3: Bacterial Growth

DvH strains were grown on MOYLS4 medium (see Protocol 1 below), which was adjusted to pH 7.2 with NaOH. Thioglycolate was added after equilibration to dinitrogen (Airgas NI NF200 or research grade) and before bottling. For generating inocula, media were bottled anaerobically under dinitrogen (5 psi), 50 mL per 100 mL serum bottle (Duran Wheaton Kimble 223747) with butyl rubber stopper (Chemglass CLS-4209-14) and aluminum crimp seal (Wheaton 20-0000AS). For larger volumes, glass media bottles (Pyrex 1395500, 13951L; Fisher 06-414-1C/06-41401D) were sealed with no. 6 neoprene stoppers (RPI-259100-6) and capped with media bottle lid with a center bore to access the stopper. Bottles were autoclaved and vitamins were syringed in from a filter sterilized (RPI 256131) 1× stock just before inoculation. Protocol 1: MO medium for cultivating Desulfovibrio strains. When supplemented with yeast extract (Y), lactate (L) and sulfate (S4) it is referred to as MOYLS4. Note that DvH can also be grown in the absence of L, wherein formate (F; 60 mM) serves as the electron donor. In this case, acetate (A; 10 mM) must be included as the carbon source. Therefore, formatotrophic growth medium is referred to as MOYFAS4 (see FIG. 9).

### Materials:

Serum bottles 100 mL (Wheaton)
Media bottles 500 mL, 1000 mL, 2000 mL (Gibco or Duran); caps modified in-house to include a 7 mm center bore.
Butyl rubber stoppers (Chemglass CLS-4209-14)
Neoprene stoppers size 6 (RPI-259100-6) cut to ⅔ original height
Magnetic stirrer

### Equipment:

Autoclave
Gas manifold
pH meter

### Reagents:

| | | | |
|---|---|---|---|
| Magnesium chloride hexahydrate | Sigma-Aldrich | M9272 | BCBT8684 |
| Ammonium Chloride | Sigma | A9434-1KG | SLBS1591V |
| Calcium Chloride dihydrate | Acros | 207780010 | A014020501 |
| Tris HCl | Fisher | BP153-500 | 200711 |
| Iron (III) Chloride Hexahydrate | Acros | 423705000 | lot A013817901 |
| EDTA | Fisher | BP120-1 | lot 055880 |
| Yeast Extract- | Sigma-Aldrich | 92144-5KG-F | BCBQ9331V |
| Thioglycolate | Sigma | T0632-25g | STBH2638 |
| Sodium DL-lactate 60% syrup | Sigma | L1375-500mL | SLBR4194V |
| Sodium Sulfate | Sigma-Aldrich | 239313-2.5KG | SLBT9903 |
| Potassium phosphate dibasic | Fluka | 60353 | 1167325 |
| Sodium phosphate monobasic | Fluka | 71505 | 1203314 |
| Manganese Chloride tetrahydrate | Acros Organics | 2058950000 | A012429101 |
| Cobalt Chloride hexahydrate | Sigma | C3169 | 70K3698 |
| Zinc Chloride hexahydrate | Sigma | Z-4875 | 20K0264 |
| Sodium molybdate dehydrate | Sigma | M1003 | 085K0098 |
| Boric Acid | Sigma | B-7660 | 042K0150 |
| Nickel Chloride | ICN | 155825 | 8649C |
| Copper Chloride dehydrate | Sigma | C-6917 | 121K0014 |
| Sodium Selenate | Sigma | S8295-25G | SLBD3716V |
| Sodium Tungstate dehydrate | Aldrich | 223336-5g | MKBV3962V |
| Biotin | Sigma-Aldrich | B4501-1g | SLBS3069V |
| Folic Acid | Sigma | F7876-1g | SLBN1618V |
| Pyridoxine HCl | RPI | P50240-10.0 | 10874462 |
| Thiamine HCl | Sigma | T-4625 | 062K0103 |
| Riboflavin | Sigma | R-4500 | 072K0887 |
| Nicotinic Acid | Sigma | N-4126 | 052K0200 |
| DL Pantothenic Acid | Sigma | P-2250 | 013K0583 |
| 4-Aminobenzoic Acid | Sigma | A9878-5G | MKBZ3723V |
| Lipoic Acid | Sigma | T5625-500g | SLBS2381V |
| Choline Chloride | Fisher | AC110290500 | A0400838 |
| Vitamin B12 | Sigma | V-2876 | 112K0646 |
| Sodium hydroxide | Fisher | BP359-500 | lot 201811 |
| Sodium Resazurin | Aldrich | 199303-1G | MKBP2801V |

### Solutions

| Trace Metals Stock solution (1 L) | (g) |
|---|---|
| Manganese Chloride tetrahydrate | 0.5 |
| Cobalt chloride hexahydrate | 0.3 |
| Zinc Chloride | 0.2 |
| Sodium molybdate dihydrate | 0.05 |
| Boric Acid | 0.02 |
| Nickel chloride | 0.09 |
| Copper chloride dihydrate | 0.002 |
| Sodium selenate | 0.006 |
| Sodium tungstate dihydrate | 0.008 |

| Vitamins solution working stock (10x) | (g) |
|---|---|
| Biotin | 0.02 |
| Folic Acid | 0.02 |
| Pyridoxine HCl | 0.1 |
| Thiamine HCl | 0.05 |
| Riboflavin | 0.05 |
| Nicotinic Acid | 0.05 |
| DL Pantothenic Acid | 0.05 |
| 4-Aminobenzoic Acid | 0.05 |
| Lipoic Acid | 0.05 |
| Choline Chloride | 2 |
| Vitamin B12 | 0.01 |

pH 7.0 with KOH. Filter, sterilize and store at 20° C.

### Vitamins Solution Working Stock (1×)

10 mL of 10× filter sterilized into 90 mL of anaerobic (N₂ sparged) water. Stored at 4° C. and in the dark.

### Sodium Resazurin 0.1%

0.1 g
100 mL water

### 0.5 M EDTA

93 g EDTA
pH to 8.0 with NaOH
volume to 500 mL with MilliQ water
   FeCl/EDTA Solution 125 mM/250 mM-4.8 mL
0.162 g Iron chloride Hexahydate
2.4 mL water
2.4 mL 0.5 M EDTA

| Potassium/Sodium Phosphate 1M | per L |
|---|---|
| Potassium Phosphate dibasic | 87 g |
| Sodium phosphate monobasic | 78 g |

### MOYLS4 Medium 1 L

To ˜800 mL MiliQ water add:
1.6 g Magnesium Chloride hexahydrate
1.06 g Ammonium Chloride
0.088 g Calcium Chloride dihydrate
2 mL Potassium/Sodium Phosphate solution
6 mL Mo-Trace elements
4.73 g Tris HCl (or 15 mL 2M-pH 7.2)
1 g yeast extract
11.2 mL sodium lactate 60%
4.26 g Sodium sulfate
0.16 mL sodium resazurin 0.1%
0.48 mL Iron chloride/EDTA 125 mM/250 mM (see Methods)
Bring to 1 L and pH to 7.2 with 4M sodium hydroxide.
Bubble under N₂ gas with stirring for 1 hr
Add 0.14 g Sodium Thioglycolate

Cap bottle with number 6 neoprene stopper and medium bottle cap with center bore hole, or distribute under N₂ to serum bottles (50 mL per 100 mL bottle) and cap with butyl rubber stopper and aluminum crimp seal.

Autoclave liquid cycle. Cool to room temperature.

To finish medium, add 1× vitamin stock at 0.5 mL per 50 mL, just prior to inoculation by sterile anaerobic transfer. Add desired antibiotics at this time as well.

### Example 4: Transformation

DvH strains were grown in 50 mL MOYLS4 in 100 mL serum bottle at 37 C with nitrogen headspace to near stationary phase and chilled on ice. Cells were aerobically spun down in a 50 mL conical centrifuge tube (Corning 430828) at 7,500×g for 5 min, then washed twice in 17 mL of ice cold 15 mM Tris pH 7.2, 10% glycerol supplemented to 1 mM with dithiothreitol. The final pellet was resuspended to 1 mL in the same buffer. A 100 μL aliquot of cells was aerobically mixed on ice with 7.5 μL from plasmid midi prep (˜2-3 μg plasmid) and electroporated at 1.5 kV in an Eppendorf electroporator 2510 (1 mm gap cuvette; MBP #5510). 1 mL of sterile anaerobic MOYLS4 was immediately added and the entire volume was transferred to a bottle of MOYLS4. The bottle was incubated at 37 C (Glascol, Micro-expressoin Vertiga shaker). Once the culture recovered and became densely turbid, transfers were made to fresh medium containing 100 μg/mL spectinomycin HCl. After two rounds of growth with spectinomycin selection, freezer stocks in 10% glycerol were made. For colony selection the same medium supplemented with 1.5% agar, 5 mM cysteine, 1 mM sodium sulfide, and 100 μg/mL spectinomycin was used and kept in gas tight jars with an AnaeroGen 3.5 L Gas generating system pack (Oxoid). Colonies were picked into selective medium using a sterile 1 mL syringe (Becton Dickinson 309659) fitted with an 18-gauge needle.

### Example 5: 10 L Carboy Growth of DvH-FDH2 Producing Strain CSR21271

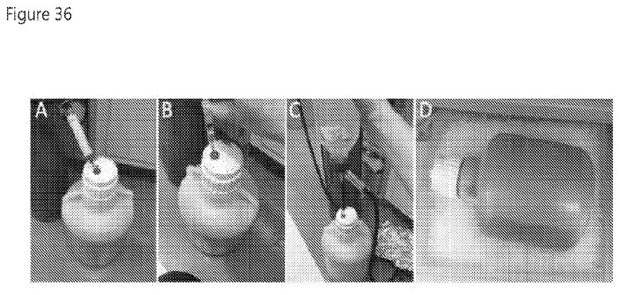

For each carboy, the strain was transferred from 10% glycerol freezer stock in MOYLS4 medium; ˜0.5 mL of stock added to a 50 mL bottle of anaerobic MOYLS4 medium, supplemented with vitamins and 100 μg/mL spectinomycin hydrochloride. Transfers were made by nitrogen purged syringe with 23-gauge needles (Becton Dickinson 305190). The culture was incubated overnight at 37 C or until mid-exponential phase of growth. 20 mL of the overnight culture was used to inoculate a 500 mL bottle of MOYLS4 medium, containing vitamins and 100 μg/mL Spectinomycin HCl. The 500 mL culture was incubated overnight at 37 C. 10 Liters of MOYLS4 medium in 2 L bottles, prewarmed, sterile, aerobic, with iron and EDTA withheld, was poured into a sterile 10 L polypropylene carboy (Thermo 2250-0020). The medium was completed by addition of filter sterilized vitamins, spectinomycin hydrochloride (Ig dissolved in 15 mL water; 100 μg/mL final) and 4.8 mL of iron chloride (125 mM; Acros 423705000)/EDTA (250 mM; Fisher BP120-1) solution. The carboy was closed and purged with nitrogen via a butyl rubber stopper port (Chemglass CLS-4209-14) affixed to the lid (FIG. 36, panel A). 5 mL of 25% sterile neutral sodium sulfide (Alfa Aesar 36622) was then injected through the port and the carboy was mixed by shaking (FIG. 36, panel B). Subsequently, the carboy was incubated at 37 C until resazurin indicator turned colorless. The 500 mL culture (OD₅₅₀ ˜0.6) was then transferred into the carboy via sterile rubber transfer line (VWR-62993-726), 18-gauge needles (Becton Dickinson 305196) and under nitrogen pressure (FIG. 36, panel C). The carboy was placed in an incubator (Sanyo MCO-17A1C) at 37 C and the optical density (OD) was monitored at 550 nm (Beckman DU-800 spectrophotometer) via 1 mL samples removed from the same port. Once OD₅₅₀ nm plateaued, the carboy was chilled in the cold room overnight (FIG. 36, panel D) and then harvested by centrifugation at 8,000×g (Beckman Avanti HP-26 XPI) in 1 L bottles. Cell pellets were transferred to 50 mL conical centrifuge tubes, respun (7,500×g; Corning 430828), and then froze at −80 C.

### Example 6: Protein Expression and Purification

Strep-tag II-tagged DvH-FDH2 was purified from strain CSR21271 (see Table 6). Unless specified otherwise all the following steps were done at 4 C and under atmospheric conditions. Nitrate, azide, or thiols were not used at any step of the purification or storage. Cells (˜18 g) were suspended in six volumes of 50 mM sodium phosphate (Fluka 71505, Sigma-Aldrich S0786), pH 7.4, containing 150 mM sodium chloride and 1 mL of 50×Complete Proteinase inhibitor (Roche 45582400; 1 tablet in 1 mL of MilliQ water), by gentle pipetting in cold buffer. Cells were lysed using an Avestin C3 homogenizer and cell debris spun down at 4500×g (Beckman Avanti HP-26 XPI) for 15 min. Membrane vesicles were removed by centrifugation at 285,000×g 1 hr (Beckman Optima L100XP). The clarified lysate was then fractionated by ammonium sulfate precipitation with fractions pelleted at 10,000×g for 10 min and the 40-70% saturating fraction was retained and exchanged via centrifugal concentrator (Amicon 30 kDA molecular weight cutoff) into 100 mM Tris-HCL buffer pH 8, containing 150 mM NaCl and 1 mM EDTA. The sample was loaded on to streptactin-XT superflow resin (IBA-LifeSciences) and the column was washed with 40 volumes of the same buffer. StrepII-tagged protein was eluted by several column volumes of 100 mM Tris-HCl buffer, pH 8, containing 150 mM NaCl, 1 mM EDTA, and 50 mM biotin (IBA-LifeSciences 2-1016-005). The protein was concentrated via centrifugal concentrator (Amicon 30 kDA MWCO) and exchanged into 20 mM Tris-HCl buffer pH 8.0, with or without 10% glycerol (Sigma-Aldrich 49770) and stored at −80 C for future use. The protein concentration was estimated by BCA assay (Thermo Fisher) versus a BSA standard.

### Example 7: Gel Electrophoresis

DvH-FDH2 was separated on a Nupage 4-12% Bis-Tris Gel (Thermo Fisher). The running buffer was 1×MES-SDS. The sample was loaded as 5 μL of 12 μM DvH-FDH2 in 62.5 mM Tris-HCl buffer, containing 1.5% SDS, 10% sucrose, 0.0075% bromphenol blue, pre-incubated at room temp (23 C) for 30 minutes and then heated 5 minutes at 50 C. The protein was run alongside Precision plus Kaleidoscope prestained standards (Bio-Rad #1610375) for 100 minutes at 100 Volts (Invitrogen mini gel talk A25977). The gel was fixed in 40% methanol, 10% acetic acid, stained in 30% methanol, 10% acetic acid, and 0.05% Coomassie blue G-250, and destained in 8% acetic acid. Gels were scanned with a gel doc imager (Bio-Rad).

### Example 8: Chromogenic Visualization (In-Gel Assay)

DvH-FDH2 was separated on a standard Tris buffered 5% polyacrylamide gel, 2.6% crosslinker gel supplemented with 0.05% triton X-100 (Fisher BP151-100). The running buffer was 25 mM Tris, 192 mM glycine and 0.05% triton X-100 (v/v). Every other lane was loaded with 7.5 μL of 3 μM FDH2 in 20% sucrose, 0.25M Tris pH 6.9, 0.05% triton, 0.0125% bromphenol blue. Electrophoresis was conducted at 100 V for 209 minutes, 10 mA limit at 4 C. Lanes were cut into strips then assayed in 10 mL of 0.24 mg/mL nitroblue tetrazolium (NBT; Invitrogen N6495) in 20 mM Tris pH 8.0 with or without 10 mM formate (added as 100 μL of 1 M). Strips were incubated with shaking for 15 minutes and then washed with MilliQ water for 3×5 min, and then scanned on a Bio-Rad Gel Doc imager.

### Example 9: NBT Strip Assay

3 mm strips of chromatography paper (Whatman), cut with a paper cutter, were soaked with a solution of 300 μM NBT (1 mL of 3 mM in water), 30 μM PMS (Sigma P9625) (1 mL of 300 μM in ethanol) in 8 mL Tris-buffered saline pH 7.5, with or without 10 mM formate (Aldrich 798630-500 g) (100 μL of 1 M in water). Strips were then spotted with 5 μL of 12 μM enzyme or control solution (buffer only) and monitored for color development. PMS was added to the NBT solutions to 30 μM final from a 30 mM stock in water before soaking the strips. For the NBT only experiment, strips of Whatman chromatography paper (˜8.8 cm²) were soaked with 140 μL of 293 μM NBT in 20 mM Tris pH 8.0 with or without 10 mM sodium formate. FDH2 was spotted onto strips as a 5 μL drop of 12 μM of enzyme in 20 mM Tris pH 8.0.

### Example 10: Metal Analysis

Inductively coupled plasma mass spectrometry (ICP-MS; iCAP-RQ Thermo Scientific) was used in the KED mode to assess the metal stoichiometry of DvH-FDH2. Protein samples were prepared by vortexing each protein sample for 10 seconds followed by centrifugation at 100×g for 20 seconds. 25 L of each sample were put into 15 mL conical tubes followed by the addition of 200 μL of Optima grade HNO₃. Samples were digested for 20 minutes at room temperature followed by the addition of 9.775 mL of Millipore H₂O for a final acid matrix of 2% HNO₃ (v/v). QCS27 was used as a multi-element standard as well as W individual standard. The following isotopes were chosen for analysis: ^{56,57}Fe, ^{63,65}Cu, ^{77,78,82}Se, ^{95,96,98}Mo, and ^{182,183,184}W. The internal standards selected for analysis were: ⁶Li, ⁴⁵Sc, ⁸⁹Y ¹¹⁵In, ²⁰⁹Bi. All sample were run with one survey run and three main peak jumping runs.

Enzymology. Protein Concentration Assessment. Because TN (k_{cat}) calculation requires that the enzyme concentrations be determined accurately, four approaches were utilized (a) quantification via known extinction coefficients (F) of [4Fe-4S] clusters with ε₄₁₀=15,000 M⁻¹ cm⁻¹ per [4Fe-4S]²⁺ cluster¹⁸¹ or ε₄₁₀=4000 M⁻¹ cm⁻¹ per Fe atom;⁸¹ (b) evaluating the extinction coefficient of DvH-FDH2 via electronic spectroscopy; (c) bicinchoninic acid assay (Thermo Fisher) versus a BSA standard; and (d) cross-checking values with ε₄₁₀=43,446M⁻¹ cm⁻¹ reported for DvH-FDH1.²³ Using these strategies, the final concentration of DvH-FDH2 used in all enzyme kinetics and O₂ reduction measurements was estimated to be in the range of 1.1-1.6 nM. Since significant systematic errors are associated with enzyme concentration determination via dyebinding or electronic spectroscopy, the results are typically calibrated via quantitative amino acid analysis.¹⁸² Furthermore, cluster concentrations would be independently assessed by iron¹⁸³ and sulfide¹⁸⁴ analysis. These corrections could not be made here. Unless specified otherwise, the highest protein concentration value (1.6 nM) was used to derive kinetic parameters.

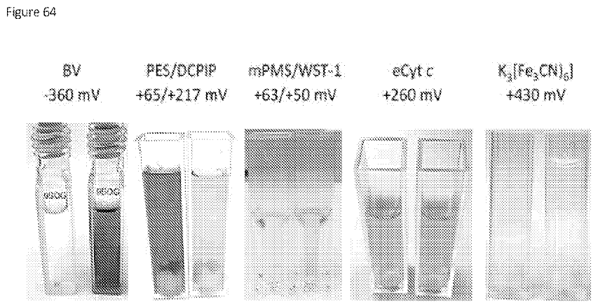

Evaluation of Electron Acceptor Specificity. BV, PMS, 1-methoxy-phenazinemethosulfate (mPMS; Dojindo M003-10), PES, DCPIP, water-soluble tetrazolium-1 (WST-1; Fisher/Dojindo NC1343907), potassium ferricyanide (FC; Sigma 244023), and O₂ were tested for their ability to accept electrons derived from enzymatic formate oxidation. Reaction conditions are described in the legends to FIG. 64. See also video at pubs.acs.org/doi/suppl/10.1021/acscatal.2c00316/suppl_file/cs2c00316_si_002.mp4 or ndownloader.figstatic.com/files/36617584.

### Example 11: Benzyl Viologen Assay

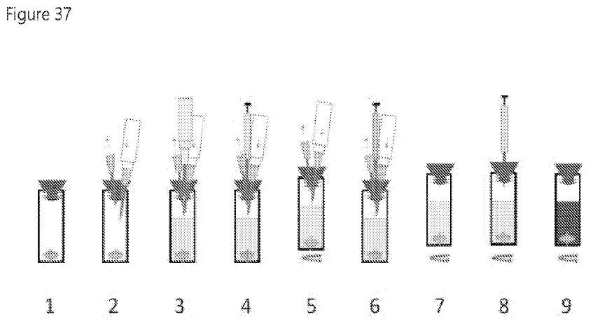

In this assay, the one-electron reduction of BV (colorless) by DvH-FDH2 produces BV⁺ (blue or purple), which is followed spectrophotometrically.^{28,185} The workflow is described in FIG. 37. FDH2 was assayed in an argon (Airgas AR-300) atmosphere with 2 mM or 20 mM BV (Alfa Aesar H66836) in 50 mM Tris pH 8.0 with 20 mM glucose and supplemented with GO (Sigma-G7141-50ku) 1 U/mL, and catalase (Sigma C1345-G) 1 g/mL, in a stoppered quartz cuvette (Helma 110-10-40) fitted with a Suba-Seal silicone septum (Sigma Z279730-25ea) and an 8 mm×3 mm pivoted spin bar (VWR 37119-6183). Assays were performed under argon in a UV-2600i spectrophotometer (Shimadzu Life Sciences) equipped with a T2 Peltier/stirring unit (Quantum; T=25° C.; stirring speed 650 rpm) and monitored at 557 nm. Measurements were started by the addition of 10 μL formate or buffer blank using a 10 μL Hamilton syringe (under an argon headspace) to a 2.5 mL reaction mix containing pre-equilibrated 1.6 nM FDH2.

### Example 12: Phenazine Ethosulfate (PES)/Dichlorophenolindophenol (DCPIP) Assay

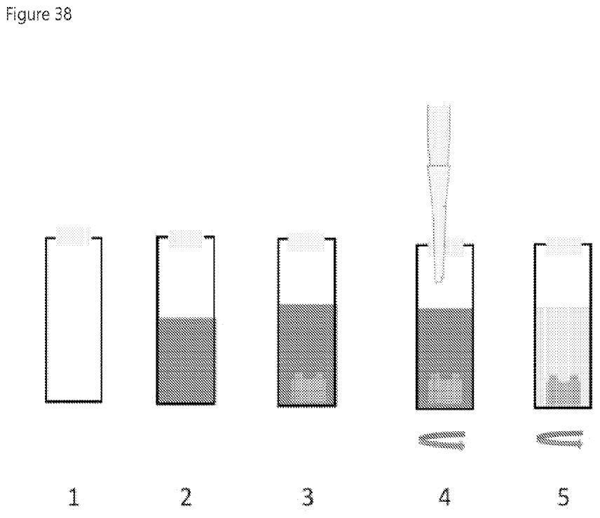

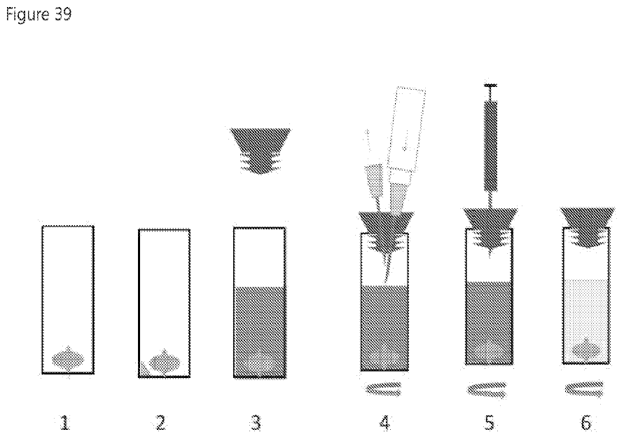

In this dye-linked assay, PES (E_{m,7}=+65 mV) serves as the primary electron acceptor. Following its reduction by FDH2, PES nonenzymatically transfers electrons to a second dye, DCPIP, facilitating spectrophotometric detection.⁹¹ Workflows are described in FIGS. 38 and 39. PES was chosen over PMS because of its long-term stability.¹²⁰ Furthermore, necessary precautions were taken in handling and preparing the reagents, including protecting the reagents from light.¹⁸⁷ DvH-FDH2 was assayed in 1 mM PES (Sigma-Aldrich P4544-1G)/93 μM DCPIP (98% pure; Acros 152870100) in 50 mM Tris pH 8.0. Reactions, 2.5 mL volume, were set up to the desired formate concentration and then started by addition of 10 μL of 400 nM FDH2 (1.6 nM final). Precipitation of reduced PES precluded used of higher concentrations of the electron acceptor. Therefore, 1 mM PES was used. Assays were performed using a UV-2600i spectrophotometer (Shimadzu Life Sciences) equipped with T2 Peltier/stirrer accessory (Quantum) and monitored at 600 nm. For aerobic measurements (FIG. 38), reactions were performed in open top styrene disposable cuvettes (Brand 75907D) with 6 mm×9 mm cuvette stirrer (Cowie 001.1609) spun at 400 rpm, and reactions started by addition of 10 μL of 400 nM FDH2 via micropipette. Some aerobic experiments were conducted using a Cary 300 UV-Vis spectrophotometer (Agilent) at a volume of 3.0 mL with api 9 mm×8 mm spin bar (Sigma Z363545) cuvette stirrer. For the anaerobic conditions (FIG. 39), reactions were set up under argon in screw cap quartz cuvettes (Starna 1-SOG-10_GL14-S) sealed with Suba-Seal 13 white rubber septa (Sigma Z167258). The stirrer was an 8 mm×3 mm pivot bar (VWR 37119-6183), stirring at 650 rpm. Reactions were started by addition of FDH2 via 10 μL Hamilton syringe (701N 80300) under an argon atmosphere.

### Example 13: Data Analysis

For classical steady-state kinetics, initial velocities (guided by residual plots) were obtained using ICEKAT.¹⁸⁸ KinTek Explorer^{84,89} (version 10.1.6, KinTek Corporation) was used to perform global fitting of enzyme kinetics data to Schemes 1 and S1. This is based on numerical integration of rate equations. Confidence contour analysis was carried out to assess whether the parameters were properly constrained by the data.

### Example 14: Spectroscopy. Electronic Data Analysis

FDH2 spectra were collected at 23 C in 50 mM Tris pH 8.0 using a screw cap 1 cm pathlength quartz cuvette (Starna; 1-SOG_10_GL14s with GL14S cap). For aerobic spectra the spectrum of air equilibrated enzyme was collected, formate was added to 10 mM and the formate reduced spectrum was collected. The sample was then capped with silicone septa (Starna GL14/SI) and 10 μL of 2 mM of dithionite was added under argon before collecting a spectrum. For anaerobic measurements, FDH2 was gassed with argon in the sealed cuvette before addition of formate or dithionite. Reduced spectra were also measured using dithionite as the sole reductant (in the absence of formate). Dithionite was prepared in an anaerobic buffer immediately before use.

### Example 15: Electron Paramagnetic Resonance (EPR)

All samples were prepared in 20 mM Tris-HCl, pH 7.6 containing 10% glycerol (v/v). Anaerobic samples were first purged with Ar and then transferred to septum-sealed, Ar-flushed EPR tubes and reduced with either 20 mM anaerobic sodium formate or ˜4 mM anaerobic sodium dithionite. Aerobic samples were reduced directly in open EPR tubes with either 20 mM sodium formate or ˜2 mM sodium dithionite. All samples were subsequently frozen in a dry ice/ethanol bath, then transferred to liquid nitrogen for storage. The anaerobic sample reduced with sodium dithionite was incubated for 12 h prior to freezing.

EPR spectra were recorded using a Brüker EMX spectrometer operating WinEPR version 4.33 acquisition software and equipped with a Bruker ER 4119HS high sensitivity X-band cavity and gaussmeter. Temperature was controlled with a Brüker variable temperature unit and a liquid nitrogen or liquid helium cryostat. For purposes of comparison, all spectra were calibrated to a microwave frequency of 9.385 GHz. Integration of the iron-sulfur EPR signals was performed using spectra collected at 15 K, using Megasphaera elsdenii ferredoxin (product of locus AL 641500; UniProtKB-P00201) as a standard. Detailed instrument settings are included in the figure captions. Simulations were performed using the EasySpin 4.5.5 software package.¹⁸⁹ Simulations included a “weight” term, which was used to estimate the relative contribution of each component to the composite spectrum.

### Example 16: Nuclear Magnetic Resonance

NMR data were recorded on an Agilent DD2 500 MHz spectrometer equipped with a 5 mm quadruple (¹H, ¹³C, ¹⁵N, ³¹P) PFG Penta Probe, which was maintained at 25 C. ¹³C data were acquired with 70332 points with a spectral width of 30,478 Hz, 242 ppm centered at 110 ppm, with proton-decoupling on throughout the experiment (1 s delay between transients and 1.15 s of acquisition time) and the number of transients collected ranged from 64 to 1024. The fids were zero-filled and multiplied with a 3-Hz line-broadening function prior to Fourier-transformation; the final size of the spectrum was 65536 points. Proton data were recorded with 16384 points with a spectral width of 7530 Hz (15 ppm centered at 4.7 ppm) with pre-saturation (2 s) to suppress the water peak; 1 s delay between transients were used. Additional parameters are detailed in the supplement. ¹³C-formate (9.5-10.5 mM in 100 mM sodium phosphate buffer, pH 6 or 7.5) and ¹³C-sodium bicarbonate (4.8 mM in 100 mM sodium phosphate buffer, pH 6) reference spectra were first collected using standard 5 mm thin-walled NMR tubes (Wilmad). 10% D₂O was used to obtain internal signal lock. Subsequently, 1.3 μM of DvH-FDH2 was added to the tube containing ¹³C-formate (pH 7.5), mixed, and spectra were recollected. Upon completion, 2 mM PES was added to the same tube, mixed, and remeasured. Independently, this process was repeated with ¹³C-formate at pH 6. NMR data were processed with MestReNova NMR suite version 14.2.1-27684.

### Example 17: Quantification of O₂ Reduction; Oxygen Consumption

A Clark-type O₂ electrode (Hansatech Instruments Oxygraph⁺System) was used to measure O₂ uptake at 23 C. The electrode was calibrated with dithionite. Order of reagent additions are described in the respective figure legends. A Clark-type O₂ electrode (Oxygraph Plus System from Hansatech Instruments, UK) was used to monitor changes in the dissolved O₂ concentration, which corresponds to 267 μM at 23° C. O₂ saturation under these conditions would be equivalent to 1.27 mM. A decrease in the O₂ level would indicate that O₂ was being consumed during aerobic catalysis. Conversely, O₂ evolution would be diagnostic of catalase activity. The electrode was calibrated each time before use with air-saturated water and dithionite as per the manufacturer's instructions. Freshly made reagent stocks and buffer solutions were used throughout. 1 mL reactions were performed at 23° C. in a closed cell using air-saturated 100 mM Tris-HCl, pH 8, containing 1 mM EDTA (Fisher BP120-1). The latter was added to limit adventitious metal ions from mediating O₂ consumption. After obtaining a stable baseline with the buffer, 10 mM formate was added, and the baseline was allowed to stabilize. The reaction was started by the addition of 50 nM DvH-FDH2. Once the O₂ consumption plateaued, 2 μM catalase (Sigma C1345-G) was added. Catalase catalyzes the redox disproportionation of H₂O₂ to water and dioxygen (2H₂O₂→2H₂O+O₂). To test the effect of additives, the order of addition was changed. For example, to test whether DvH-FDH2 had catalase activity, H₂O₂ was added to the buffer first, followed by the enzyme. Similarly, to assess the effect of superoxide dismutase (SOD) on O₂ uptake, SOD (Sigma S5395-15KU; 250 U/mL) was the first component to be added. SOD catalyzes the dismutation of superoxide radical anion: O₂^{·-}+2H⁺→2H₂O₂+O₂. O₂ consumption rates were calculated as described before.¹⁹⁰ Initial velocities were determined from the slopes of [O₂] versus time traces after subtracting O₂ consumption under the same experimental conditions without FDH2.

### Example 18: Quantification of O2 Reduction; H₂O₂ Production

Amplex Red (AR) Method. 5 mg of AR (Invitrogen A12222) was dissolved in 0.9725 mL of neat dimethyl sulfoxide (DMSO) to yield a 20 mM solution. Horseradish peroxidase (HRP) (Sigma P8250-5ku) was prepared at a concentration of 10 U/mL (45.5 g/mL) in sodium phosphate pH 7.4. Prior to the assay, a 2× working solution was prepared from 10.6 μL of 20 mM AR, 80 μL of HRP, 1.6 μL of 0.5 M diethylenetriaminepentaacetic acid (DTPA; TCI D0504), and 3.9 mL of 50 mM sodium phosphate pH 7.4 and kept in the dark. Production of H₂O₂ was measured by preparing reaction mixtures in a Costar 3915 black flatbottom 96-well plate. Reactions used 50 mM sodium phosphate pH 7.4, with desired amounts of sodium formate added from a 50 μM stock and initiated by addition of 5 μL of 32 nM DvH-FDH2 in the same buffer to a volume of 50 L. This approach allowed H₂O₂ generation to commence prior to the introduction of the AR/HRP mixture. A H₂O₂ (Sigma-Aldrich H1009-100 mL) standard curve was generated in the same buffer to a volume of 50 μL. Detection was initiated by addition of 50 μL of the 2×AR/HRP working solution, and fluorescence was scanned in top read mode at medium sensitivity on a SpectraMax M2 (Molecular Devices) plate reader (excitation 530 nm and emission 590 nm) every 4 min for 12 min (23° C.). Independently, it was assessed whether outcomes differed when the order of addition was varied. Therefore, in one set of assays, 5 μL of 32 nM FDH2 was added after AR/HRP. Here, 0.5 mM DTPA was used instead of 0.1 mM.

Coumarin Boronic Acid (CBA) Assay. 10 mg of coumarin boronic acid (CBA) Cayman Chemicals 14051) was dissolved in 3.33 mL of DMSO. 101 μL of the CBA stock and 1.6 μL 0.5MDTPA were added to 3.9 mL 50 mM sodium phosphate pH 7.4 to produce a 2× working solution. The remaining steps essential identical to those used in the AR assay except that the plate was shaken at 400 rpm in an incubator (23° C.) for 15 min prior to CBA addition. Fluorescence detection was initiated by addition of 50 μL of the CBA 2× working solution, and the plate was scanned in fluorescence mode (excitation 332 nm and emission 470 nm). This method was also used in the context of redox bifurcation to quantify H₂O₂ production by DvH-FDH2 in the presence of 30 μM cytochrome c. Independently, the latter was incubated with H₂O₂ for the same duration (in the absence of DvH-FDH2) to assess the extent of peroxidase activity. Cytochrome c concentration was estimated as follows: oxidized ε₄₁₀=106.1 mM⁻¹ cm⁻¹ and reduced ε₅₅₀=29.5 mM⁻¹ cm⁻¹.

The following controls were common to both CBA and AR assays. (1) Heat-denatured DvH-FDH2, (2) addition of catalase (100 U/mL), (3) addition of SOD (10 U/mL), (4) FDH2 omitted, (5) fluorogenic substrate excluded, (6) formate excluded, and (7) buffer only.

### Example 19: Quantification of O₂ Reduction; Superoxide Generation

Native (Sigma C2506) and partially acetylated (Sigma C4186) equine heart cytochrome were used to assess superoxide production by DvH-FDH2. The integrity of oxidized cytochrome c was validated by establishing the presence of a 695 nm transition. The reduction of 30 μM (native) or 60 μM (partially acetylated) cytochrome c by DvH-FDH2 was followed at 550 nm in a 1 cm pathlength cell (Shimadzu UV-2600i spectrophotometer). A 2-fold higher concentration of acetylated cytochrome c was used to offset its slightly weaker reactivity with superoxide.¹⁰⁴ The reaction mix (total volume 2.5 mL) was stirred (Cowie 001.1609) at 300 rpm (Quantum T2/Peltier unit) and maintained at 25° C. For aerobic experiments, open-top styrene disposable cuvettes (Brand 75907D) were used. For anaerobic measurements under argon, screw-capped quartz cuvettes (Starna 1-SOG-10_GL14-S) sealed with Suba-Seal 13 white rubber septa (Sigma Z167258) were used. To 50 mM Tris-HCl buffer, pH 8, containing cytochrome c, FDH2 (1.6 nM final) was added first to obtain the background signal. Subsequently, 10 μM formate was added to start the reaction. Upon completion, ˜2 mM dithionite was spiked into the mix to estimate the amount of remaining oxidized protein. Controls devoid of formate, enzyme, and cytochrome c were also employed. The effect of SOD (10-100 U/mL) or catalase (100-400 U/mL) was tested independently.

Example 20. Structural Analysis. Protein alignments were constructed using MUSCLE or MAFFT. Structural alignments were performed using Chimera v1.16. Amino acid sequences of the large (DVU2482) and small (DVU2481) subunits of DvHFDH2 were input together for running structure predictions using a modified version of AlphaFold2.1.¹⁵³ Because this algorithm does not recognize Sec, a Cys was substituted and Tat signal peptide (see FIG. 11A) was not included. A dedicated Google Colab notebook [AlphaFold.ipynb-Colaboratory (google.com)], which does not utilize homologous structures for making predictions was used with default settings. The structures of DVU2482 and DVU2481 were also predicted using a full version of AlphaFold2.1. The resulting heterodimeric structures were superposed on the DvH-FDH1. counterpart determined via X-ray crystallography (PDB ID: 6SDV²⁰) to assess similarities and differences. Difference distance matrices were computed using Chimera v1.16. Structure visualizations and manipulations were done via PyMOL.

### Example 21: FDH Electrodes

Unless stated specifically, all chemicals were purchased from Sigma Aldrich. Highly oriented pyrolytic graphite (HOPG) were obtained from Bruker AFM Probes. Multi-walled carbon nanotubes (MWCNTs) were purchased from Cheap Tubes (catalog number 030303). Ethylene glycol diglycidyl ether (EGDGE) was obtained from Polysciences, Inc. Known synthesis of benzyl viologen modified linear polyethyleneimine (BV-LPEI), naphoquinone modified linear polyethyleneimine (NQ-LPEI) and cobaltocene modified poly allyl amine (Cc-PAA) was used²²⁴⁻²²⁶.

The electrodes were prepared as follows. Enzyme preparation: DvH-FDH2 shipped frozen on dry ice in a buffer containing 50% glycerol was first solvent exchanged via centrifugation to remove the glycerol. The buffer used for this purpose was 50 mM Tris-HCl buffer, pH 8. This resulted in a final enzyme concentration of approximately 10 mg/mL. The enzyme sample was prepared immediately before being used for immobilization. Preparation of Multi-walled carbon nanotube (MWCNT) suspension: COOH-functionalized MWCNTs were added to 100% isopropanol at a final concentration of 5 mg/mL. The resulting suspension was disrupted by sonication for 1 h. The final suspension was then left to stand for an additional hour prior to use. Stored at room temperature (methodology is identical to that reported in Milton 2017¹⁹⁷).

HOPG was cut into 5 mm×12 mm×1 mm. No other modification was done. The HOPG electrode is made up of graphite layers. Therefore, washing is not required. Instead, Scotch tape was used to peel off the layer, resulting in a clean surface. 10 μL of 5 mg mL-1 MWCNTs dispersion in isopropanol was deposited on the clean electrode surface and the isopropanol was allowed to dry in air. This happened in less than 3 minutes. After this step, the MWCNTs were adsorbed to the electrode surface. The incubation time did not make a difference in the adsorption process. All the deposition was done on the largest square surface (12 mm*12 mm).

Electrochemistry buffers and solutions: Freshly prepared 50 mM Tris-HCl buffer, pH 8.0. A formate solution (1 M) was prepared using this 50 mM Tris-HCl buffer.

Electrochemistry equipment: Bioelectrodes were initially evaluated using cyclic voltammetry with a potentiostat operating in a standard 3-electrode half-cell configuration. Typically, a large platinum counter electrode was used along with common reference electrode (saturated calomel electrode). Methodology identical to that used in Milton 2017¹⁹⁷.

Using a positive displacement pipette, 10 μL of the MWCNTs suspension was first deposited onto the surface of HOPG to yield an approximate loading of 0.33 mg/cm2. Subsequently, once the electrode was dry (3-5 min at room temperature), 5 μL of the enzyme solution was placed onto the modified surface (MWCNTs, using electrode area of 0.15 cm2) of the HOPG and left to dry for 90 mins. The resulting electrode was neither rinsed nor washed before use. Initially, bioelectrodes were electrochemically cycled for 10 complete cycles at 50 mV/s before the “real” measurement. This allowed for removal of any unbound enzyme on the electrode. During electrocatalytic measurements, the electrolyte solutions were not stirred.

As to the Laccase cathode, anthracene-modified MWCNTs were added to 150 L of laccase solution (20 mg ml⁻¹ in pH 6.5 citrate/phosphate buffer, 0.2 M). The resulting mixture was sonicated for 10 mins and then vortexed. 50 μL TBAB-Nafion solution was added and one more sonication/vortex was performed. This mixture was evenly painted on 3 Toray paper electrodes (0.8 cm2).

As to the FDH anode modified with redox polymers, 21 μL of BV-LPEI or NQ-LPEI (10 mg/mL), 9 μL of FDH (10 mg/mL) and 1.125 μL of EGDGE (10% in water) were mixed and then vortexed. 10 μL of the mixture was deposited on the Toray paper electrode (0.25 cm²) and dried for 3 hours. 21 μL of Cc-PAA (5 mg/ml), 9 μL of FDH (10 mg/mL) and 1.125 μL of EGDGE (3% in water) were mixed and then vortexed. 10 μL of the mixture was deposited on the Toray paper electrode (0.25 cm2) and dried for 3 hours.

To test these electrodes, solution-based electrochemistry was utilized, in which 0.12 mg/mL FDH solution (50 mM Tris-HCl buffer, pH 8.0) was tested with 150 μM mediator.

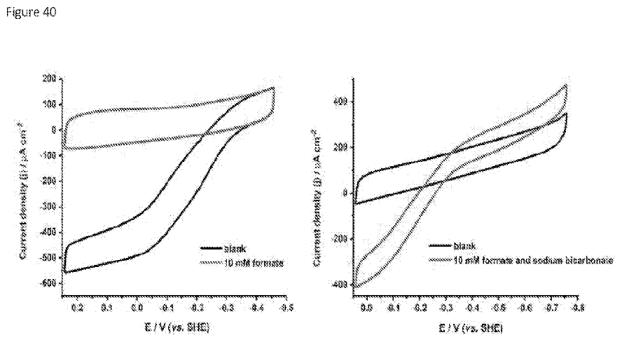

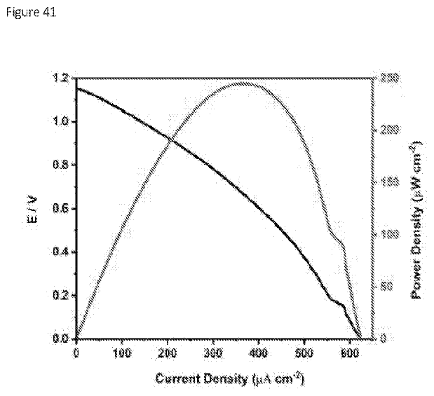

The results are shown in FIGS. 40-48. Specifically, FIG. 40 shows cyclic voltammetry charts (CVs) for formate oxidation and CO₂ reduction in the case of FDH adsorption on HOPG. FIG. 41 shows fuel cells with a laccase cathode, in the case of FDH adsorption on HOPG.

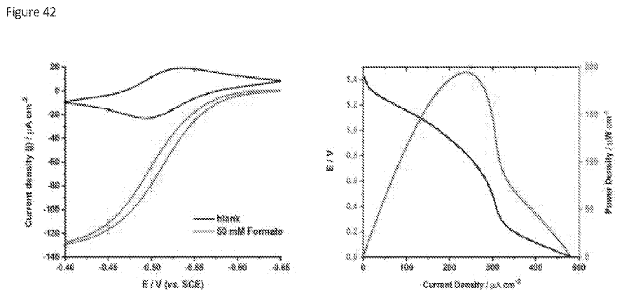

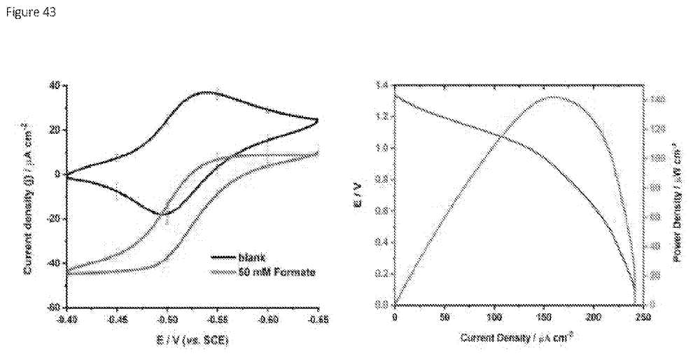

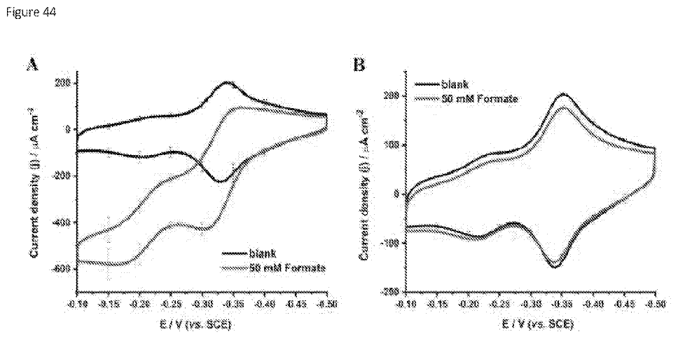

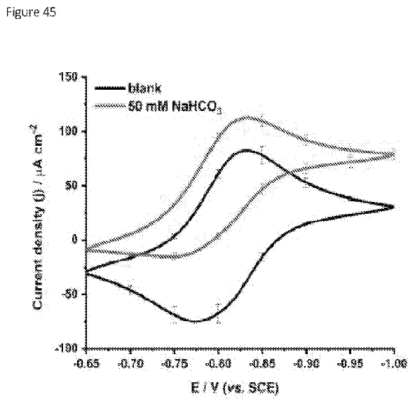

FIG. 42 shows CVs and polarization and power curves in the case of FDH immobilized with a redox polymer (BV-LPEI) under anaerobic conditions. FIG. 43 shows CVs and polarization and power curves in the case of FDH immobilized with a redox polymer (BV-LPEI) also under anaerobic conditions, but tested in open air. FIG. 44 shows CVs in the case of FDH immobilized with a redox polymer (NQ-LPEI) under anaerobic conditions. FIG. 45 shows CVs in the case of FDH immobilized with a redox polymer (Cc-PAA) under anaerobic conditions.

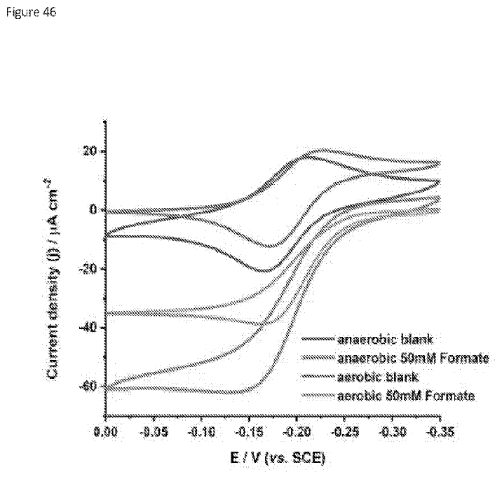

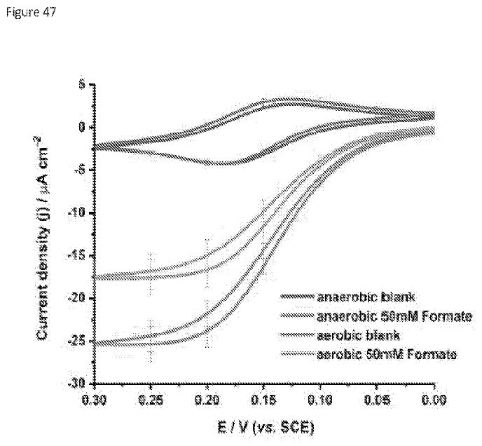

FIGS. 46 and 47 illustrate CVs of FDH in solution alone, and in combination with ferrocenium hexafluorophosphate, respectively.

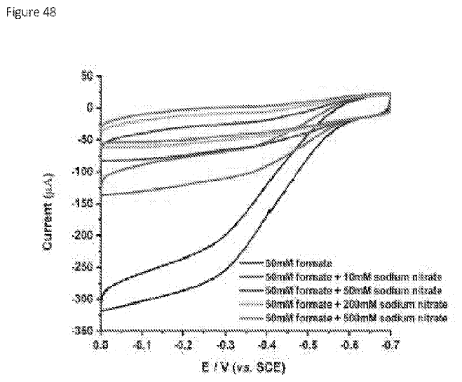

Finally, FIG. 48 illustrates the CV of FDH inhibition, where FDH/HOPG was immobilized under aerobic conditions but tested anaerobically.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
(1) Pinske, C.; Sawers, R. G. Anaerobic Formate and Hydrogen Metabolism. EcoSal Plus 2016, 7. DOI: 10.1128/ecosalplus.ESP-0011-2016
(2) Hughes, E. R.; Winter, M. G.; Duerkop, B. A.; Spiga, L.; Furtado de Carvalho, T.; Zhu, W.; Gillis, C. C.; Buttner, L.; Smoot, M. P.; Behrendt, C. L.; Cherry, S.; Santos, R. L.; Hooper, L. V.; Winter, S. E. Microbial Respiration and Formate Oxidation as Metabolic Signatures of Inflammation-Associated Dysbiosis. Cell Host Microbe 2017, 21, 208-219.
(3) Ternes, D.; Tsenkova, M.; Pozdeev, V. I.; Meyers, M.; Koncina, E.; Atatri, S.; Schmitz, M.; Karta, J.; Schmoetten, M.; Heinken, A.; Rodriguez, F.; Delbrouck, C.; Gaigneaux, A.; Ginolhac, A.; Nguyen, T. T. D.; Grandmougin, L.; Frachet-Bour, A.; Martin-Gallausiaux, C.; Pacheco, M.; Neuberger-Castillo, L.; Miranda, P.; Zuegel, N.; Ferrand, J. Y.; Gantenbein, M.; Sauter, T.; Slade, D. J.; Thiele, I.; Meiser, J.; Haan, S.; Wilmes, P.; Letellier, E. The gut microbial metabolite formate exacerbates colorectal cancer progression. Nat. Metab. 2022, 4, 458-475.
(4) FEEDAP. Scientific opinion on the safety and efficacy of formic acid when used as a technological additive for all animal species. EFSA J. 2014, 12, 3827.
(5) Yishai, O.; Lindner, S. N.; Gonzalez de la Cruz, J.; Tenenboim, H.; Bar-Even, A. The formate bio-economy. Curr. Opin. Chem. Biol. 2016, 35, 1-9.
(6) Du, D.; Lan, R.; Humphreys, J.; Tao, S. Progress in inorganic cathode catalysts for electrochemical conversion of carbon dioxide into formate or formic acid. J. Appl. Electrochem. 2017, 47, 661-678.
(7) Kar, S.; Rauch, M.; Leitus, G.; Ben-David, Y.; Milstein, D. Highly efficient additive-free dehydrogenation of neat formic acid. Nat. Catal. 2021, 4, 193-201.
(8) Roden, E. E.; Jin, Q. Thermodynamics of microbial growth coupled to metabolism of glucose, ethanol, short-chain organic acids, and hydrogen. Appl. Environ. Microbiol. 2011, 77, 1907-1909.
(9) Windman, T.; Zolotova, N.; Schwandner, F.; Shock, E. L. Formate as an energy source for microbial metabolism in chemosynthetic zones of hydrothermal ecosystems. Astrobiology 2007, 7, 873-890.
(10) Amenabar, M. J.; Colman, D. R.; Poudel, S.; Roden, E. E.; Boyd, E. S. Electron acceptor availability alters carbon and energy metabolism in a thermoacidophile. Environ. Microbiol. 2018, 20, 2523-2537.
(11) Crable, B. R.; Plugge, C. M.; McInerney, M. J.; Stams, A. J. Formate formation and formate conversion in biological fuels production. Enzyme Res. 2011, 2011, 532-536.
(12) Sawers, G. The hydrogenases and formate dehydrogenases of Escherichia coli. Antonie van Leeuwenhoek 1994, 66, 57-88.
(13) Maia, L. B.; Moura, I.; Moura, J. J. G. Carbon Dioxide Utilisation—The Formate Route. In Enzymes for Solving Humankind's Problems: Natural and Artificial Systems in Health, Agriculture, Environment, and Energy; Moura, J. J. G., Moura, I., Maia, L. B., Eds.; Springer Nature: Switzerland, 2021; pp 29-81.
(14) Niks, D.; Hille, R. Molybdenum- and tungsten-containing formate dehydrogenases and formylmethanofuran dehydrogenases: Structure, mechanism, and cofactor insertion. Protein Sci. 2019, 28, 111-122.
(15) Boyington, J. C.; Gladyshev, V. N.; Khangulov, S. V.; Stadtman, T. C.; Sun, P. D. Crystal structure of formate dehydrogenase H: catalysis involving Mo, molybdopterin, selenocysteine, and an Fe₄S₄ cluster. Science 1997, 275, 1305-1308.
(16) Niks, D.; Hille, R. Reductive activation of CO₂ by formate dehydrogenases. Methods Enzymol. 2018, 613, 277-295.
(17) Sebban, C.; Blanchard, L.; Bruschi, M.; Guerlesquin, F. Purification and characterization of the formate dehydrogenase from Desulfovibrio vulgaris Hildenborough. FEMS Microbiol. Lett. 1995, 133, 143-149.
(18) ElAntak, L.; Dolla, A.; Durand, M.-C.; Bianco, P.; Guerlesquin, F. Role of the tetrahemic subunit in Desulfovibrio vulgaris hildenborough formate dehydrogenase. Biochemistry 2005, 44, 14828-14834.
(19) Costa, C.; Teixeira, M.; LeGall, J.; Moura, J. J. G.; Moura, I. Formate dehydrogenase from Desulfovibrio desulfuricans ATCC 27774: isolation and spectroscopic characterization of the active sites (heme, iron-sulfur centers and molybdenum). JBIC, J. Biol. Inorg. Chem. 1997, 2, 198-208.
(20) Maia, L. B.; Fonseca, L.; Moura, I.; Moura, J. J. G. Reduction of Carbon Dioxide by a Molybdenum-Containing Formate Dehydrogenase: A Kinetic and Mechanistic Study. J. Am. Chem. Soc. 2016, 138, 8834-8846.
(21) Riederer-Henderson, M. A.; Peck Jr, H. D. In Vitro Requirements for Formate Dehydrogenase Activity from Desulfovibrio. Can. J. Microbiol. 1986, 32, 425-429.
(22) Almendra, M. J.; Brondino, C. D.; Gavel, O.; Pereira, A. S.; Tavares, P.; Bursakov, S.; Duarte, R.; Caldeira, J.; Moura, J. J. G.; Moura, I. Purification and characterization of a tungsten-containing formate dehydrogenase from Desulfovibrio gigas. Biochemistry 1999, 38, 16366-16372.
(23) Oliveira, A. R.; Mota, C.; Mourato, C.; Domingos, R. M.; Santos, M. F. A.; Gesto, D.; Guigliarelli, B.; Santos-Silva, T.; Romão, M. J.; Cardoso Pereira, I. A. Toward the Mechanistic Understanding of Enzymatic CO₂ Reduction. ACS Catal. 2020, 10, 3844-3856. (24) Rivas, M. G.; González, P. J.; Brondino, C. D.; Moura, J. J. G.; Moura, I. EPR characterization of the molybdenum(V) forms of formate dehydrogenase from Desulfovibrio desulfuricans ATCC 27774 upon formate reduction. J. Inorg. Biochem. 2007, 101, 1617-1622.
(25) de Bok, F. A. M.; Hagedoorn, P.-L.; Silva, P. J.; Hagen, W. R.; Schiltz, E.; Fritsche, K.; Stams, A. J. M. Two W-containing formate dehydrogenases (CO₂-reductases) involved in syntrophic propionate oxidation by Syntrophobacter fumaroxidans. Eur. J. Biochem. 2003, 270, 2476-2485.
(26) Yu, X.; Niks, D.; Mulchandani, A.; Hille, R. Efficient reduction of CO₂ by the molybdenum-containing formate dehydrogenase from Cupriavidus necator (Ralstonia eutropha). J. Biol. Chem. 2017, 292, 16872-16879.
(27) Bassegoda, A.; Madden, C.; Wakerley, D. W.; Reisner, E.; Hirst, J. Reversible interconversion of CO₂ and formate by a molybdenum-containing formate dehydrogenase. J. Am. Chem. Soc. 2014, 136, 15473-15476.
(28) Axley, M. J.; Grahame, D. A. Kinetics for formate dehydrogenase of Escherichia coli formate-hydrogenlyase. J. Biol. Chem. 1991, 266, 13731-13736.
(29) Kpebe, A.; Benvenuti, M.; Guendon, C.; Rebai, A.; Fernandez, V.; Le Laz, S.; Etienne, E.; Guigliarelli, B.; Garcia-Molina, G.; de Lacey, A. L.; Baffert, C.; Brugna, M. A new mechanistic model for an O₂-protected electron-bifurcating hydrogenase, Hnd from Desulfovibrio fructosovorans. Biochim. Biophys. Acta, Bioenerg. 2018, 1859, 1302-1312.
(30) Axley, M. J.; Grahame, D. A.; Stadtman, T. C. Escherichia coli formate-hydrogen lyase. Purification and properties of the selenium-dependent formate dehydrogenase component. J. Biol. Chem. 1990, 265, 18213-18218.
(31) Khangulov, S. V.; Gladyshev, V. N.; Dismukes, G. C.; Stadtman, T. C. Selenium-containing formate dehydrogenase H from Escherichia coli: a molybdopterin enzyme that catalyzes formate oxidation without oxygen transfer. Biochemistry 1998, 37, 3518-3528.
(32) Friedebold, J.; Bowien, B. Physiological and biochemical characterization of the soluble formate dehydrogenase, a molybdoenzyme from Alcaligenes eutrophus. J. Bacteriol. 1993, 175, 4719-4728.
(33) Scherer, P. A.; Thauer, R. K. Purification and properties of reduced ferredoxin: CO₂ oxidoreductase from Clostridium pasteurianum, a molybdenum iron-sulfur-protein. Eur. J. Biochem. 1978, 85, 125-135.
(34) Kroger, A.; Winkler, E.; Innerhofer, A.; Hackenberg, H.; Schagger, H. The formate dehydrogenase involved in electron transport from formate to fumarate in Vibrio succinogenes. Eur. J. Biochem. 1979, 94, 465-475.
(35) Jollie, D. R.; Lipscomb, J. D. Formate dehydrogenase from Methylosinus trichosporium OB3b. Methods Enzymol. 1990, 188, 331-334.
(36) Jollie, D. R.; Lipscomb, J. D. Formate dehydrogenase from Methylosinus trichosporium OB3b. Purification and spectroscopic characterization of the cofactors. J. Biol. Chem. 1991, 266, 21853-21863.
(37) Yoch, D. C.; Chen, Y. P.; Hardin, M. G. Formate dehydrogenase from the methane oxidizer Methylosinus trichosporium OB3b. J. Bacteriol. 1990, 172, 4456-4463.
(38) Blanchard, J. S.; Cleland, W. W. Kinetic and chemical mechanisms of yeast formate dehydrogenase. Biochemistry 1980, 19, 3543-3550.
(39) Robinson, W. E.; Bassegoda, A.; Blaza, J. N.; Reisner, E.; Hirst, J. Understanding How the Rate of C—H Bond Cleavage Affects Formate Oxidation Catalysis by a Mo-Dependent Formate Dehydrogenase. J. Am. Chem. Soc. 2020, 142, 12226-12236.
(40) Hartmann, T.; Leimkühler, S. The oxygen-tolerant and NAD+-dependent formate dehydrogenase from Rhodobacter capsulatus is able to catalyze the reduction of CO² to formate. FEBS J. 2013, 280, 6083-6096.
(41) Stickland, L. H. The bacterial decomposition of formic acid. Biochem. J. 1929, 23, 1187-1198.
(42) Stephenson, M.; Stickland, L. H. Hydrogenlyases: Bacterial enzymes liberating molecular hydrogen. Biochem. J. 1932, 26, 712-724.
(43) Gale, E. F. Formic dehydrogenase of Bacterium coli: its inactivation by oxygen and its protection in the bacterial cell. Biochem. J. 1939, 33, 1012-1027.
(44) Pinsent, J. The need for selenite and molybdate in the formation of formic dehydrogenase by members of the coli-aerogenes group of bacteria. Biochem. J. 1954, 57, 10-16.
(45) Itagaki, E.; Fujita, T.; Sato, R. Solubilization and properties of formate dehydrogenase and cytochrome b1 from Escherichia coli. J. Biochem. 1962, 52, 131-141.
(46) Linnane, A. W.; Wrigley, C. W. Fragmentation of the Electron Transport Chain of Escherichia Coli. Preparation of a Soluble Formate Dehydrogenase-Cytochrome B1 Complex. Biochim. Biophys. Acta 1963, 77, 408-418.
(47) Gray, C. T.; Gest, H. Biological Formation of Molecular Hydrogen. Science 1965, 148, 186-192.
(48) Ruiz-Herrera, J.; Alvarez, A. A physiological study of formate dehydrogenase, formate oxidase, and hydrogenlyase from Escherichia coli K-12. Antonie van Leeuwenhoek 1972, 38, 479-491.
(49) Ljungdahl, L. G. Formate Dehydrogenases: Role of Molybdenum, Tungsten, and Selenium. In Molybdenum and Molybdenum-Containing Enzymes; Coughlan, M. P., Ed.; Pergamon: New York, 1980; pp 463-486.
(50) Pichinoty, F. Recherche des activites formiate-oxydase, hydrogene-lyase, hydrogenase et formiate-deshydrogenase chez quelques enterobacteriaceae. Ann. Inst. Pasteur 1969, 117, 3-15.
(51) Stewart, V. Nitrate respiration in relation to facultative metabolism in enterobacteria. Microbiol. Rev. 1988, 52, 190-232.
(52) Finney, A. J.; Sargent, F. Formate hydrogenlyase: A group 4[NiFe]-hydrogenase in tandem with a formate dehydrogenase. Adv. Microb. Physiol. 2019, 74, 465-486.
(53) Sawers, G.; Heider, J.; Zehelein, E.; Bock, A. Expression and operon structure of the sel genes of Escherichia coli and identification of a third selenium-containing formate dehydrogenase isoenzyme. J. Bacteriol. 1991, 173, 4983-4993.
(54) Soboh, B.; Pinske, C.; Kuhns, M.; Waclawek, M.; Ihling, C.; Trchounian, K.; Trchounian, A.; Sinz, A.; Sawers, G. The respiratory molybdo-selenoprotein formate dehydrogenases of Escherichia coli have hydrogen: benzyl viologen oxidoreductase activity. BMC Microbiol. 2011, 11, 173.
(55) Abaibou, H.; Pommier, J.; Benoit, S.; Giordano, G.; Mandrand-Berthelot, M. A. Expression and characterization of the Escherichia coli fdo locus and a possible physiological role for aerobic formate dehydrogenase. J. Bacteriol. 1995, 177, 7141-7149.
(56) Benoit, S.; Abaibou, H.; Mandrand-Berthelot, M.-A. Topological analysis of the aerobic membrane-bound formate dehydrogenase of Escherichia coli. J. Bacteriol. 1998, 180, 6625-6634.
(57) Unden, G.; Steinmetz, P. A.; Degreif-Dunnwald, P. The Aerobic and Anaerobic Respiratory Chain of Escherichia coli and Salmonella enterica: Enzymes and Energetics. EcoSal Plus 2014, 6. DOI: 10.1128/ecosalplus.ESP-0005-2013
(58) Richardson, D.; Sawers, G. Structural biology. PMF through the redox loop. Science 2002, 295, 1842-1843.
(59) Yagi, T. Formate: cytochrome oxidoreductase of Desulfovibrio vulgaris. J. Biochem. 1969, 66, 473-478.
(60) Yagi, T. Monoheme cytochromes. Methods Enzymol. 1994, 243, 104-118.
(61) Keller, K. L.; Wall, J. D.; Chhabra, S. Methods for engineering sulfate reducing bacteria of the genus Desulfovibrio. Methods Enzymol. 2011, 497, 503-517.
(62) Rabus, R.; Venceslau, S. S.; Wohlbrand, L.; Voordouw, G.; Wall, J. D.; Pereira, I. A. C. A Post-Genomic View of the Ecophysiology, Catabolism and Biotechnological Relevance of Sulphate-Reducing Prokaryotes. Adv. Microb. Physiol. 2015, 66, 55-321.
(63) Price, M. N.; Wetmore, K. M.; Waters, R. J.; Callaghan, M.; Ray, J.; Liu, H.; Kuehl, J. V.; Melnyk, R. A.; Lamson, J. S.; Suh, Y.; Carlson, H. K.; Esquivel, Z.; Sadeeshkumar, H.; Chakraborty, R.; Zane, G. M.; Rubin, B. E.; Wall, J. D.; Visel, A.; Bristow, J.; Blow, M. J.; Arkin, A. P.; Deutschbauer, A. M. Mutant phenotypes for thousands of bacterial genes of unknown function. Nature 2018, 557, 503-509.
(64) Lefevre, C. T.; Howse, P. A.; Schmidt, M. L.; Sabaty, M.; Menguy, N.; Luther, G. W., III; Bazylinski, D. A. Growth of magnetotactic sulfate-reducing bacteria in oxygen concentration gradient medium. Environ. Microbiol. Rep. 2016, 8, 1003-1015.
(65) Schoeffler, M.; Gaudin, A.-L.; Ramel, F.; Valette, O.; Denis, Y.; Hania, W. B.; Hirschler-Réa, A.; Dolla, A. Growth of an anaerobic sulfate-reducing bacterium sustained by oxygen respiratory energy conservation after O₂-driven experimental evolution. Environ. Microbiol. 2019, 21, 360-373.
(66) Muyzer, G.; Stams, A. J. M. The ecology and biotechnology of sulphate-reducing bacteria. Nat. Rev. Microbiol. 2008, 6, 441-454.
(67) Verhagen, M. F. J. M.; Wolbert, R. B. G.; Hagen, W. R. Cytochrome c553 from Desulfovibrio vulgaris (Hildenborough). Electrochemical properties and electron transfer with hydrogenase. Eur. J. Biochem. 1994, 221, 821-829.
(68) da Silva, S. M.; Pimentel, C.; Valente, F. M. A.; Rodrigues-Pousada, C.; Pereira, I. A. C. Tungsten and molybdenum regulation of formate dehydrogenase expression in Desulfovibrio vulgaris Hildenborough. J. Bacteriol. 2011, 193, 2909-2916.
(69) Heidelberg, J. F.; Seshadri, R.; Haveman, S. A.; Hemme, C. L.; Paulsen, I. T.; Kolonay, J. F.; Eisen, J. A.; Ward, N.; Methe, B.; Brinkac, L. M.; Daugherty, S. C.; Deboy, R. T.; Dodson, R. J.; Durkin, A. S.; Madupu, R.; Nelson, W. C.; Sullivan, S. A.; Fouts, D.; Haft, D. H.; Selengut, J.; Peterson, J. D.; Davidsen, T. M.; Zafar, N.; Zhou, L.; Radune, D.; Dimitrov, G.; Hance, M.; Tran, K.; Khouri, H.; Gill, J.; Utterback, T. R.; Feldblyum, T. V.; Wall, J. D.; Voordouw, G.; Fraser, C. M. The genome sequence of the anaerobic, sulfate-reducing bacterium Desulfovibrio vulgaris Hildenborough. Nat. Biotechnol. 2004, 22, 554-559.
(70) López, S.; Prieto, M.; Dijkstra, J.; Dhanoa, M. S.; France, J. Statistical evaluation of mathematical models for microbial growth. Int. J. Food Microbiol. 2004, 96, 289-300.
(71) de Bok, F. A. M.; Roze, E. H. A.; Stams, A. J. M. Hydrogenases and formate dehydrogenases of Syntrophobacter fumaroxidans. Antonie van Leeuwenhoek 2002, 81, 283-291.
(72) Hartwig, S.; Pinske, C.; Sawers, R. G. Chromogenic assessment of the three molybdo-selenoprotein formate dehydrogenases in Escherichia coli. Biochem. Biophys. Rep. 2015, 1, 62-67.
(73) Enoch, H. G.; Lester, R. L. The purification and properties of formate dehydrogenase and nitrate reductase from Escherichia coli. J. Biol. Chem. 1975, 250, 6693-6705.
(74) Pinske, C.; Jaroschinsky, M.; Sargent, F.; Sawers, G. Zymographic differentiation of [NiFe]-hydrogenases 1, 2 and 3 of Escherichia coli K-12. BMC Microbiol. 2012, 12, 134.
(75) Schafer, C.; Friedrich, B.; Lenz, 0. Novel, oxygen-insensitive group 5 [NiFe]-hydrogenase in Ralstonia eutropha. Appl. Environ. Microbiol. 2013, 79, 5137-5145.
(76) Altman, F. P. Tetrazolium Salts and Formazans; Gustav Fischer Verlag: Stuttgart, 1976.
(77) Hagen, W. R. Tungsten-Containing Enzymes. In Molybdenum and Tungsten Enzymes: Biochemistry; Hille, R., Schulzke, C., Kirk, M. L., Eds.; The Royal Society of Chemistry: Cambridge, 2017; pp 313-342.
(78) Gladyshev, V. N.; Boyington, J. C.; Khangulov, S. V.; Grahame, D. A.; Stadtman, T. C.; Sun, P. D. Characterization of crystalline formate dehydrogenase H from Escherichia coli. Stabilization, EPR spectroscopy, and preliminary crystallographic analysis. J. Biol. Chem. 1996, 271, 8095-8100. (79) Laukel, M.; Chistoserdova, L.; Lidstrom, M. E.; Vorholt, J. A. The tungsten-containing formate dehydrogenase from Methylobacterium extorquens AMI: purification and properties. Eur. J. Biochem. 2003, 270, 325-333.
(80) Orme-Johnson, W. H. Iron-sulfur proteins: structure and function. Annu. Rev. Biochem. 1973, 42, 159-204.
(81) Sweeney, W. V.; Rabinowitz, J. C. Proteins containing 4Fe-4S clusters: an overview. Annu. Rev. Biochem. 1980, 49, 139-161.
(82) Raaijmakers, H.; Teixeira, S.; Dias, J. M.; Almendra, M. J.; Brondino, C. D.; Moura, I.; Moura, J. J. G.; Romão, M. J. Tungstencontaining formate dehydrogenase from Desulfovibrio gigas: metal identification and preliminary structural data by multi-wavelength crystallography. J. Biol. Inorg Chem. 2001, 6, 398-404.
(83) Koehler, B. P.; Mukund, S.; Conover, R. C.; Dhawan, I. K.; Roy, R.; Adams, M. W. W.; Johnson, M. K. Spectroscopic characterization of the tungsten and iron centers in aldehyde ferredoxin oxidoreductases from two hyperthermophilic archaea. J. Am. Chem. Soc. 1996, 118, 12391-12405.
(84) Johnson, K. A. New standards for collecting and fitting steady state kinetic data. Beilstein J. Org. Chem. 2019, 15, 16-29.
(85) Silveira, C. M.; Besson, S.; Moura, I.; Moura, J. J.; Almeida, M. G. Measuring the cytochrome C nitrite reductase activity-practical considerations on the enzyme assays. Bioinorg. Chem. Appl. 2010, 1-8.
(86) Prince, R. C.; Linkletter, S. J. G.; Dutton, P. L. The thermodynamic properties of some commonly used oxidation-reduction mediators, inhibitors and dyes, as determined by polarography. Biochim. Biophys. Acta 1981, 635, 132-148.
(87) Duggleby, R. G.; Clarke, R. B. Experimental designs for estimating the parameters of the Michaelis-Menten equation from progress curves of enzyme-catalyzed reactions. Biochim. Biophys. Acta 1991, 1080, 231-236.
(88) Schweins, T.; Geyer, M.; Scheffzek, K.; Warshel, A.; Kalbitzer, H. R.; Wittinghofer, A. Substrate-assisted catalysis as a mechanism for GTP hydrolysis of p21ras and other GTP-binding proteins. Nat. Struct. Biol. 1995, 2, 36-44.
(89) Johnson, K. A. Fitting enzyme kinetic data with KinTek Global Kinetic Explorer. Methods Enzymol. 2009, 467, 601-626.
(90) Stroberg, W.; Schnell, S. On the estimation errors of KM and V from time-course experiments using the Michaelis-Menten equation. Biophys. Chem. 2016, 219, 17-27.
(91) Jahn, B.; Jonasson, N. S. W.; Hu, H.; Singer, H.; Pol, A.; Good, N. M.; den Camp, H. J. M. O.; Martinez-Gomez, N. C.; Daumann, L. J. Understanding the chemistry of the artificial electron acceptors PES, PMS, DCPIP and Wurster's Blue in methanol dehydrogenase assays. J. Biol. Inorg Chem. 2020, 25, 199-212.
(92) Peck, H. D., Jr.; Gest, H. Formic dehydrogenase and the hydrogenlyase enzyme complex in coli-aerogenes bacteria. J. Bacteriol. 1957, 73, 706-721.
(93) Xiang, D.; Magana, D.; Dyer, R. B. CO2 reduction catalyzed by mercaptopteridine on glassy carbon. J. Am. Chem. Soc. 2014, 136, 14007-14010.
(94) Merritt, M. E.; Harrison, C.; Storey, C.; Jeffrey, F. M.; Sherry, A. D.; Malloy, C. R. Hyperpolarized 13C allows a direct measure of flux through a single enzyme-catalyzed step by NMR. Proc. Natl. Acad. Sci. U.S.A. 2007, 104, 19773-19777.
(95) Niekus, H. G. D.; Stouthamer, A. H. Formate oxidase in glutaraldehyde-treated Campylobacter sputorum subspecies bubulus. FEMS Microbiol. Lett. 1981, 11, 83-87.
(96) Goodhew, C. F.; elKurdi, A. B.; Pettigrew, G. W. The microaerophilic respiration of Campylobacter mucosalis. Biochim. Biophys. Acta 1988, 933, 114-123.
(97) Weissgerber, T. L.; Winham, S. J.; Heinzen, E. P.; Milin-Lazovic, J. S.; Garcia-Valencia, O.; Bukumiric, Z.; Savic, M. D.; Garovic, V. D.; Milic, N. M. Reveal, Don't Conceal: Transforming Data Visualization to Improve Transparency. Circulation 2019, 140, 1506-1518.
(98) Miner, K. D.; Mukherjee, A.; Gao, Y.-G.; Null, E. L.; Petrik, I. D.; Zhao, X.; Yeung, N.; Robinson, H.; Lu, Y. A designed functional metalloenzyme that reduces O2 to H2O with over one thousand turnovers. Angew. Chem., Int. Ed. Engl. 2012, 51, 5589-5592.
(99) Kakeshpour, T.; Bax, A. NMR characterization of H₂O₂ hydrogen exchange. J. Magn. Reson. 2021, 333, 107092.
(100) Kalyanaraman, B.; Darley-Usmar, V.; Davies, K. J. A.; Dennery, P. A.; Forman, H. J.; Grisham, M. B.; Mann, G. E.; Moore, K.; Roberts, L. J., II; Ischiropoulos, H. Measuring reactive oxygen and nitrogen species with fluorescent probes: challenges and limitations. Free Radical Biol. Med. 2012, 52, 1-6.
(101) Wulff, P.; Day, C. C.; Sargent, F.; Armstrong, F. A. How oxygen reacts with oxygen-tolerant respiratory [NiFe]-hydrogenases. Proc. Natl. Acad. Sci. U.S.A. 2014, 111, 6606-6611.
(102) Zielonka, J.; Sikora, A.; Joseph, J.; Kalyanaraman, B. Peroxynitrite is the major species formed from different flux ratios of co-generated nitric oxide and superoxide: direct reaction with boronate-based fluorescent probe. J. Biol. Chem. 2010, 285, 14210-14216.
(103) Tarpey, M. M.; Fridovich, I. Methods of detection of vascular reactive species: nitric oxide, superoxide, hydrogen peroxide, and peroxynitrite. Circ. Res. 2001, 89, 224-236.
(104) Azzi, A.; Montecucco, C.; Richter, C. The use of acetylated ferricytochrome c for the detection of superoxide radicals produced in biological membranes. Biochem. Biophys. Res. Commun. 1975, 65, 597-603.
(105) Lu, Z.; Imlay, J. A. When anaerobes encounter oxygen: mechanisms of oxygen toxicity, tolerance and defence. Nat. Rev. Microbiol. 2021, 19, 774-785.
(106) Koppenol, W. H.; Stanbury, D. M.; Bounds, P. L. Electrode potentials of partially reduced oxygen species, from dioxygen to water. Free Radical Biol. Med. 2010, 49, 317-322.
(107) McDonald, A. G.; Boyce, S.; Tipton, K. F. ExplorEnz: the primary source of the IUBMB enzyme list. Nucleic Acids Res. 2009, 37, D593-D597.
(108) Lu, Y.; Koo, J. O₂ sensitivity and H₂ production activity of hydrogenases-A review. Biotechnol. Bioeng. 2019, 116, 3124-3135.
(109) Shaw, R. W.; Rife, J. E.; O'Leary, M. H.; Beinert, H. Oxidation of reduced cytochrome c oxidase with ¹⁸O₂. A search for mu-oxo-bridged metal species in the oxidized enzyme. J. Biol. Chem. 1981, 256, 1105-1107.
(110) Lauterbach, L.; Lenz, O. Catalytic production of hydrogen peroxide and water by oxygen-tolerant [NiFe]-hydrogenase during H₂ cycling in the presence of O₂. J. Am. Chem. Soc. 2013, 135, 17897-17905.
(111) Massey, V.; Harris, C. M. Milk xanthine oxidoreductase: the first one hundred years. Biochem. Soc. Trans. 1997, 25, 750-755.
(112) Pimviriyakul, P.; Chaiyen, P. Overview of flavin-dependent enzymes. Enzymes 2020, 47, 1-36.
(113) Liu, J.; Chakraborty, S.; Hosseinzadeh, P.; Yu, Y.; Tian, S.; Petrik, I.; Bhagi, A.; Lu, Y. Metalloproteins containing cytochrome, iron-sulfur, or copper redox centers. Chem. Rev. 2014, 114, 4366-4469.
(114) Radi, R.; Thomson, L.; Rubbo, H.; Prodanov, E. Cytochrome c catalyzed oxidation of organic molecules by hydrogen peroxide. Arch. Biochem. Biophys. 1991, 288, 112-117.
(115) Velayutham, M.; Hemann, C.; Zweier, J. L. Removal of H(2)O(2) and generation of superoxide radical: role of cytochrome c and NADH. Free Radical Biol. Med. 2011, 51, 160-170.
(116) Raaijmakers, H.; Macieira, S.; Dias, J. M.; Teixeira, S.; Bursakov, S.; Huber, R.; Moura, J. J. G.; Moura, I.; Romáo, M. J. Gene sequence and the 1.8 A crystal structure of the tungsten-containing formate dehydrogenase from Desulfovibrio gigas. Structure 2002, 10, 1261-1272.
(117) Radon, C.; Mittelstädt, G.; Duffus, B. R.; Burger, J.; Hartmann, T.; Mielke, T.; Teutloff, C.; Leimkühler, S.; Wendler, P. Cryo-EM structures reveal intricate Fe—S cluster arrangement and charging in Rhodobacter capsulatus formate dehydrogenase. Nat. Commun. 2020, 11, 1912.
(118) Lee, D.-S.; Nioche, P.; Hamberg, M.; Raman, C. S. Structural insights into the evolutionary paths of oxylipin biosynthetic enzymes. Nature 2008, 455, 363-368.
(119) Sommerhalter, M.; Lieberman, R. L.; Rosenzweig, A. C. X-ray crystallography and biological metal centers: is seeing believing? Inorg. Chem. 2005, 44, 770-778.
(120) Bowman, S. E. J.; Bridwell-Rabb, J.; Drennan, C. L. Metalloprotein Crystallography: More than a Structure. Acc. Chem. Res. 2016, 49, 695-702.
(121) Yang, J. Y.; Kerr, T. A.; Wang, X. S.; Barlow, J. M. Reducing CO₂ to HCO₂(−) at Mild Potentials: Lessons from Formate Dehydrogenase. J. Am. Chem. Soc. 2020, 142, 19438-19445.
(122) Palmer, G.; Olson, J. S. Concepts and approaches to the understanding of electron transfer processes in enzymes containing multiple redox centers. In Molybdenum and Molybdenum-Containing Enzymes; Coughlan, M. P., Ed.; Pergamon Press: Oxford, 1980; pp 187-220.
(123) Page, C. C.; Moser, C. C.; Chen, X.; Dutton, P. L. Natural engineering principles of electron tunnelling in biological oxidation-reduction. Nature 1999, 402, 47-52.
(124) Osyczka, A.; Moser, C. C.; Daldal, F.; Dutton, P. L. Reversible redox energy coupling in electron transfer chains. Nature 2004, 427, 607-612.
(125) Byrne, R. S.; Hänsch, R.; Mendel, R. R.; Hille, R. Oxidative half-reaction of Arabidopsis thaliana sulfite oxidase: generation of superoxide by a peroxisomal enzyme. J. Biol. Chem. 2009, 284, 35479-35484.
(126) Nicolet, Y.; Fontecilla-Camps, J. C. Iron-sulfur clusters and molecular oxygen:function, adaptation, degradation, and repair. In Iron-Sulfur Clusters in Chemistry and Biology; Rouault, T. A., Ed., 2014; pp 359-385.
(127) Kayastha, K.; Vitt, S.; Buckel, W.; Ermler, U. Flavins in the electron bifurcation process. Arch. Biochem. Biophys. 2021, 701, 108796.
(128) Crofts, A. R. The modified Q-cycle: A look back at its development and forward to a functional model. Biochim. Biophys. Acta, Bioenerg. 2021, 1862, 148417.
(129) Sarewicz, M.; Pintscher, S.; Pietras, R.; Borek, A.; Bujnowicz, Ł.; Hanke, G.; Cramer, W. A.; Finazzi, G.; Osyczka, A. Catalytic Reactions and Energy Conservation in the Cytochrome bc1 and b6f Complexes of Energy-Transducing Membranes. Chem. Rev. 2021, 121, 2020-2108.
(130) Buckel, W.; Thauer, R. K. Flavin-Based Electron Bifurcation, A New Mechanism of Biological Energy Coupling. Chem. Rev. 2018, 118, 3862-3886.
(131) Wise, C. E.; Ledinina, A. E.; Yuly, J. L.; Artz, J. H.; Lubner, C. E. The role of thermodynamic features on the functional activity of electron bifurcating enzymes. Biochim. Biophys. Acta, Bioenerg. 2021, 1862, 148377.
(132) Baymann, F.; Schoepp-Cothenet, B.; Duval, S.; Guiral, M.; Brugna, M.; Baffert, C.; Russell, M. J.; Nitschke, W. On the Natural History of Flavin-Based Electron Bifurcation. Front Microbiol. 2018, 9, 1357.
(133) Ian Ragan, C. Structure of NADH-ubiquinone reductase (complex I). Curr. Top. Bioenerg. 1987, 15, 1-36.
(134) Nitschke, W.; Russell, M. J. Redox bifurcations: mechanisms and importance to life now, and at its origin: a widespread means of energy conversion in biology unfolds. Bioessays 2012, 34, 106-109.
(135) Peters, J. W.; Beratan, D. N.; Schut, G. J.; Adams, M. W. W. On the nature of organic and inorganic centers that bifurcate electrons, coupling exergonic and endergonic oxidation-reduction reactions. Chem. Commun. 2018, 54, 4091-4099.
(136) Yuly, J. L.; Zhang, P.; Ru, X.; Terai, K.; Singh, N.; Beratan, D. N. Efficient and reversible electron bifurcation with either normal or inverted potentials at the bifurcating cofactor. Chem 2021, 7, 1870.
(137) Agarwal, R. G.; Coste, S. C.; Groff, B. D.; Heuer, A. M.; Noh, H.; Parada, G. A.; Wise, C. F.; Nichols, E. M.; Warren, J. J.; Mayer, J. M. Free Energies of Proton-Coupled Electron Transfer Reagents and Their Applications. Chem. Rev. 2022, 122, 1-49.
(138) Bol, E.; Bevers, L. E.; Hagedoorn, P.-L.; Hagen, W. R. Redox chemistry of tungsten and iron-sulfur prosthetic groups in Pyrococcus furiosus formaldehyde ferredoxin oxidoreductase. J. Biol. Inorg Chem. 2006, 11, 999-1006.
(139) Li, F.; Hinderberger, J.; Seedorf, H.; Zhang, J.; Buckel, W.; Thauer, R. K. Coupled ferredoxin and crotonyl coenzyme A (CoA) reduction with NADH catalyzed by the butyryl-CoA dehydrogenase/Etf complex from Clostridium kluyveri. J. Bacteriol. 2008, 190, 843-850.
(140) Aboulnaga, E.-H.; Pinkenburg, O.; Schiffels, J.; El-Refai, A.; Buckel, W.; Selmer, T. Effect of an oxygen-tolerant bifurcating butyryl coenzyme A dehydrogenase/electron-transferring flavoprotein complex from Clostridium difficile on butyrate production in Escherichia coli. J. Bacteriol. 2013, 195, 3704-3713.
(141) Chowdhury, N. P.; Kahnt, J.; Buckel, W. Reduction of ferredoxin or oxygen by flavin-based electron bifurcation in Megasphaera elsdenii. FEBS J. 2015, 282, 3149-3160.
(142) Yuly, J. L.; Zhang, P.; Lubner, C. E.; Peters, J. W.; Beratan, D. N. Universal free-energy landscape produces efficient and reversible electron bifurcation. Proc. Natl. Acad. Sci. U.S.A. 2020, 117, 21045-21051.
(143) Rutherford, A. W.; Osyczka, A.; Rappaport, F. Back-reactions, short-circuits, leaks and other energy wasteful reactions in biological electron transfer: redox tuning to survive life in O(2). FEBS Lett. 2012, 586, 603-616.
(144) Gran-Scheuch, A.; Parra, L.; Fraaije, M. W. Systematic assessment of uncoupling in flavoprotein oxidases and monooxygenases. ACS Sustainable Chem. Eng. 2021, DOI: 10.1021/acssuschemeng. 1c02012.
(145) Pintscher, S.; Kuleta, P.; Cieluch, E.; Borek, A.; Sarewicz, M.; Osyczka, A. Tuning of Hemes b Equilibrium Redox Potential Is Not Required for Cross-Membrane Electron Transfer. J. Biol. Chem. 2016, 291, 6872-6881.
(146) Kint, N.; Morvan, C.; Martin-Verstraete, I. Oxygen response and tolerance mechanisms in Clostridioides difficile. Curr. Opin. Microbiol. 2022, 65, 175-182.
(147) Ledbetter, R. N.; Garcia Costas, A. M.; Lubner, C. E.; Mulder, D. W.; Tokmina-Lukaszewska, M.; Artz, J. H.; Patterson, A.; Magnuson, T. S.; Jay, Z. J.; Duan, H. D.; Miller, J.; Plunkett, M. H.; Hoben, J. P.; Barney, B. M.; Carlson, R. P.; Miller, A.-F.; Bothner, B.; King, P. W.; Peters, J. W.; Seefeldt, L. C. The Electron Bifurcating FixABCX Protein Complex from Azotobacter vinelandii: Generation of Low-Potential Reducing Equivalents for Nitrogenase Catalysis. Biochemistry 2017, 56, 4177-4190.
(148) Wang, Y.; Chen, X.; Spengler, K.; Terberger, K.; Boehm, M.; Appel, J.; Barske, T.; Timm, S.; Battchikova, N.; Hagemann, M.; Gutekunst, K. Pyruvate:ferredoxin oxidoreductase and low abundant ferredoxins support aerobic photomixotrophic growth in cyanobacteria. Elife 2022, 11, No. e71339.
(149) Zuchan, K.; Baymann, F.; Baffert, C.; Brugna, M.; Nitschke, W. The dyad of the Y-junction- and a flavin module unites diverse redox enzymes. Biochim. Biophys. Acta, Bioenerg. 2021, 1862, 148401.
(150) Chen, Y.-P.; Yoch, D. C. Reconstitution of electron transport system that couples formate oxidation to nitrogenase in Methylosinus trichosporium OB3b. J. Gen. Microbiol. 1988, 134, 3123-3128.
(151) Jollie, D. R. Structure and mechanism of formate dehydrogenase isolated from the methanotroph, Methylosinus trichosporium OB3b. PhD Dissertation, University of Minnesota, Minneapolis, 1992.
(152) Wang, S.; Huang, H.; Kahnt, J.; Thauer, R. K. Clostridium acidurici electron-bifurcating formate dehydrogenase. Appl. Environ. Microbiol. 2013, 79, 6176-6179.
(153) Jumper, J.; Evans, R.; Pritzel, A.; Green, T.; Figurnov, M.; Ronneberger, O.; Tunyasuvunakool, K.; Bates, R.; Zidek, A.; Potapenko, A.; Bridgland, A.; Meyer, C.; Kohl, S. A. A.; Ballard, A. J.; Cowie, A.; Romera-Paredes, B.; Nikolov, S.; Jain, R.; Adler, J.; Back, T.; Petersen, S.; Reiman, D.; Clancy, E.; Zielinski, M.; Steinegger, M.; Pacholska, M.; Berghammer, T.; Bodenstein, S.; Silver, D.; Vinyals, O.; Senior, A. W.; Kavukcuoglu, K.; Kohli, P.; Hassabis, D. Highly accurate protein structure prediction with AlphaFold. Nature 2021, 596, 583-589.
(154) Holm, L. Using Dali for Protein Structure Comparison. Methods Mol. Biol. 2020, 2112, 29-42.
(155) Reich, H. J.; Hondal, R. J. Why Nature Chose Selenium. ACS Chem. Biol. 2016, 11, 821-841.
(156) Evans, R. M.; Krahn, N.; Murphy, B. J.; Lee, H.; Armstrong, F. A.; Soll, D. Selective cysteine-to-selenocysteine changes in a [NiFe]-hydrogenase confirm a special position for catalysis and oxygen tolerance. Proc. Natl. Acad. Sci. U.S.A. 2021, 118, No. e2100921118.
(157) Sen, A.; Imlay, J. A. How Microbes Defend Themselves From Incoming Hydrogen Peroxide. Front Immunol. 2021, 12, 667343.
(158) Niekus, H. G. D.; Van Doorn, E.; De Vries, W.; Stouthamer, A. H. Aerobic growth of Campylobacter sputorum subspecies bubulus with formate. J. Gen. Microbiol. 1980, 118, 419-428.
(159) Ohta, H.; Gottschal, J. C. Formate oxidation by Wolinella recta ATCC 33238 with oxygen as electron acceptor. FEMS Microbiol. Lett. 1988, 50, 163-168.
(160) Hoffman, P. S.; Goodman, T. G. Respiratory physiology and energy conservation efficiency of Campylobacter jejuni. J. Bacteriol. 1982, 150, 319-326.
(161) Taylor, A. J.; Kelly, D. J. The function, biogenesis and regulation of the electron transport chains in Campylobacter jejuni: New insights into the bioenergetics of a major food-borne pathogen. Adv. Microb. Physiol. 2019, 74, 239-329.
(162) Khademian, M.; Imlay, J. A. Escherichia coli cytochrome c peroxidase is a respiratory oxidase that enables the use of hydrogen peroxide as a terminal electron acceptor. Proc. Natl. Acad. Sci. U.S.A. 2017, 114, E6922-E6931.
(163) del Barrio, M.; Guendon, C.; Kpebe, A.; Baffert, C.; Fourmond, V.; Brugna, M.; Léger, C. Valine to cysteine mutation further increases oxygen tolerance of Escherichia coli NiFe hydrogenase Hyd-1. ACS Catal. 2019, 9, 4084-4088.
(164) Nishikawa, K.; Ogata, H.; Higuchi, Y. Structural basis of the function of [NiFe]-hydrogenases. Chem. Lett. 2020, 49, 164-173.
(165) Noor, N. D. M.; Matsuura, H.; Nishikawa, K.; Tai, H.; Hirota, S.; Kim, J.; Kang, J.; Tateno, M.; Yoon, K.-S.; Ogo, S.; Kubota, S.; Shomura, Y.; Higuchi, Y. Redox-dependent conformational changes of a proximal [4Fe-4S] cluster in Hyb-type [NiFe]-hydrogenase to protect the active site from O2. Chem. Commun. 2018, 54, 12385-12388.
(166) Sakai, K.; Kitazumi, Y.; Shirai, O.; Takagi, K.; Kano, K. High-Power Formate/Dioxygen Biofuel Cell Based on Mediated Electron Transfer Type Bioelectrocatalysis. ACS Catal. 2017, 7, 5668-5673.
(167) Meneghello, M.; Léger, C.; Fourmond, V. Electrochemical Studies of CO₂-Reducing Metalloenzymes. Chemistry 2021, 27, 17542-17553.
(168) Ruth, J. C.; Spormann, A. M. Enzyme electrochemistry for industrial applications—A perspective on future area of focus. ACS Catal. 2021, 11, 5951-5967.
(169) Cracknell, J. A.; Vincent, K. A.; Ludwig, M.; Lenz, O.; Friedrich, B.; Armstrong, F. A. Enzymatic oxidation of H2 in atmospheric O₂: the electrochemistry of energy generation from trace H2 by aerobic microorganisms. J. Am. Chem. Soc. 2008, 130, 424-425.
(170) Vincent, K. A.; Cracknell, J. A.; Lenz, O.; Zebger, I.; Friedrich, B.; Armstrong, F. A. Electrocatalytic hydrogen oxidation by an enzyme at high carbon monoxide or oxygen levels. Proc. Natl. Acad. Sci. U.S.A. 2005, 102, 16951-16954.
(171) Vincent, K. A.; Cracknell, J. A.; Clark, J. R.; Ludwig, M.; Lenz, O.; Friedrich, B.; Armstrong, F. A. Electricity from low-level H2 in still air—an ultimate test for an oxygen tolerant hydrogenase. Chem. Commun. 2006, 5033-5035.
(172) Utterback, J. K.; Ruzicka, J. L.; Keller, H. R.; Pellows, L. M.; Dukovic, G. Electron Transfer from Semiconductor Nanocrystals to Redox Enzymes. Annu. Rev. Phys. Chem. 2020, 71, 335-359.
(173) Greene, B. L.; Vansuch, G. E.; Chica, B. C.; Adams, M. W. W.; Dyer, R. B. Applications of Photogating and Time Resolved Spectroscopy to Mechanistic Studies of Hydrogenases. Acc. Chem. Res. 2017, 50, 2718-2726.
(174) Falk, M.; Miller, A. G. Infrared spectrum of carbon dioxide in aqueous solution. Vib. Spectrosc. 1992, 4, 105-108.
(175) Riepe, M. E.; Wang, J. H. Infrared studies on the mechanism of action of carbonic anhydrase. J. Biol. Chem. 1968, 243, 2779-2787.
(176) Muthusamy, M.; Burrell, M. R.; Thorneley, R. N. F.; Bornemann, S. Real-time monitoring of the oxalate decarboxylase reaction and probing hydron exchange in the product, formate, using fourier transform infrared spectroscopy. Biochemistry 2006, 45, 10667-10673.
(177) Mulder, D. W.; Peters, J. W.; Raugei, S. Catalytic bias in oxidation-reduction catalysis. Chem. Commun. 2021, 57, 713-720.
(178) Fourmond, V.; Plumeré, N.; Léger, C. Reversible catalysis. Nat. Rev. Chem. 2021, 5, 348-360.
(179) Shafaat, H. S.; Yang, J. Y. Uniting biological and chemical strategies for selective CO₂ reduction. Nat. Catal. 2021, 4, 928-933.
(180) Magalon, A.; Alberge, F. Distribution and dynamics of OXPHOS complexes in the bacterial cytoplasmic membrane. Biochim. Biophys. Acta 2016, 1857, 198-213.
(181) Orme-Johnson, W. H.; Orme-Johnson, N. R. In Iron-Sulfur Proteins; Spiro, T. G., Ed.; Wiley-Interscience: New York, 1982; pp 67-96.
(182) Rutherfurd, S. M.; Dunn, B. M. Quantitative amino acid analysis. Curr. Protoc. Protein Sci. 2011, 63, 321.
(183) Beinert, H. Micro methods for the quantitative determination of iron and copper in biological material. Methods Enzymol. 1978, 54, 435-445.
(184) Beinert, H. Semi-micro methods for analysis of labile sulfide and of labile sulfide plus sulfane sulfur in unusually stable iron-sulfur proteins. Anal. Biochem. 1983, 131, 373-378.
(185) Jones, R. W.; Garland, P. B. Sites and specificity of the reaction of bipyridylium compounds with anaerobic respiratory enzymes of Escherichia coli. Effects of permeability barriers imposed by the cytoplasmic membrane. Biochem. J. 1977, 164, 199-211.
(186) Ghosh, R.; Quayle, J. R. Phenazine ethosulfate as a preferred electron acceptor to phenazine methosulfate in dye-linked enzyme assays. Anal. Biochem. 1979, 99, 112-117.
(187) Jahn, B.; Pol, A.; Lumpe, H.; Barends, T. R. M.; Dietl, A.; Hogendoorn, C.; Op den Camp, H. J. M.; Daumann, L. J. Similar but Not the Same: First Kinetic and Structural Analyses of a Methanol Dehydrogenase Containing a Europium Ion in the Active Site. Chembiochem 2018, 19, 1147.
(188) Olp, M. D.; Kalous, K. S.; Smith, B. C. ICEKAT: an interactive online tool for calculating initial rates from continuous enzyme kinetic traces. BMC Bioinf 2020, 21, 186.
(189) Stoll, S.; Schweiger, A. EasySpin, a comprehensive software package for spectral simulation and analysis in EPR. J. Magn. Reson. 2006, 178, 42-55.
(190) Brautigan, D. L.; Ferguson-Miller, S.; Margoliash, E. Mitochondrial cytochrome c: preparation and activity of native and chemically modified cytochromes c. Methods Enzymol. 1978, 53, 128-164.
(191) Edwards, E.; Bren, K., Light-driven catalysis with engineered enzymes and biomimetic systems, Biotechnology and Applied Biochemistry, Vol. 67, Issue 4, July/August 2020, 463-483.
(192) Haque, S.; Yasir, M.; Cosnier, S., Recent advancements in the field of flexible/wearable enzyme fuel cells, Biosensors and Bioelectronics, 214 (2002), 114545.
(193) Hardt, S.; Stapf, S; Filmon, D.; Birrell, J.; Rudiger, O.; Fourmond, V.; Leger, C.; Plumere, N., Reversible H2 oxidation and evolution by hydrogenase embedded in a redox polymer film, Nature Catalysis, Vol. 4, March 2021, 251-258.
(194) Voller, J.; Air-Stable [FeFe] hydrogenases, Natura Catalysis, Vol. 1, August 2018, 564.
(195) Plumere, N.; Rudiger, O.; Alsheikh, A.; Williams, R.; Vivekananthan, J.; Poller, S.; Schuhmann, W.; Lubitz, W., A redox hydrogel protects hydrogenase from high-potential deactivation and oxygen damage, Nature Chemistry, Vol. 6, September 2014, 822-827.
(196) Graham, J.; Niks, D.; Zane G.; Gui, Q.; Hom, K.; Hille, R.; Wall, J., Raman, C., How a Formate Dehydrogenase Responds to Oxygen: Unexpected O₂ Insensitivity of an Enzyme Harboring Tungstopterin, Selenocysteine, and [4Fe-4S] Clusters, ACS Catal. 2022, 12, 10449-10471.
(197) Milton, R., FAD-Dependent Glucose Dehydrogenase Immobilization and Mediation Within a Naphthoquinone Redox Polymer, Shelley D. Minteer (ed.), Enzyme Stabilization and Immobilization: Methods and Protocols, Methods in Molecular Biology, vol. 1504, DOI 10.1007/978-1-4939-6499-4_15, Springer Science+Business Media New York 2017
(198) Sokol, K.; Robinson, W.; Oliveira, A.; Warnan, J.; Nowaczyk, M.; Ruff, A.; Pereira, I.; Reisner, E, Photoreduction of CO₂ with a Formate Dehydrogenase Driven by Photosystem II Using a Semi-artificial Z-Scheme Architecture. J. Am. Chem. Soc., 2018, 140, 48, 16418-16422.
(199) Hillesland, K. L.; Lim, S.; Flowers, J. J.; Turkarslan, S.; Pinel, N.; Zane, G. M.; Elliott, N.; Qin, Y.; Wu, L.; Baliga, N. S.; Zhou, J.; Wall, J. D.; Stahl, D. A., Erosion of functional independence early in the evolution of a microbial mutualism. Proc Natl Acad Sci USA 2014, 111, 14822-7.
(200) Keller, K. L.; Bender, K. S.; Wall, J. D., Development of a markerless genetic exchange system for Desulfovibrio vulgaris Hildenborough and its use in generating a strain with increased transformation efficiency. Appl Environ Microbiol 2009, 75, 7682-91.
(201) Li, M. Z.; Elledge, S. J., Harnessing homologous recombination in vitro to generate recombinant DNA via SLIC. Nat Methods 2007, 4, 251-6.
(202) Zane, G. M.; Yen, H. C.; Wall, J. D., Effect of the deletion of qmoABC and the promoter-distal gene encoding a hypothetical protein on sulfate reduction in Desulfovibrio vulgaris Hildenborough. Appl Environ Microbiol 2010, 76, 5500-9.
(203) Studier, F. W., Protein production by auto-induction in high density shaking cultures. Protein Expr Purif 2005, 41, 207-34.
(204) Parks, J. M.; Johs, A.; Podar, M.; Bridou, R.; Hurt, R. A., Jr.; Smith, S. D.; Tomanicek, S. J.; Qian, Y.; Brown, S. D.; Brandt, C. C.; Palumbo, A. V.; Smith, J. C.; Wall, J. D.; Elias, D. A.; Liang, L., The genetic basis for bacterial mercury methylation. Science 2013, 339, 1332-5.
(205) Teufel, F.; Almagro Armenteros, J. J.; Johansen, A. R.; Gislason, M. H.; Pihl, S. I.; Tsirigos, K. D.; Winther, O.; Brunak, S.; von Heijne, G.; Nielsen, H., SignalP 6.0 predicts all five types of signal peptides using protein language models. Nat Biotechnol 2022.
(206) Mota, C. S.; Rivas, M. G.; Brondino, C. D.; Moura, I.; Moura, J. J.; Gonzalez, P. J.; Cerqueira, N. M., The mechanism of formate oxidation by metal-dependent formate dehydrogenases. J Biol Inorg Chem 2011, 16, 1255-68.
(207) Mota, C. S.; Valette, O.; Gonzalez, P. J.; Brondino, C. D.; Moura, J. J.; Moura, I.; Dolla, A.; Rivas, M. G., Effects of molybdate and tungstate on expression levels and biochemical characteristics of formate dehydrogenases produced by Desulfovibrio alaskensis NCIMB 13491. J Bacteriol 2011, 193, 2917-23.
(208) Miller, M.; Robinson, W. E.; Oliveira, A. R.; Heidary, N.; Kornienko, N.; Warnan, J.; Pereira, I. A. C.; Reisner, E., Interfacing Formate Dehydrogenase with Metal Oxides for the Reversible Electrocatalysis and Solar-Driven Reduction of Carbon Dioxide. Angew Chem Int Ed Engl 2019, 58, 4601-4605.
(209) Szczesny, J.; Ruff, A.; Oliveira, A. R.; Pita, M.; Pereira, I. A.; De Lacey, A. L.; Schuhmann, W., Electroenzymatic CO2 fixation using redox polymer/enzyme-modified gas diffusion electrodes. ACS Energy Lett 2020, 5, 321-327.(210) Alvarez-Malmagro, J.; Oliveira, A. R.; Gutierrez-Sanchez, C.; Villajos, B.; Pereira, I. A. C.; Velez, M.; Pita, M.; De Lacey, A. L., Bioelectrocatalytic Activity of W-Formate Dehydrogenase Covalently Immobilized on Functionalized Gold and Graphite Electrodes. ACS Appl Mater Interfaces 2021, 13, 11891-11900.
(211) Rivas, M. G.; Gonzalez, P. J.; Brondino, C. D.; Moura, J. J.; Moura, I., EPR characterization of the molybdenum(V) forms of formate dehydrogenase from Desulfovibrio desulfuricans ATCC 27774 upon formate reduction. J Inorg Biochem 2007, 101, 1617-22.
(212) Lissolo, T.; Pulvin, S.; Thomas, D., Reactivation of the hydrogenase from Desulfovibrio gigas by hydrogen. Influence of redox potential. J Biol Chem 1984, 259, 11725-9.
(213) Tagawa, K.; Arnon, D. I., Oxidation-reduction potentials and stoichiometry of electron transfer in ferredoxins. Biochim Biophys Acta 1968, 153, 602-13.
(214) Jones, R. W., The topography of the membrane-bound hydrogenase of Escherichia coli explored by non-physiological electron acceptors [proceedings]. Biochem Soc Trans 1979, 7, 724-5.
(215) McKellar, R. C.; Sprott, G. D., Solubilization and properties of a particulate hydrogenase from Methanobacterium strain G2R. J Bacteriol 1979, 139, 231-8.
(216) Graf, E.-G.; Thauer, R. K., Hydrogenase from Methanobacterium thermoautotrophicum, a nickel-containing enzyme. FEBS Lett 1981, 136, 163-169.
(217) Arp, D. J.; Burris, R. H., Kinetic mechanism of the hydrogen-oxidizing hydrogenase from soybean nodule bacteroids. Biochemistry 1981, 20, 2234-40.
(218) Rosner, B. M.; Schink, B., Purification and characterization of acetylene hydratase of Pelobacter acetylenicus, a tungsten iron-sulfur protein. J Bacteriol 1995, 177, 5767-72.
(219) Mukund, S.; Adams, M. W., Glyceraldehyde-3-phosphate ferredoxin oxidoreductase, a novel tungsten-containing enzyme with a potential glycolytic role in the hyperthermophilic archaeon Pyrococcus furiosus. J Biol Chem 1995, 270, 8389-92.
(220) Gross, R.; Simon, J.; Kroger, A., Periplasmic methacrylate reductase activity in Wolinella succinogenes. Arch Microbiol 2001, 176, 310-3.
(221) Selwyn, M. J., A simple test for inactivation of an enzyme during assay. Biochim Biophys Acta 1965, 105, 193-5.
(222) Biofuel Cells: Materials and Challenges, Eds. Inamuddin, M. A. Ahamed, R. Boddula, M. Rezakazemi (2021), John Wiley & Sons.
(223) Li, W.; Sheng, G.; Liu, X.; Yu, H., Recent advances in the separators for microbial fuel cells, Bioresource Technology, Vol. 102, Issue 1, January 2011, p. 244-252.
(224) Milton, R. D.; Hickey, D. P.; Abdellaoui, S.; Lim, K.; Wu, F.; Tan, B.; Minteer, S. D., Rational design of quinones for high power density biofuel cells. Chemical science 2015, 6 (8), 4867-4875.
(225) Yuan, M.; Sahin, S.; Cai, R.; Abdellaoui, S.; Hickey, D. P.; Minteer, S. D.; Milton, R. D., Creating a Low-Potential Redox Polymer for Efficient Electroenzymatic CO2 Reduction. Angewandte Chemie 2018, 130 (22), 6692-6696.
(226) Quah, T.; Milton, R. D.; Abdellaoui, S.; Minteer, S. D., Bioelectrocatalytic NAD+/NADH interconversion: transformation of an enzymatic fuel cell into an enzymatic redox flow battery. Chemical Communications 2017, 53 (60), 8411-8414.

What is claimed is:

1. A biofuel cell, comprising:
a chamber,
a bioanode comprising formate dehydrogenase (DvH-FDH2) derived from Desulfovibrio vulgaris Hildenborough adsorbed thereon,
a biocathode comprising laccase, bilirubin oxidase, or cytochrome cbd oxidase (CydCBD) adsorbed thereon, and
an electrolyte comprising a buffer including formate,
wherein the bioanode and the biocathode are electrically connected to form an electric circuit, and
wherein the DvH-FDH2 is O₂-insensitive.

2. The biofuel cell of claim 1, wherein the DvH-FDH2 is an enzyme having a first subunit represented by SEQ ID NO: 31 and a second subunit represented by SEQ ID NO: 32.

3. The biofuel cell of claim 1, wherein the DvH-FDH2 has a first subunit having 90% or more identity to SEQ ID NO: 31 and a second subunit having 90% or more identity to SEQ ID NO: 32.

4. The biofuel cell of claim 1, wherein the biocathode comprises the CydCBD adsorbed thereon, the CydCBD being an enzyme having a first subunit represented by SEQ ID NO: 33 and a second subunit represented by SEQ ID NO: 34.

5. The biofuel cell of claim 1, wherein the CydCBD has a first subunit having 90% or more identity to SEQ ID NO: 33 and a second subunit having 90% or more identity to SEQ ID NO: 34.

6. The biofuel cell of claim 1, wherein at least one of the bioanode and the biocathode further comprises undecaheme cytochrome c (UHC) represented by SEQ ID NO: 35 adsorbed thereon.

7. The biofuel cell of claim 1, further comprising an O₂ bubbler or pump which provides Oz to the electrolyte.

8. The biofuel cell of claim 1, further comprising a gas permeable membrane disposed in the chamber between the bioanode and the biocathode.

9. An implantable device comprising the biofuel cell of claim 1.

10. The implantable medical device according to claim 9, wherein the implantable medical device is a contact lens.

11. The implantable medical device according to claim 9, wherein the implantable medical device is a pacemaker.

12. A method of generating electricity comprising:
providing the biofuel cell of claim 1, and
exposing the electrolyte to open air, or providing air to the electrolyte by an O₂ bubbler or pump.

13. A kit for generating hydrogen peroxide, comprising:
a matrix having formate dehydrogenase (DvH-FDH2) derived from Desulfovibrio vulgaris Hildenborough immobilized thereon, the DvH-FDH2 being O₂-insensitive, and
a source of oxygenated formate, and
an apparatus which flows oxygenated formate to the matrix.

14. A method of generating hydrogen peroxide, comprising:
providing the kit of claim 13,
flowing oxygenated formate to the matrix, and
collecting generated hydrogen peroxide.

15. A kit for detecting formate, comprising:
a reaction buffer,
a formate standard, and
formate dehydrogenase (DvH-FDH2) derived from Desulfovibrio vulgaris Hildenborough, the DvH-FDH2 being O₂-insensitive, and
a mediator dye.

16. A method of detecting formate in a sample, comprising:
providing the kit of claim 15,
obtaining a standard curve using the formate standard,
treating the sample with the reaction buffer, the DvH-FDH2, and the mediator,
providing air to the sample, and
detecting a change in color with a spectrophotometer to quantify an amount of the formate in the sample.

17. A device for converting carbon dioxide in air to formate, comprising:
a chamber,
an electrolyte comprising a buffer including formate,
a bioanode comprising an enzyme adsorbed thereon, the enzyme being capable of injecting electrons into the electrolyte,
a biocathode comprising formate dehydrogenase (DvH-FDH2) derived from Desulfovibrio vulgaris Hildenborough adsorbed thereon, and
an air supply which injects air containing carbon dioxide into the electrolyte,
wherein the bioanode and biocathode are electrically connected to an electric circuit, and
wherein the DvH-FDH2 is O₂-insensitive.

18. A method for converting carbon dioxide in air to formate, comprising:
providing the device of claim 17,
providing electrical power to the bioanode so that the bioanode generates electrons to transfer to the biocathode, and
injecting air containing carbon dioxide into the electrolyte.
